SEQUENCE LISTING (61 sequences)

SEQ ID NO: 1 - protein, 435 aa, Artificial Sequence
  Description of Artificial Sequence: Synthetic polypeptide
MEGGDQSEEEPRERSQAGGMGTLWSQESTPEERLPVEGSRPWAVARRVLTAILILGLLLCFSVLLFYNFQNCGPRPCETGGGGSGGGGSYPYDVPDYAGGGGSGGGGSADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 2 - protein, 443 aa, Artificial Sequence
  Description of Artificial Sequence: Synthetic polypeptide
MYGKIIFVLLLSEIVSISALLSEIVSISAQVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSSGGGGSGGGGSGGGGSDIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIKGGSGGSGGYPYDVPDYAGGSGGSGGMYGKIIFVLLLSEIVSISALSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 3 - protein, 348 aa, Erwinia chrysanthemi
MERWFKSLFVLVLFFVFTASAADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 4 - protein, 348 aa, Escherichia coli
MEFFKKTALAALVMGFSGAALALPNITILATGGTIAGGGDSATKSNYTVGKVGVENLVNAVPQLKDIANVKGEQVVNIGSQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 5 - protein, 346 aa, Erwinia carotovora
MFNALFVVVFVCFSSLANAAENLPNIVILATGGTIAGSAAANTQTTGYKAGALGVETLIQAVPELKTLANIKGEQVASIGSENMTSDVLLTLSKRVNELLARSDVDGVVITHGTDTLDESPYFLNLTVKSDKPVVFVAAMRPATAISADGPMNLYGAVKVAADKNSRGRGVLVVLNDRIGSARFISKTNASTLDTFKAPEEGYLGVIIGDKIYYQTRLDKVHTTRSVFDVTNVDKLPAVDIIYGYQDDPEYMYDASIKHGVKGIVYAGMGAGSVSKRGDAGIRKAESKGIVVVRSSRTGSGIVPPDAGQPGLVADSLSPAKSRILLMLALTKTTNPAVIQDYFHAY

SEQ ID NO: 6 - protein, 337 aa, Pseudomonas sp.
KEVENQQKLANVVILATGGTIAGAGASAANSATYQAAKVGVDKLIAGVPELADLANVRGEQVMQIASESITNDDLLKLGKRVAELADSNDVDGIVITHGTDTLEETAYFLDLTLNTDKPIVVVGSMRPGTAMSADGMLNLYNAVAVASNKDSRGKGVLVTMNDEIQSGRDVSKSINIKTEAFKSAWGPLGMVVEGKSYWFRLPAKRHTVNSEFDIKQISSLPQVDIAYSYGNVTDTAYKALAQNGAKALIHAGTGNGSVSSRLTPALQTLRKTGTQIIRSSHVNQGGFVLRNAEQPDDKNDWVVAHDLNPEKARILVELAMVKTQDSKELQRIFWEY

SEQ ID NO: 7 - protein, 331 aa, Acinetobacter glutaminasificans
KNNVVIVATGGTIAGAGASSTNSATYSAAKVPVDALIKAVPQVNDLANITGIQALQVASESITDKELLSLARQVNDLVKKPSVNGVVITHGTDTMEETAFFLNLVVHTDKPIVLVGSMRPSTALSADGPLNLYSAVALASSNEAKNKGVMVLMNDSIFAARDVTKGINIHTHAFVSQWGALGTLVEGKPYWFRSSVKKHTNNSEFNIEKIQGDALPGVQIVYGSDNMMPDAYQAFAKAGVKAIIHAGTGNGSMANYLVPEVRKLHDEQGLQIVRSSRVAQGFVLRNAEQPDDKYGWIAAHDLNPQKARLLMALALTKTNDAKEIQNMFWNY

SEQ ID NO: 8 - protein, 330 aa, Wolinella succinogenes
MAKPQVTILATGGTIAGSGESSVKSSYSAGAVTVDKLLAAVPAINDLATIKGEQISSIGSQEMTGKVWLKLAKRVNELLAQKETEAVIITHGTDTMEETAFFLNLTVKSQKPVVLVGAMRSGSSMSADGPMNLYNAVNVAINKASTNKGVVIVMNDEIHAAREATKLNTTAVNAFASPNTGKIGTVYYGKVEYFTQSVRPHTLASEFDISKIEELPRVDILYAHPDDTDVLVNAALQAGAKGIIHAGMGNGNPFPLTQNALEKAAKSGVVVARSSRVGSGSTTQEAEVDDKKLGFVATESLNPQKARVLLMLALTKTSDREAIQKIFSTY

SEQ ID NO: 9 - protein, 328 aa, Homo sapiens
MMSGEPLHVKTPIRDSMALSKMAGTSVYLKMDSAQPSGSFKIRGIGHFCKRWAKQGCAHFVCSSAGNAGMAAAYAARQLGVPATIVVPSTTPALTIERLKNEGATVKVVGELLDEAFELAKALAKNNPGWVYIPPFDDPLIWEGHASIVKELKETLWEKPGAIALSVGGGGLLCGVVQGLQEVGWGDVPVIAMETFGAHSFHAATTAGKLVSLPKITSVAKALGVKTVGAQALKLFQEHPIFSEVISDQEAVAAIEKFVDDEKILVEPACGAALAAVYSHVIQKLQLEGNLRTPLPSLVVIVCGGSNISLAQLRALKEQLGMTNRLPK

SEQ ID NO: 10 - protein, 483 aa, Homo sapiens
MTMPVNGAHKDADLWSSHDKMLAQPLKDSDVEVYNIIKKESNRQRVGLELIASENFASRAVLEALGSCLNNKYSEGYPGQRYYGGTEFIDELETLCQKRALQAYKLDPQCWGVNVQPYSGSPANFAVYTALVEPHGRIMGLDLPDGGHLTHGFMTDKKKISATSIFFESMPYKVNPDTGYINYDQLEENARLFHPKLIIAGTSCYSRNLEYARLRKIADENGAYLMADMAHISGLVAAGVVPSPFEHCHVVTTTTHKTLRGCRAGMIFYRKGVKSVDPKTGKEILYNLESLINSAVFPGLQGGPHNHAIAGVAVALKQAMTLEFKVYQHQVVANCRALSEALTELGYKIVTGGSDNHLILVDLRSKGTDGGRAEKVLEACSIACNKNTCPGDRSALRPSGLRLGTPALTSRGLLEKDFQKVAHFIHRGIELTLQIQSDTGVRATLKEFKERLAGDKYQAAVQALREEVESFASLFPLPGLPDF

SEQ ID NO: 11 - protein, 322 aa, Homo sapiens
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 12 - protein, 418 aa, Pseudomonas aeruginosa
MSTEKTKLGVHSEAGKLRKVMVCSPGLAHQRLTPSNCDELLFDDVIWVNQAKRDHFDFVTKMRERGIDVLEMHNLLTETIQNPEALKWILDRKITADSVGLGLTSELRSWLESLEPRKLAEYLIGGVAADDLPASEGANILKMYREYLGHSSFLLPPLPNTQFTRDTTCWIYGGVTLNPMYWPARRQETLLTTAIYKFHPEFANAEFEIWYGDPDKDHGSSTLEGGDVMPIGNGVVLIGMGERSSRQAIGQVAQSLFAKGAAERVIVAGLPKSRAAMHLDTVFSFCDRDLVTVFPEVVKEIVPFSLRPDPSSPYGMNIRREEKTFLEVVAESLGLKKLRVVETGGNSFAAEREQWDDGNNVVCLEPGVVVGYDRNTYTNTLLRKAGVEVITISASELGRGRGGGHCMTCPIVRDPIDY

SEQ ID NO: 13 - protein, 398 aa, Pseudomonas putida
MHGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 14 - protein, 567 aa, Homo sapiens
MAPLALHLLVLVPILLSLVASQDWKAERSQDPFEKCMQDPDYEQLLKVVTWGLNRTLKPQRVIVVGAGVAGLVAAKVLSDAGHKVTILEADNRIGGRIFTYRDQNTGWIGELGAMRMPSSHRILHKLCQGLGLNLTKFTQYDKNTWTEVHEVKLRNYVVEKVPEKLGYALRPQEKGHSPEDIYQMALNQALKDLKALGCRKAMKKFERHTLLEYLLGEGNLSRPAVQLLGDVMSEDGFFYLSFAEALRAHSCLSDRLQYSRIVGGWDLLPRALLSSLSGLVLLNAPVVAMTQGPHDVHVQIETSPPARNLKVLKADVVLLTASGPAVKRITFSPPLPRHMQEALRRLHYVPATKVFLSFRRPFWREEHIEGGHSNTDRPSRMIFYPPPREGALLLASYTWSDAAAAFAGLSREEALRLALDDVAALHGPVVRQLWDGTGVVKRWAEDQHSQGGFVVQPPALWQTEKDDWTVPYGRIYFAGEHTAYPHGWVETAVKSALRAAIKINSRKGPASDTASPEGHASDMEGQGHVHGVASSPSHDLAKEEGSHPPVQGQLSLQNTTHTRTSH

SEQ ID NO: 15 - protein, 384 aa, Escherichia coli
MAKHLFTSESVSEGHPDKIADQISDAVLDAILEQDPKARVACETYVKTGMVLVGGEITTSAWVDIEEITRNTVREIGYVHSDMGFDANSCAVLSAIGKQSPDINQGVDRADPLEQGAGDQGLMFGYATNETDVLMPAPITYAHRLVQRQAEVRKNGTLPWLRPDAKSQVTFQYDDGKIVGIDAVVLSTQHSEEIDQKSLQEAVMEEIIKPILPAEWLTSATKFFINPTGRFVIGGPMGDCGLTGRKIIVDTYGGMARHGGGAFSGKDPSKVDRSAAYAARYVAKNIVAAGLADRCEIQVSYAIGVAEPTSIMVETFGTEKVPSEQLTLLVREFFDLRPYGLIQMLDLLHPIYKETAAYGHFGREHFPWEKTDKAQLLRDAAGLK

SEQ ID NO: 16 - protein, 405 aa, Homo sapiens
MQEKDASSQGFLPHFQHFATQAIHVGQDPEQWTSRAVVPPISLSTTFKQGAPGQHSGFNYSRSGNPTRNCLEKAVAALDGAKYCLAFASGLAATVTITHLLKAGDQIICMDDVYGGTNLYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAVSLGGFESLAELPAIMTHASVLKNDRDVLGISDTLIRLSVGLEDEEDLLEDLDQALKAAHPPSGSHS

SEQ ID NO: 17 - protein, 298 aa, Pseudomonas aeruginosa
MKQIAFIGLGHMGAPMATNLLKAGYLLNVFDLVQSAVDGLVAAGASAARSARDAVQGADVVISMLPASQHVEGLYLDDDGLLAHIAPGTLVLECSTIAPTSARKIHAAARERGLAMLDAPVSGGTAGAAAGTLTFMVGGDAEALEKARPLFEAMGRNIFHAGPDGAGQVAKVCNNQLLAVLMIGTAEAMALGVANGLEAKVLAEIMRRSSGGNWALEVYNPWPGVMENAPASRDYSGGFMAQLMAKDLGLAQEAAQASASSTPMGSLALSLYRLLLKQGYAERDFSVVQKLFDPTQGQ

SEQ ID NO: 18 - protein, 403 aa, Homo sapiens [sequence omitted]

SEQ ID NO: 19 - protein, 567 aa, Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 20 - protein, 131 aa, Artificial Sequence
  Description of Artificial Sequence: Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 21 - protein, 9 aa, Artificial Sequence
  Description of Artificial Sequence: Synthetic peptide
YPYDVPDYA

SEQ ID NO: 22 - protein, 19 aa, Artificial Sequence
  Description of Artificial Sequence: Synthetic peptide
MYGKIIFVLLLSEIVSISA

SEQ ID NO: 23 - skipped

SEQ ID NO: 24 - protein, 24 aa, Artificial Sequence
  Description of Artificial Sequence: Synthetic peptide
ICYGTIAVIVFFLIGFMIGYLGYC

SEQ ID NO: 25 - skipped

SEQ ID NO: 26 - protein, 79 aa, Artificial Sequence
  Description of Artificial Sequence: Synthetic polypeptide
MEGGDQSEEEPRERSQAGGMGTLWSQESTPEERLPVEGSRPWAVARRVLTAILILGLLLCFSVLLFYNFQNCGPRPCET

SEQ ID NO: 27 - protein, 78 aa, Artificial Sequence
  Description of Artificial Sequence: Synthetic polypeptide
MQPQESHVHYSRWEDGSRDGVSLGAVSSTEEASRCRRISQRLCTGKLGIAMKVLGGVALFWIIFILGYLTGYYVHKCK

SEQ ID NO: 28 - protein, 27 aa, Artificial Sequence
  Description of Artificial Sequence: Synthetic peptide
GGGGSGGGGYPYDVPDYAGGGGSGGGG

SEQ ID NO: 29 - protein, 26 aa, Artificial Sequence
  Description of Artificial Sequence: Synthetic peptide
GGGGSGRGGSGRGGSGRGGSGRGGRR

SEQ ID NO: 30 - protein, 29 aa, Artificial Sequence
  Description of Artificial Sequence: Synthetic peptide
GEGGSGEGGSYPYDVPDYAGEGGSGEGGS

SEQ ID NO: 31 - protein, 29 aa, Artificial Sequence
  Description of Artificial Sequence: Synthetic peptide
GGGGSGGGGSYPYDVPDYAGGGGSGGGGS

SEQ ID NO: 32 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 33 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 34 - protein, 456 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLLKLENKEAKVSKREKAVWVLNPEAGMWQCLLSDSGQVLLSNIKVLPTWSTPVQPMALIVLGGVAGLLLFIGLGIFFCVRCRHRRRQAERMSQIKRLLSEKKTCQCPHRFQKTCSPI

SEQ ID NO: 35 - protein, 132 aa, Homo sapiens
  MOD_RES (13)..(13): Any amino acid
  MOD_RES (97)..(97): Any amino acid
  MOD_RES (99)..(99): Any amino acid
  MOD_RES (106)..(106): Any amino acid
  MOD_RES (108)..(108): Any amino acid
  MOD_RES (110)..(110): Any amino acid
  MOD_RES (113)..(113): Any amino acid
  MOD_RES (121)..(121): Any amino acid
QNEYFDSLLHACXFCQLRCSSNTPPLTCQRYCNASVTNSVKGTNAILWTCLGLSLIISLAVFVLMFLLRKISSEPLKDEFKNTEMESHSVAQAGVQXRXLNSLQPXPXGXKQXSHLSLLSXPDYRIRSPGHG

SEQ ID NO: 36 - protein, 220 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 37 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 38 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 39 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 40 - protein, 242 aa, Artificial Sequence
  Description of Artificial Sequence: Synthetic polypeptide
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVK

SEQ ID NO: 41 - protein, 242 aa, Artificial Sequence
  Description of Artificial Sequence: Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 42 - protein, 242 aa, Artificial Sequence; identical to SEQ ID NO: 40
  Description of Artificial Sequence: Synthetic polypeptide

SEQ ID NO: 43 - protein, 337 aa, Pseudomonas sp.
KEVENQQKLANVVILATGGTIAGAGASAANSATYQAAKVGVDKLIAGVPELADLANVRGEQVMQIASESITNDDLLKLGKRVAELADSNDVDGIVITHGTDTLEETAYFLNLVEKTDKPIVVVGSMRPGTAMSADGMLNLYNAVAVASNKDSRGKGVLVTMNDEIQSGRDVSKSINIKTEAFKSAWGPLGMVVEGKSYWFRLPAKRHTVNSEFDIKQISSLPQVDIAYSYGNVTDTAYKALAQNGAKALIHAGTGNGSVSSRVVPALQELRKNGVQIIRSSHVNQGGFVLRNAEQPDDKNDWVVAHDLNPQKARILAMVAMTKTQDSKELQRIFWEY

SEQ ID NO: 44 - protein, 669 aa, Homo sapiens
MMRLRGSGMLRDLLLRSPAGVSATLRRAQPLVTLCRRPRGGGRPAAGPAAAARLHPWWGGGGWPAEPLARGLSSSPSEILQELGKGSTHPQPGVSPPAAPAAPGPKDGPGETDAFGNSEGKELVASGENKIKQGLLPSLEDLLFYTIAEGQEKIPVHKFITALKSTGLRTSDPRLKECMDMLRLTLQTTSDGVMLDKDLFKKCVQSNIVLLTQAFRRKFVIPDFMSFTSHIDELYESAKKQSGGKVADYIPQLAKFSPDLWGVSVCTVDGQRHSTGDTKVPFCLQSCVKPLKYAIAVNDLGTEYVHRYVGKEPSGLRFNKLFLNEDDKPHNPMVNAGAIVVTSLIKQGVNNAEKFDYVMQFLNKMAGNEYVGFSNATFQSERESGDRNFAIGYYLKEKKCFPEGTDMVGILDFYFQLCSIEVTCESASVMAATLANGGFCPITGERVLSPEAVRNTLSLMHSCGMYDFSGQFAFHVGLPAKSGVAGGILLVVPNVMGMMCWSPPLDKMGNSVKGIHFCHDLVSLCNFHNYDNLRHFAKKLDPRREGGDQRVKSVINLLFAAYTGDVSALRRFALSAMDMEQRDYDSRTALHVAAAEGHVEVVKFLLEACKVNPFPKDRWNNTPMDEALHFGHHDVFKILQEYQVQYTPQGDSDNGKENQTVHKNLDGLL

SEQ ID NO: 45 - protein, 196 aa, Bacillus subtilis
MLTIGVLGLQGAVREHIHAIEACGAAGLVVKRPEQLNEVDGLILPGGESTTMRRLIDTYQFMEPLREFAAQGKPMFGTCAGLIILAKEIAGSDNPHLGLLNVVVERNSFGRQVDSFEADLTIKGLDEPFTGVFIRAPHILEAGENVEVLSEHNGRIVAAKQGQFLGCSFHPELTEDHRVTQLFVEMVEEYKQKALV

SEQ ID NO: 46 - protein, 310 aa, Escherichia coli
MLDANKLQQAVDQAYTQFHSLNGGQNADYIPFLANVPGQLAAVAIVTCDGNVYSAGDSDYRFALESISKVCTLALALEDVGPQAVQDKIGADPTGLPFNSVIALELHGGKPLSPLVNAGAIATTSLINAENVEQRWQRILHIQQQLAGEQVALSDEVNQSEQTTNFHNRAIAWLLYSAGYLYCDAMEACDVYTRQCSTLLNTIELATLGATLAAGGVNPLTHKRVLQADNVPYILAEMMMEGLYGRSGDWAYRVGLPGKSGVGGGILAVVPGVMGIAAFSPPLDEDGNSVRGQKMVASVAKQLGYNVFKG

SEQ ID NO: 47 - protein, 602 aa, Mus musculus
MRSMRALQNALSRAGSHGRRGGWGHPSRGPLLGRGVRYYLGEAAAQGRGTPHSHQPQHSDHDASHSGMLPRLGDLLFYTIAEGQERIPIHKFTTALKATGLQTSDPRLQDCMSKMQRMVQESSSGGLLDRELFQKCVSSNIVLLTQAFRKKFVIPDFEEFTGHVDRIFEDAKEPTGGKVAAYIPHLAKSNPDLWGVSLCTVDGQRHSVGHTKIPFCLQSCVKPLTYAISVSTLGTDYVHKFVGKEPSGLRYNKLSLNEEGIPHNPMVNAGAIVVSSLIKMDCNKAEKFDFVLQYLNKMAGNEFMGFSNATFQSEKETGDRNYAIGYYLKEKKCFPKGVDMMAALDLYFQLCSVEVTCESGSVMAATLANGGICPITGESVLSAEAVRNTLSLMHSCGMYDFSGQFAFHVGLPAKSAVSGAILLVVPNVMGMMCLSPPLDKLGNSQRGINFCQKLVSLFNFHNYDNLRHCARKLDPRREGGEVRNKTVVNLLFAAYSGDVSALRRFALSAMDMEQKDYDSRTALHVAAAEGHIEVVKFLIEACKVNPFVKDRWGNIPLDDAVQFNHLEVVKLLQDYHDSYLLSETQAEAAAETLSKENLESMV

SEQ ID NO: 48 - protein, 227 aa, Bacillus subtilis
MKFAVIVLPGSNCDIDMYHAVKDELGHEVEYVWHEETSLDGFDGVLIPGGFSYGDYLRCGAIARFANIMPAVKQAAAEGKPVLGVCNGFQILQELGLLPGAMRRNKDLKFICRPVELIVQNDETLFTASYEKGESITIPVAHGEGNFYCDDETLATLKENNQIAFTYGSNINGSVSDIAGVVNEKGNVLGMMPHPERAVDELLGSADGLKLFQSIVKNWRETHVTTA

SEQ ID NO: 49 - protein, 690 aa, Aspergillus oryzae
MMHFLSFCLSVASLVSYAGAASTFSPARPPALPLAVKSPYLSTWLSAGTDGGNGGYLAGQWPTFWFGQVTGWAGQIRVDNSTYTWMGAIPNTPTVNQTSFEYTSTSSVFTMRVGDMVEMKVKFLSPITPDDLRRQSLVFSYLDVDVESIDGKAHDIQVYADISAEWASGDRNAIAQWDYGVTDDGVAYHKVYRQTQLLFSENTEQAEWGEWYWATDDQDGLSYQSGPDVDVRGAFAKNGKLANSDDKNYRAISTNWPVFAFSRDLGSVKTSAGTLFSIGLAQDSAIQYSGKPEGTTVMPSLWKSYFSTATAALEFFHHDYAAAAALSKDLDDRISKDSIDAAGQDYLTITSLTVRQVFAAVQLTGTPEDPYIFMKEISSNGNMNTVDVIFPAHPIFLYTNPELLKLILKPIYEIQENGKYPNTYAMHDIGTHYPNATGHPKGDDEKMPLEECGNMVIMALAYAQKAKDNDYLSQHYPILNKWTTYLVEDSIYPANQISTDDFAGSLANQTNLALKGIIGIQAMAVISNTTGHPDDASNHSSIAKDYIARWQTLGVAHDANPPHTTLSYGANETHGLLYNLYADRELGLNLVPQSVYDMQNTFYPTVKEKYGVPLDTRHVYTKADWELFTAAVASESVRDMFHQALATWINETPTNRAFTDLYDTQTGNYPAGITFIARPVMGGAFALLIL

SEQ ID NO: 50 - protein, 213 aa, Thermotoga maritima
MKPRACVVVYPGSNCDRDAYHALEINGFEPSYVGLDDKLDDYELIILPGGFSYGDYLRPGAVAAREKIAFEIAKAAERGKLIMGICNGFQILIEMGLLKGALLQNSSGKFICKWVDLIVENNDTPFTNAFEKGEKIRIPIAHGFGRYVKIDDVNVVLRYVKDVNGSDERIAGVLNESGNVFGLMPHPERAVEELIGGEDGKKVFQSILNYLKR

SEQ ID NO: 51 - protein, 331 aa, Acinetobacter glutaminasificans; identical to SEQ ID NO: 7

SEQ ID NO: 52 - protein, 362 aa, Pseudomonas putida
MNAALKTFAPSALALLLILPSSASAKEAETQQKLANVVILATGGTIAGAGASAANSATYQAAKLGVDKLIAGVPELADIANVRGEQVMQIASESISNDDLLKLGKRVAELAESKDVDGIVITHGTDTLEETAFFLNLVEKTDKPIVVVGSMRPGTAMSADGMLNLYNAVAVASDKQSRGKGVLVTMNDEIQSGRDVSKAVNIKTEAFKSAWGPMGMVVEGKSYWFRLPAKRHTVNSEFDIKQISSLPQVDIAYGYGNVTDTAYKALAQNGAKALIHAGTGNGSVSSRVVPALQELRKNGVQIIRSSHVNQGGFVLRNAEQPDDKNDWVVAHDLNPQKARILAMVAMTKTQDSKELQRIFWEY

SEQ ID NO: 53 - protein, 244 aa, Artificial Sequence
  Description of Artificial Sequence: Synthetic polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 54 - protein, 435 aa, Artificial Sequence
  Description of Artificial Sequence: Synthetic polypeptide
MQPQESHVHYSRWEDGSRDGVSLGAVSSTEEASRCRRISQRLCTGKLGIAMKVLGGVALFWIIFILGYLTGYYVHKCKGGGGSGGGGSGGGGSGGGGSGGGGSGGGGSADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 55 - protein, 421 aa, Artificial Sequence
  Description of Artificial Sequence: Synthetic polypeptide
MYGKIIFVLLLSEIVSISAEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKGGGGSGGGGSGGGGSGGGGSGGGGSGGGGLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 56 - protein, 450 aa, Artificial Sequence
  Description of Artificial Sequence: Synthetic polypeptide
EVQLVQSGAEVKKPGESLKISCKGSGYSFTDYYMKWARQMPGKGLEWMGDIIPSNGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 57 - protein, 220 aa, Artificial Sequence
  Description of Artificial Sequence: Synthetic polypeptide
DIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 117 aa, Artificial Sequence
  Description of Artificial Sequence: Synthetic polypeptide
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 59 - protein, 112 aa, Artificial Sequence
  Description of Artificial Sequence: Synthetic polypeptide
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 60 - protein, 121 aa, Artificial Sequence
  Description of Artificial Sequence: Synthetic polypeptide
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 61 - protein, 108 aa, Artificial Sequence
  Description of Artificial Sequence: Synthetic polypeptide
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

(19) United States
(12) Patent Application Publication
(10) Pub. No.: US 2019/0160102 A1
(43) Pub. Date: May 30, 2019

(54) COMPOSITIONS AND METHODS RELATED TO THERAPEUTIC CELL SYSTEMS FOR TUMOR GROWTH INHIBITION

(71) Applicant: RUBIUS THERAPEUTICS, INC., Cambridge, MA (US)

(72) Inventors: Lenka Hoffman, Malden, MA (US); Tom Wickham, Groton, MA (US); Nathan Dowden, Winchester, MA (US); Torben Straight Nissen, Chestnut Hill, MA (US); Kristian Eric Teichert, Methuen, MA (US)

(21) Appl. No.: 16/179,727

(22) Filed: Nov. 2, 2018

Related U.S. Application Data
(60) Provisional application No. 62/581,536, filed on Nov. 3, 2017.

(51) Int. Cl. A61K 35/18; C12N 9/82; C07K 16/28; A61P 35/00; A61P 35/02; G01N 33/50; C12N 5/078

(52) U.S. Cl. CPC A61K 35/18; A61K 2039/505; C12Y 305/01001; C07K 16/2803; A61P 35/00; A61P 35/02; G01N 33/5044; C12N 5/0641; C07K 2319/03; C07K 2319/30; C07K 2319/74; C07K 2317/565; C07K 2317/622; C07K 2317/76; C07K 2319/21; C12N 9/82

(57) ABSTRACT

The disclosure provides, e.g., enucleated erythroid cells comprising an amino acid degradative enzyme such as asparaginase and a targeting moiety such as an anti-CD33 antibody molecule. The cells may be used, e.g., to treat cancers such as AML.

### RELATED APPLICATIONS

This application claims priority to U.S. Ser. No. 62/581,536 filed Nov. 3, 2017, the contents of which are incorporated herein by reference in their entirety.

### BACKGROUND

Purified amino acid degradative enzymes have been tested for the ability to starve cancer cells of essential amino acids. However, administration of the enzymes directly to subjects can lead to toxicity in non-cancerous cells, and some enzymes may be immunogenic leading to undesirable clinical reactions. There is a need in the art for additional methods for delivering amino acid-degradative enzymes to subjects for therapeutic applications, such as cancer therapies.

### SUMMARY OF THE INVENTION

The disclosure provides, e.g., enucleated erythroid cells comprising an amino acid degradative enzyme such as an asparaginase molecule and a targeting moiety such as an anti-CD33 antibody molecule. The cells may be used, e.g., to treat cancers such as an acute myeloid leukaemia (AML).

The present disclosure provides, in some aspects, a genetically engineered erythroid cell (e.g., enucleated erythroid cell) comprising:

a first exogenous polypeptide comprising an amino acid degradative enzyme; and

a second exogenous polypeptide comprising a cell targeting moiety.

The present disclosure provides, in some aspects, a genetically engineered erythroid cell comprising an exogenous polypeptide that has glutaminase-degrading activity.

The present disclosure provides, in some aspects, a genetically engineered erythroid cell (e.g., enucleated erythroid cell) comprising:

a first exogenous polypeptide comprising an asparaginase molecule; and

a second exogenous polypeptide comprising an anti-CD33 cell targeting moiety.

In some aspects, the disclosure provides a composition, e.g., a pharmaceutical composition, comprising genetically engineered, enucleated erythroid cells described herein. In some embodiments, the pharmaceutical composition comprises a pharmaceutically acceptable excipient.

In some aspects, the disclosure provides a device comprising: a) a container (e.g., a vial or syringe), and b) a plurality of genetically engineered erythroid cells described herein.

In some aspects, the disclosure provides a method of treating a cancer, comprising administering to a subject in need thereof a therapeutically effective amount of the genetically engineered, enucleated erythroid cells described here. Similarly, in some aspects, the disclosure provides the use of a cell or composition described herein in the manufacture of a medicament for treating a disease, e.g., a cancer. In related aspects, the disclosure comprises a cell or composition of cells described herein, for use in treating a disease, e.g., a cancer.

In some aspects, the disclosure provides a method of treating a cancer, comprising administering to a subject in need thereof a therapeutically effective amount of the genetically engineered erythroid cells (e.g., enucleated erythroid cells) comprising an exogenous polypeptide that has glutaminase-degrading activity. Similarly, in some aspects, the disclosure provides the use of a plurality of erythroid cells (e.g., enucleated erythroid cells) comprising an exogenous polypeptide that has glutaminase-degrading activity, in the manufacture of a medicament for treating a disease, e.g., a cancer. In related aspects, the disclosure comprises a plurality of erythroid cells (e.g., enucleated erythroid cells) comprising an exogenous polypeptide that has glutaminase-degrading activity, for use in treating a disease, e.g., a cancer.

In some aspects, the disclosure provides a method of targeting an amino acid degradative enzyme or polypeptide comprising an amino acid degradative enzyme to a target cell in a subject, comprising administering a composition of genetically engineered, enucleated erythroid cells described herein to the subject. Similarly, in some aspects, the disclosure provides the use of a cell or composition described herein in the manufacture of a medicament for targeting an amino acid degradative enzyme to a target cell. In related aspects, the disclosure comprises a cell or composition of cells described herein, for use in targeting an amino acid degradative enzyme to a target cell.

In some aspects, the disclosure provides a method of reducing the concentration of an amino acid in a subject, e.g., locally reducing the concentration of an amino acid at a target cell in the subject, comprising administering a composition of genetically engineered, enucleated erythroid cells described herein to the subject. In some embodiments, the amino acid concentration is reduced in the blood, plasma, or serum of the subject. In some embodiments, the amino acid concentration is reduced in a tissue of the subject (e.g., a cancerous tissue (e.g., a tumor) or cancerous cell). In some embodiments, the amino acid concentration (e.g., the intracellular concentration of the amino acid) is reduced in a cell (e.g., a cancer cell) of the subject. Similarly, in some aspects, the disclosure provides the use of a cell or composition described herein in the manufacture of a medicament for reducing the concentration of an amino acid in a subject, e.g., locally reducing the concentration of an amino acid at a target cell in the subject. In related aspects, the disclosure comprises a cell or composition of cells described herein, for use in reducing the concentration of an amino acid in a subject, e.g., locally reducing the concentration of an amino acid at a target cell in the subject.

In some aspects, the disclosure provides a method of selecting a genetically engineered, enucleated erythroid cell for administration to a subject having a cancer, comprising:

(a) acquiring information about the sensitivity of a cancer to an amino acid degradative enzyme, e.g., determining whether the cancer is sensitive to one or more amino acid degradative enzymes;

(b) responsive to (a), selecting a genetically engineered, enucleated erythroid cell comprising an amino acid degradative enzyme to which the cancer is sensitive; and

(c) optionally, administer the genetically engineered, enucleated erythroid cell comprising the amino acid degradative enzyme to the subject.

In some aspects, the disclosure provides a method of selecting a genetically engineered, enucleated erythroid cell for administration to a subject having a cancer, comprising:

(a) acquiring information about a cell surface marker of the cancer, e.g., determining whether one or more cell surface marker is present on the cancer;

(b) responsive to (a), selecting a genetically engineered, enucleated erythroid cell comprising a cell targeting moiety that binds the cell surface marker; and

(c) optionally, administering the genetically engineered, enucleated erythroid cell comprising the cell targeting moiety to the subject.

In some aspects, the disclosure provides a method of selecting a genetically engineered, enucleated erythroid cell for administration to a subject having a leukemia, e.g. AML, comprising:

(a) acquiring information about whether cancer comprises CD33, e.g., determining whether CD33 is present on the cancer;

(b) responsive to (a), selecting a genetically engineered, enucleated erythroid cell comprising an anti-CD33 cell targeting moiety; and

(c) optionally, administering the genetically engineered, enucleated erythroid cell to the subject,

wherein the genetically engineered, enucleated erythroid cell comprises an asparaginase molecule, e.g., an asparaginase molecule having glutamine-degrading activity.

In some aspects, the disclosure provides a method of selecting a genetically engineered, enucleated erythroid cell for administration to a subject having a cancer, comprising:

(a) acquiring information about the sensitivity of a cancer to an amino acid degradative enzyme, e.g., determining whether the cancer is sensitive to one or more amino acid degradative enzymes;

(b) acquiring information about a cell surface marker of the cancer, e.g., determining whether one or more cell surface marker is present on the cancer;

(c) responsive to (a), selecting a genetically engineered, enucleated erythroid cell comprising an amino acid degradative enzyme to which the cancer is sensitive and further comprising a cell targeting moiety that binds the cell surface marker; and

(d) optionally, administer the genetically engineered, enucleated erythroid cell comprising the amino acid degradative enzyme and the cell targeting moiety to the subject.

In some aspects, the disclosure provides an erythroid cell, e.g., a genetically engineered, enucleated erythroid cell comprising an exogenous fusion polypeptide which comprises SMIM or a fragment thereof, e.g., a transmembrane fragment thereof, or a sequence having at least 70%, 80%, 85%, 90%, 95%, 96%, 97%, 98%, or 99% identity to a SMIM polypeptide of SEQ ID NO: 27, or a transmembrane fragment thereof.

Any of the aspects herein, e.g., the erythroid cell compositions and methods above, can be combined with one or more of the embodiments herein, e.g., an embodiment below.

In some embodiments, the amino acid degradative enzyme comprises an asparaginase molecule.

In some embodiments, the amino acid degradative enzyme comprises an enzymatically active polypeptide having at least 70%, 80%, 85%, 90%, 95%, 96%, 97%, 98%, or 99% identity to the amino acid sequence of any of SEQ ID NOs: 116-129. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NO: 116. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NO: 117. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NO: 118. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NO: 119. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NO: 120. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NO: 121. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NO: 122. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NO: 123. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NO: 124. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NO: 125. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NO: 126. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NO: 127. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NO: 128. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NO: 129.

In some embodiments, the asparaginase molecule has one or more of:

an asparaginase activity with a Kₘ of 0.004-0.01, 0.01-0.02, 0.02-0.05, or 0.05-0.1 mM, or less than 0.1, 0.05, 0.02, 0.01, or 0.005 mM (e.g., down to about 0.004 mM);

an asparaginase activity with a K_{cat} of 20-50, 50-100, 100-200, 200-500, 500-1000, or 1000-1500, s⁻¹, or at least 20, 50, 100, 200, 500, or 100 (e.g., up to about 1000 or 1500) s⁻¹;

a glutaminase activity with a Kₘ of 0.002-0.005, 0.005-0.01, 0.01-0.02, 0.02-0.05, 0.05-0.1, 0.1-0.2, 0.2-0.5, 0.5-1, 1-2, 2-5, 5-10, 10-20, 20-50, or 50-100 mM; or

a gluatminase activity with a K_{cat} of 0.2-0.5, 0.5-1, 1-2, 2-5, 5-10, 10-20, 20-50, or 50-100 s⁻¹.

In some embodiments, the asparaginase molecule has one or more of:

an asparagine-degrading activity with a Kₘ of 0.004-0.01, 0.01-0.02, 0.02-0.05, or 0.05-0.1 mM, or less than 0.1, 0.05, 0.02, 0.01, or 0.005 mM (e.g., down to about 0.004 mM);

an asparagine-degrading activity with a K_{cat} of 20-50, 50-100, 100-200, 200-500, 500-1000, or 1000-1500, s⁻¹, or at least 20, 50, 100, 200, 500, or 100 (e.g., up to about 1000 or 1500) s⁻¹;

a glutamine-degrading activity with a Kₘ of 0.002-0.005, 0.005-0.01, 0.01-0.02, 0.02-0.05, 0.05-0.1, 0.1-0.2, 0.2-0.5, 0.5-1, 1-2, 2-5, 5-10, 10-20, 20-50, or 50-100 mM; or

a glutamine-degrading activity with a K_{cat} of 0.2-0.5, 0.5-1, 1-2, 2-5, 5-10, 10-20, 20-50, or 50-100 s⁻¹.

In some embodiments, the amino acid degradative enzyme has one or more of:

an amino acid-degrading activity with a Kₘ of 0.002-0.005, 0.004-0.01, 0.01-0.02, 0.02-0.05, or 0.05-0.1, 0.1-0.2, 0.2-0.5, 0.5-1, 1-2, 2-5, 5-10, 10-20, 20-50, or 50-100 mM, or less than 0.1, 0.05, 0.02, 0.01, or 0.005 mM (e.g., down to about 0.004 mM);

an amino acid-degrading activity with a K_{cat} of 0.2-0.5, 0.5-1, 1-2, 2-5, 5-10, 10-20, 20-50, 50-100, 100-200, 200-500, 500-1000, or 1000-1500, s⁻¹, or at least 20, 50, 100, 200, 500, or 100 (e.g., up to about 1000 or 1500) s⁻¹.

In some embodiments, the cell comprises at least 1×10⁻⁸, 2×10⁻⁸, 5×10⁻⁸, 1×10⁻⁹, 2×10⁻⁹, 5×10⁻⁹, 1×10⁻¹⁰, 2×10⁻¹⁰, 5×10⁻¹°, 1×10⁻¹¹, 2×10⁻¹¹, or 5×10⁻¹¹ units of asparaginase.

In some embodiments, the amino acid degradative enzyme comprises an asparaginase molecule having at least 70%, 80%, 85%, 90%, 95%, 96%, 97%, 98%, or 99% identity to the amino acid sequence of any of SEQ ID NOs: 3-8. In some embodiments, the amino acid degradative enzyme comprises an asparaginase molecule having at least 70%, 80%, 85%, 90%, 95%, 96%, 97%, 98%, or 99% identity to the amino acid sequence of SEQ ID NO: 3. In some embodiments, the amino acid degradative enzyme comprises an asparaginase molecule having at least 70%, 80%, 85%, 90%, 95%, 96%, 97%, 98%, or 99% identity to the amino acid sequence of SEQ ID NO: 4. In some embodiments, the amino acid degradative enzyme comprises an asparaginase molecule having at least 70%, 80%, 85%, 90%, 95%, 96%, 97%, 98%, or 99% identity to the amino acid sequence of SEQ ID NO: 5. In some embodiments, the amino acid degradative enzyme comprises an asparaginase molecule having at least 70%, 80%, 85%, 90%, 95%, 96%, 97%, 98%, or 99% identity to the amino acid sequence of SEQ ID NO: 6. In some embodiments, the amino acid degradative enzyme comprises an asparaginase molecule having at least 70%, 80%, 85%, 90%, 95%, 96%, 97%, 98%, or 99% identity to the amino acid sequence of SEQ ID NO: 7. In some embodiments, the amino acid degradative enzyme comprises an asparaginase molecule having at least 70%, 80%, 85%, 90%, 95%, 96%, 97%, 98%, or 99% identity to the amino acid sequence of SEQ ID NO: 8. In some embodiments, the amino acid degradative enzyme comprises an asparaginase molecule having at least 70%, 80%, 85%, 90%, 95%, 96%, 97%, 98%, or 99% identity to the amino acid sequence of SEQ ID NO: 68. In some embodiments, the amino acid degradative enzyme comprises an asparaginase molecule having at least 70%, 80%, 85%, 90%, 95%, 96%, 97%, 98%, or 99% identity to the amino acid sequence of SEQ ID NO: 69. In some embodiments, the amino acid degradative enzyme comprises an asparaginase molecule having at least 70%, 80%, 85%, 90%, 95%, 96%, 97%, 98%, or 99% identity to the amino acid sequence of SEQ ID NO: 70.

In some embodiments, the amino acid degradative enzymes form a dimer, trimer, tetramer, pentamer, or hexamer. In some embodiments, the erythroid cell comprises one or more dimer, trimer, tetramer, pentamer, or hexamer, of the amino acid degradative enzyme. In some embodiments, the cell comprises at least 1,000, 5,000, 10,000, 15,000, 20,000, 25,000, or 30,000 copies of the amino acid degradative enzyme. In some embodiments, the cell comprises at least about 1,000, about 5,000, about 10,000, about 15,000, about 20,000, about 25,000, or about 30,000 copies of the first exogenous polypeptide. In some embodiments, the erythroid cell comprises the amino acid degradative enzyme (e.g., an asparaginase molecule, wherein optionally the enzyme is at the surface of the cell) at a level of less than 40,000, 35,000, 30,000, 25,000, or 20,000 copies per cell. In some embodiments, the erythroid cell comprises 500-40,000, 500-35,000, 500-30,000, 500-25,000, 500-20,000 1,000-40,000, 1,000-35,000, 1,000-30,000, 1,000-25,000, or 1,000-20,000 copies of the amino acid degradative enzyme, e.g., an asparaginase molecule, wherein optionally the enzyme is at the surface of the cell. In some embodiments, the amino acid degradative enzyme at the surface of a cell is present at a level that does not induce an antibody titer against the enzyme that is greater than the antibody titer resulting from administration of an otherwise similar control cell that lacks the amino acid degradative enzyme. In some embodiments, the amino acid degradative enzyme at the surface of a cell is present at a level that induces an antibody titer against the enzyme that no more than 5%, 10%, 15%, 20%, 30%, 40%, 50%, 75%, or 100% greater than the antibody titer resulting from administration of an otherwise similar control cell that lacks the amino acid degradative enzyme (e.g., measured after 30 days, e.g., in an assay of Example 6). In some embodiments, the amino acid degradative enzyme at the surface of a cell is present at a level that results in a lower antibody titer against the enzyme compared to the antibody titer resulting from administration of an otherwise similar control cell that lacks the amino acid degradative enzyme (e.g., measured after 30 days, e.g., in an assay of Example 6). In some embodiments, administration of the cell comprising the amino acid degradative enzyme induces tolerance to the enzyme in the subject.

In some embodiments, the erythroid cell comprises the amino acid degradative enzyme (e.g., an asparaginase molecule) in a sufficient amount such that, upon administration to a subject, the serum level of the amino acid (e.g., asparagine or glutamine) in the subject is below 60, 50, 40, 30, 20, or 10 urn, e.g., about 2, 4, 6, or 8 days after dosing. In some embodiments, the erythroid cell comprises the amino acid degradative enzyme (e.g., an asparaginase molecule) in a sufficient amount such that, upon administration to a subject, the serum level of the amino acid (e.g., asparagine or glutamine), e.g., measured at about 2, 4, 6, or 8 days after dosing, is less than 80%, 70%, 60%, 50%, 40%, 30%, 20%, or 10% of the level prior to dosing.

In some embodiments, the subject does not experience weight loss, e.g., when measured at 10, 20, or 30 days after administration of the erythroid cells. In some embodiments, the subject experiences weight loss of no more than 1%, 2%, 5%, or 10% of body mass, e.g., when measured at 10, 20, or 30 days after administration of the erythroid cells. In some embodiments, weight is measured at 10, 20, or 30 days after administration of the first dose in a multi-dose regimen of erythroid cells.

In some embodiments, the amino acid degradative enzyme is capable of degrading asparagine, serine, methionine, or arginine. In some embodiments, the amino acid is selected from asparagine, serine, methionine, or arginine. In some embodiments, the amino acid is an essential amino acid in humans (e.g., phenylalanine, valine, threonine, tryptophan, methionine, leucine, isoleucine, lysine, or histidine). In some embodiments, the amino acid is a conditionally essential amino acid in humans (e.g., arginine, cysteine, glycine, glutamine, proline, and tyrosine). In some embodiments, the amino acid is a dispensable amino acid in humans (e.g., alanine, aspartic acid, asparagine, glutamic acid, and/or serine). In some embodiments, the amino acid is other than tryptophan. In some embodiments, the amino acid does not comprise an aromatic group. In some embodiments, the amino acid is a branched chain amino acid (e.g., leucine, isoleucine, and valine).

In some embodiments, the amino acid degradative enzyme does not require a cofactor (e.g., a coenzyme or an inorganic ion) for substantial activity. In some embodiments, the amino acid degradative enzyme does not require heme for substantial activity. In some embodiments, the amino acid degradative enzyme does not inhibit immune cells. In some embodiments, the amino acid degradative enzyme does not induce T cell anergy.

In some embodiments, a pharmaceutical composition described herein does not comprise a cofactor (e.g., a coenzyme or an inorganic ion). In some embodiments, a pharmaceutical composition described herein does not comprise exogenous heme. In some embodiments, a pharmaceutical composition described herein does not inhibit immune cells. In some embodiments, a pharmaceutical composition described herein does not induce T cell anergy.

In some embodiments, the enucleated erythroid cell does not require a cofactor (e.g., a coenzyme or an inorganic ion) for substantial activity. In some embodiments, the enucleated erythroid cell does not require heme for substantial activity. In some embodiments, the enucleated erythroid cell does not inhibit immune cells. In some embodiments, the enucleated erythroid cell does not induce T cell anergy.

In some embodiments, the amino acid degradative enzyme is derived from a bacterial, or fungal, plant, or invertebrate enzyme. In some embodiments, the amino acid degradative enzyme is derived from a mammalian enzyme, e.g., a human enzyme. In some embodiments, the amino acid degradative enzyme is derived from other than a mammalian enzyme, or other than a human enzyme.

In some embodiments, the amino acid degradative enzyme comprises a serine dehydratase molecule, a serine hydroxymethyltransferase molecule, an arginase-1 molecule, an arginine deiminase molecule, an L-methionine gamma-lyase molecule, an L-amino-acid oxidase molecule, an S-adenosylmethionine synthase molecule, a cystathionine gamma-lyase molecule, a NAD-dependent L-serine dehydrogenase molecule, an indoleamine 2,3-dioxygenase molecule, or a phenylalanine ammonia lyase molecule. In some embodiments, the amino acid degradative enzyme comprises a polypeptide of Table 3, or an enzymatically active polypeptide having at least 70%, 80%, 85%, 90%, 95%, 96%, 97%, 98%, or 99% identity thereto.

In some embodiments, the amino acid degradative enzyme comprises a polypeptide of any one of SEQ ID NOs: 9-19 or an enzymatically active polypeptide having at least 70%, 80%, 85%, 90%, 95%, 96%, 97%, 98%, or 99% identity to the amino acid sequence of any one of SEQ ID NOs: 9-19. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NOs: 9. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NOs: 10. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NOs: 11. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NOs: 12. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NOs: 13. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NOs: 14. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NOs: 15. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NOs: 16. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NOs: 17. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NOs: 18. In some embodiments, the amino acid degradative enzyme comprises an enzymatically active polypeptide having at least 70%, 80%, 85%, 90%, 95%, 96%, 97%, 98%, or 99% identity to the amino acid sequence of SEQ ID NOs: 19.

A transmembrane domain can be used to anchor the amino acid degradative enzyme. In some embodiments, the amino acid degradative enzyme further comprises a transmembrane domain. In some embodiments, the transmembrane domain comprises a transmembrane region of a type 1 membrane protein (e.g., GPA, ICAM-4, CD329, CD147), type 2 membrane protein (e.g., CD71 or Kell), or type 3 membrane protein (e.g., GLUT1, Aquaporin 1, or Band 3). In some embodiments, the transmembrane domain comprises a transmembrane domain of a protein of Table 4, or a transmembrane polypeptide having at least 70%, 80%, 85%, 90%, 95%, 96%, 97%, 98%, or 99% identity thereto. In some embodiments, the transmembrane domain comprises a transmembrane domain of a protein or a transmembrane polypeptide having at least 70%, 80%, 85%, 90%, 95%, 96%, 97%, 98%, or 99% identity to the amino acid sequence of any one of SEQ ID NOs: 20, 26, or 27. In some embodiments, the transmembrane domain comprises a transmembrane region of Kell. In some embodiments, the amino acid degradative enzyme comprises a 79-amino acid N-terminal fragment of Kell, e.g., as shown in amino acids 1-79 of SEQ ID NO: 1. In some embodiments, the transmembrane domain is fused to the N-terminus of the amino acid degradative enzyme. In some embodiments, the transmembrane domain is fused to the C-terminus of the amino acid degradative enzyme.

Linkers can also be used. In some embodiments, a linker is disposed between the transmembrane domain and the amino acid degradative enzyme. In some embodiments, the first exogenous polypeptide comprises a first transmembrane domain and a second transmembrane domain, and optionally a linker disposed between the first transmembrane domain and second transmembrane domain. In some embodiments, the first transmembrane domain is a transmembrane domain of a type 1 membrane protein (e.g., GPA) and the second transmembrane domain is a transmembrane domain of a type 2 membrane protein (e.g., CD71 or Kell). In some embodiments, the linker comprises a poly-glycine poly-serine linker. In some embodiments, the linker comprises an amino acid sequence as set forth in Table 5, or an amino acid sequence having at least 70%, 80%, 85%, 90%, 95%, 96%, 97%, 98%, or 99% identity thereto. In some embodiments, the linker comprises an amino acid sequence of any one of SEQ ID NOs: 28-31 or a linker polypeptide having at least 70%, 80%, 85%, 90%, 95%, 96%, 97%, 98%, or 99% identity to the amino acid sequence of any one of SEQ ID NOs: 28-31.

In some embodiments, the amino acid degradative enzyme is on the surface of the erythroid cell. In some embodiments, the amino acid degradative enzyme is inside the erythroid cell. In some embodiments, the amino acid degradative enzyme is in the cytoplasm of the erythroid cell. In some embodiments, the amino acid degradative enzyme is associated with the cell membrane of the erythroid cell, and in other embodiments, the amino acid degradative enzyme is not associated with the cell membrane of the erythroid cell.

Various cell targeting moieties can be used. In some embodiments, the second exogenous polypeptide is a fusion polypeptide comprising a transmembrane domain and a cell targeting moiety. In some embodiments, the cell targeting moiety comprises an antibody molecule or a ligand or receptor-binding fragment thereof. In some embodiments, the cell targeting moiety comprises an antibody molecule, a ligand or a receptor-binding fragment or variant of the ligand, or a receptor or a ligand-binding fragment or variant of the receptor, wherein the antibody molecule, ligand or receptor specifically bind to a cell surface marker. In some embodiments, the antibody molecule comprises a single chain antibody, e.g., an scFv, (scFv)₂, nanobody, or camelid antibody.

In some embodiments, an erythroid cell comprising an amino acid degradative enzyme and a targeting moiety has a greater anti-cancer activity than an otherwise similar control erythroid cell lacking the targeting moiety. For instance, in some embodiments, upon administration of the erythroid cell having the targeting moiety to a subject having a tumor, the subject experiences a greater suppression of tumor growth than a subject treated with an otherwise similar dose of otherwise similar erythroid cells lacking the targeting moiety, e.g., using an assay of Example 8. In some embodiments, the erythroid cell comprising the targeting moiety has greater activity than the control cell lacking the targeting moiety, when the control cells are administered at a dose greater than the erythroid cells having the targeting moiety, e.g., wherein the greater dose comprises at least 10%, 20%, 30%, 40%, 50%, two times, or three times more units of asparaginase activity per dose.

In some embodiments, the cell targeting moiety binds (e.g., specifically binds) to a cell surface marker, e.g., a surface protein, of a target cell. In some embodiments, the target cell is a cancer cell. In some embodiments, the cancer cell is a leukemia cell, e.g., an AML cell.

In some embodiments, the cancer cell is selected from an acute myeloid leukaemia (AML) cell, an acute lymphoblastic leukaemia (ALL) cell, an anal cancer cell, a bile duct cancer cell, a bladder cancer cell, a bone cancer cell, a bowel cancer cell, a brain tumor cell, a breast cancer cell, a carcinoid cell, a cervical cancer cell, a choriocarcinoma cell, a chronic lymphocytic leukaemia (CLL) cell, a chronic myeloid leukaemia (CML) cell, a colon cancer cell, a colorectal cancer cell, an endometrial cancer cell, an eye cancer cell, a gallbladder cancer cell, a gastric cancer cell, a gestational trophoblastic tumor (GTT) cell, a hairy cell leukaemia cell, a head and neck cancer cell, a Hodgkin lymphoma cell, a kidney cancer cell, a laryngeal cancer cell, a liver cancer cell, a lung cancer cell, a lymphoma cell, a melanoma cell, a skin cancer cell, a mesothelioma cell, a mouth or oropharyngeal cancer cell, a myeloma cell, a nasal or sinus cancer cell, a nasopharyngeal cancer cell, a non-Hodgkin lymphoma (NHL) cell, an oesophageal cancer cell, an ovarian cancer cell, a pancreatic cancer cell, a penile cancer cell, a prostate cancer cell, a rectal cancer cell, a salivary gland cancer cell, a non-melanoma skin cancer cell, a soft tissue sarcoma cell, a stomach cancer cell, a testicular cancer cell, a thyroid cancer cell, a uterine cancer cell, a vaginal cancer cell, and a vulval cancer cell.

In some embodiments, the target cell (e.g., cancer cell) is other than a T cell. In some embodiments, the cell targeting moiety binds a target other than CD4. In some embodiments, the target cell (e.g., cancer cell) is CD4-negative.

In some embodiments, the cell targeting moiety comprises an antibody molecule that binds a protein listed in Table 6.

In some embodiments, the cell targeting moiety comprises an antibody molecule comprising six CDRs from an antibody molecule of Table 7, wherein CDRs are determined according to Kabat, Chothia, or a combination thereof. In some embodiments, cell targeting moiety comprises an antibody molecule having a VH domain and a VL domain from Table 7, or an antigen-binding polypeptide having at least 70%, 80%, 85%, 90%, 95%, 96%, 97%, 98%, or 99% identity thereto. In some embodiments, the cell targeting moiety comprises an antibody molecule having a VH domain and a VL domain from SEQ ID NO: 40 or 41, or an antigen-binding polypeptide having at least 70%, 80%, 85%, 90%, 95%, 96%, 97%, 98%, or 99% identity thereto.

In some embodiments, the cell comprises at least 1,000, 5,000, 10,000, 15,000, 20,000, 25,000, or 30,000 copies of the cell targeting moiety.

In some embodiments, the cell targeting moiety binds CD33, CD20, CD4, BCMA, PSA, CD269, CD123, CD47, BCMA, CD28, or CD38. In some embodiments, the cell targeting moiety binds CD33.

In some embodiments, the cell is a reticulocyte or mature erythrocyte. In some embodiments, the cell is substantially non-immunogenic.

In some embodiments, a composition of cells described herein has substantially the same pharmacokinetic profile on a second or subsequent administration (e.g., a second, third, fourth, or fifth administration) as on the first administration in the same subject, e.g., the half-life after the second or subsequent administration is within 10%, 20%, or 30% greater or less than the half-life after the first administration, e.g., in an assay measuring percentage of Cy5 cells in the blood, e.g., in an assay of Example 6. In some embodiments, a composition of cells described herein has substantially the same pharmacokinetic profile as an otherwise similar cell population lacking exogenous polypeptides, e.g., wherein the half-life of the genetically engineered, enucleated erythroid cells is within 10%, 20%, or 30% greater or less than the half-life of the otherwise similar cell population lacking exogenous polypeptides, e.g., in an assay measuring percentage of Cy5 cells in the blood, e.g., in an assay of Example 6. In some embodiments, a composition of cells described herein has a half-life of at least 3, 4, 5, 6, 7, 8, 9, or 10 days in circulation in a subject, e.g., in an assay measuring percentage of Cy5 cells in the blood, e.g., in an assay of Example 6.

In some embodiments, the cancer is a leukemia, e.g., acute myeloid leukaemia (AML). In some embodiments, the cancer is acute lymphoblastic leukaemia (ALL), anal cancer, bile duct cancer, bladder cancer, bone cancer, bowel cancer, brain tumors, breast cancer, cancer of unknown primary, cancer spread to bone, cancer spread to brain, cancer spread to liver, cancer spread to lung, carcinoid, cervical cancer, choriocarcinoma, chronic lymphocytic leukaemia (CLL), chronic myeloid leukaemia (CML), colon cancer, colorectal cancer, endometrial cancer, eye cancer, gallbladder cancer, gastric cancer, gestational trophoblastic tumors (GTT), hairy cell leukaemia, head and neck cancer, Hodgkin lymphoma, kidney cancer, laryngeal cancer, leukaemia, liver cancer, lung cancer, lymphoma, skin cancer, e.g., melanoma skin cancer, mesothelioma, men's cancer, molar pregnancy, mouth and oropharyngeal cancer, myeloma, nasal and sinus cancers, nasopharyngeal cancer, Non Hodgkin lymphoma (NHL), oesophageal cancer, ovarian cancer, pancreatic cancer, penile cancer, prostate cancer, rare cancers, rectal cancer, salivary gland cancer, secondary cancers, skin cancer (non melanoma), soft tissue sarcoma, stomach cancer, testicular cancer, thyroid cancer, unknown primary cancer, uterine cancer, vaginal cancer, or vulval cancer.

In some embodiments, the cancer has impaired synthesis of an amino acid, e.g., asparagine, phenylalanine, serine, methionine, or arginine, e.g., wherein synthesis of the amino acid is less than 50%, 40%, 30%, 20%, 10%, 5%, 2%, or 1% of the corresponding rate in a non-cancerous cell of the same tissue type from the same subject, or wherein the level of the amino acid in the tumor without treatment with a tumor starvation agent (e.g., an amino acid degradative enzyme) is less than 50%, 40%, 30%, 20%, 10%, 5%, 2%, or 1% of the corresponding level in a non-cancerous cell of the same tissue type from the same subject. In some embodiments, the cancer has a mutation in an amino acid synthesis gene.

In some embodiments, the method comprises administering a therapeutically effective amount of the genetically engineered, enucleated erythroid cells to the subject at least 2, 3, 4, 5, or 10 times.

In some embodiments, the method comprises administering a therapeutically effective amount of the genetically engineered, enucleated erythroid cells to the subject every 1, 2, or 3 months, or every 1-2 or 2-3 months. In some embodiments, the method comprises administering 20-50 U of the asparaginase molecule every 1-3 months, e.g., administering 20-30, 30-40, or 40-50 U of the asparaginase molecule every 1 month, administering 20-30, 30-40, or 40-50 U of the asparaginase every 2 months, or administering 20-30, 30-40, or 40-50 U of the asparaginase molecule every 3 months. In some embodiments, the method a dose comprises about 5×10⁹-1×10¹⁰, 1×10¹⁰-2×10¹⁰, 2×10¹⁰-5×10¹⁰, or 5×10¹⁰-1×10¹¹ cells.

In some embodiments, e.g., those involving reducing the concentration of an amino acid in a subject, the plasma concentration of the amino acid (e.g., asparagine or glutamine) is reduced by at least 10%, 20%, 30%, 40%, 50%, 60%, 70%, 80%, or 90% compared to before treatment (e.g., when measured 4 days after administration). In some embodiments, the concentration of the amino acid (e.g., asparagine or glutamine) is reduced by at least 10%, 20%, 30%, 40%, 50%, 60%, 70%, 80%, or 90% in the tumor compared to before treatment (e.g., when measured 4 days after administration). In some embodiments, the intracellular concentration of the amino acid (e.g., asparagine or glutamine) in a cancer cell is reduced by at least 10%, 20%, 30%, 40%, 50%, 60%, 70%, 80%, or 90% compared to before treatment (e.g., when measured 4 days after administration). In some embodiments, the extracellular concentration of the amino acid (e.g., asparagine or glutamine) in a tumor is reduced by at least 10%, 20%, 30%, 40%, 50%, 60%, 70%, 80%, or 90% compared to before treatment (e.g., when measured 4 days after administration). In some embodiments, the plasma concentration of the amino acid (e.g., asparagine or glutamine) is below 60, 50, 40, 30, 20, or 10 μM (e.g., when measured 4 days after administration). In some embodiments, the concentration of the amino acid (e.g., asparagine or glutamine) in the tumor is below 60, 50, 40, 30, 20, or 10 μM (e.g., when measured 4 days after administration).

In some embodiments, the method comprises administering the erythroid cells such that at least a subset of the erythroid cells reaches the bone marrow of the subject.

The disclosure contemplates all combinations of any one or more of the foregoing aspects and/or embodiments, as well as combinations with any one or more of the embodiments set forth in the detailed description and examples.

Although methods and materials similar or equivalent to those described herein can be used in the practice or testing of the present invention, suitable methods and materials are described below. All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Oct. 24, 2017. When one gene or protein references a plurality of sequence accession numbers, all of the sequence variants are encompassed.

### BRIEF DESCRIPTION OF THE DRAWINGS

FIG. 1 is a flow cytometry plot showing erythroid cells expressing an asparaginase molecule fused to transmembrane domain of Kell.

FIG. 2 is a flow cytometry plot showing erythroid cells expressing anti-CD33 scFv fused to Glycophorin A anchor, and its ability to bind to CD33.

FIG. 3A is a flow cytometry plot showing CFSE-positive gated AML MV4-11 cells binding to anti-CD33 scFv-HA tag-Glycophorin A on erythroid cells. FIG. 3B is a flow cytometry plot showing CFSE-positive gated AML MV4-11 cells not binding to HA tag-Glycophorin A on erythroid cells.

FIG. 4 is a graph showing a time course of depletion of asparagine by engineered, erythroid cells expressing an asparaginase molecule fused to transmembrane domain of Kell (squares). Circles: Control erythroid cells not expressing an asparaginase molecule.

FIGS. 5A and 5B show pharmacokinetic and pharmacodynamic data for asparaginase-conjugated mRBCs in C57BL/6J mice. FIG. 5A shows pharmacokinetic data for asparaginase labeled cells tracked by Cy5. FIG. 5B shows pharmacodynamic data (asparagine plasma concentration) for the mice of FIG. 5A. Arrows indicate dosing of control or asparaginase-labeled mRBCs into mice. Solid line: group 1, control mRBCs. Dashed and dotted line: group 2, ˜0.052 asparaginase units/mouse. Dashed line: group 3, ˜0.022 asparaginase units/mouse. Dotted line: group 4, ˜0.0082 units/mouse.

FIG. 6A-6B show results of administering erythroid cells comprising an asparaginase molecule to mice. FIG. 6A shows the pharmacokinetic profile of mRBCs labeled with Erwinia chrysanthemi asparaginase. Dosing days are indicated by arrows. Groups 1-4 are described in Table 9. The x axis is time (from 0 to over 80 days). The y axis indicates the percent of dosed cells, as measured by Cy5 detection. FIG. 6B shows anti-asparaginase serum titers in C57BL/6J mice dosed with mRBCs labeled to various degrees with Erwinia chrysanthemi asparaginase. Dosing days are indicated with arrows. The x axis is time (from 0 to over 80 days). The y axis is the log₁₀ of antibody titer.

FIG. 7 is a graph illustrating the percentage of Cy5+ cells in blood samples relative to time in 5 groups of mice described in Table 10.

FIGS. 8A and 8B are graphs illustrating levels of plasma asparagine and glutamine, respectively, over time, in 5 groups of mice described in Table 10.

FIG. 9 is a graph illustrating body weight over time of the 5 groups of mice described in Table 10.

FIG. 10 is a graph illustrating the percentage of Cy5+ cells in blood samples relative to time in 5 groups of mice described in Table 11.

FIG. 11 is a graph illustrating the total number of MV-4-11 cancer cells in blood of 5 groups of mice described in Table 11, over time.

FIG. 12 is a graph illustrating the number of MV-4-11 cancer cells in 35 ul blood samples of 5 groups of mice described in Table 11, 4 days after dosing. ** indicates P≤0.01; **** indicates P≤0.0001. The reported P values were determined using two-tailed unpaired t test.

FIG. 13 is a graph illustrating the fraction of live cells remaining after treatment with PseuGLNase (y axis), for 7 types of target cells (x axis). The target cells are erythroid precursors (control), MV4-11, MOLM-13, THP1, HL60, B16-F10, and RPMI 8226.

FIG. 14 is a FACS plot illustrating SSC-H levels on the y axis and BL2-H::PE-H levels on the x axis. 34% of cells had HA levels above the threshold.

### DETAILED DESCRIPTION OF THE INVENTION

### Definitions

“Acquire” or “acquiring” as the terms are used herein, refer to obtaining possession of a physical entity (e.g., a sample, a cell, a polypeptide, a nucleic acid, or a sequence), or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, or performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

As used herein, the term “antibody molecule” refers to a protein, e.g., an immunoglobulin chain or fragment thereof, comprising at least one immunoglobulin variable domain sequence. In some embodiments, the antibody molecule binds specifically to a target (e.g., CD33), such as a carbohydrate, polynucleotide, polypeptide, etc., through at least one antigen recognition site, located in the variable region of the immunoglobulin molecule. The term “antibody molecule” encompasses antibodies, antibody fragments (such as Fab, Fab′, F(ab′)2, Fv), single chain (ScFv) and domain antibodies), and fusion proteins including an antibody portion, and any other modified configuration of an immunoglobulin molecule that includes an antigen recognition site.

A “variable region” of an antibody molecule refers to the variable region of the antibody molecule light chain or the variable region of the antibody molecule heavy chain, either alone or in combination. As known in the art, the variable regions of the heavy and light chain each consist of four framework regions (FR) connected by three complementarity determining regions (CDRs) that contain hypervariable regions. The CDRs in each chain are held together in close proximity by the FRs and, with the CDRs from the other chain, contribute to the formation of the antigen-binding site of antibodies. The positions of the CDRs and FRs may be determined using various well-known methods, e.g., Kabat, Chothia, the international ImMunoGeneTics database (IMGT) (on the worldwide web at imgt.org), and AbM (see, e.g., Johnson et al, Nucleic Acids Res., 29:205-206 (2001); Chothia and Lesk, J. Mol. Biol., 196:901-917 (1987); Chothia et al, Nature, 342:877-883 (1989); Chothia et al, J. Mol. Biol., 227:799-817 (1992); Al-Lazikani et al, J. Mol. Biol, 273:927-748 (1997)). In some embodiments, the CDRs of an antibody molecule are determined according to Kabat, Chothia, or a combination thereof.

In some embodiments, the antibody molecule is a monoclonal antibody molecule. As used herein, “monoclonal antibody molecule” or “monoclonal antibody” refers to an antibody molecule obtained from a population of substantially homogeneous antibody molecules, e.g., wherein individual antibodies including the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen.

As used herein, the term “asparaginase molecule” refers to a polypeptide having an activity of degrading L-asparagine, e.g., to aspartic acid and ammonia. The activity of degrading L-asparagine is also referred to herein as asparagine-degrading activity. In some embodiments, the asparaginase molecule has both asparagine-degrading activity and glutamine-degrading activity (i.e., glutaminase activity). “Glutamine-degrading activity”, as used herein, refers to the ability of an enzyme to catalyze the hydrolysis of glutamine to glutamate and ammonia. Thus, in some embodiments, the asparaginase molecule catalyzes the hydrolysis of asparagine and glutamine to aspartic acid and glutamic acid, respectively, and ammonia. In some embodiments, the asparaginase molecule lacks glutamine-degrading activity. Methods for assaying the asparagine-degrading or glutamine-degrading activity of asparaginase molecules are described for example, in Gervais and Foote (2014) Mol. Biotechnol. 45(10): 865-877, which is herein incorporated by reference in its entirety).

As used herein, a “cell surface marker” refers to a structure (e.g., a protein) that is in contact with a cell and at least partially at the surface of the cell, and can be detected to distinguish the cell from one or more other types of cell from the same individual. In some embodiments, the presence and/or absence of a cell surface marker can be used to distinguish the cell from other types of cells. In some embodiments, the quantity of cell surface marker present on the surface of a cell can be used to distinguish the cell from other types of cells. In some embodiments, the cell surface marker is a transmembrane protein or a lipid-anchored protein. In some embodiments, the cell surface marker can be used to distinguish a cancer cell from a non-cancerous cell. In some embodiments, the cell surface marker can be used to distinguish a cell of a first tissue type from a cell of a second tissue type.

As used herein, a “cell targeting moiety” refers to a polypeptide that can bind a target cell and can be used to distinguish the target cell from one or more other types of cell from the same individual. In some embodiments, the cell targeting moiety comprises an antibody molecule. In some embodiments, a cell targeting moiety specifically binds to a cell surface marker (e.g., a cell surface protein) present on or in a target cell. Exemplary cell targeting moieties include, but are not limited to an antibody molecule, a specific binding protein, a ligand (e.g., a receptor ligand on a target cell), or a receptor for a ligand on a target cell. For example, in some embodiments the cell surface marker is CD33 and the cell targeting moiety may comprise an anti-CD33 antibody molecule or a specific binding partner for CD33, e.g., a CD33-binding fragment or a CD33 ligand, e.g., a naturally-occurring CD33 ligand.

An “amino acid degradative enzyme,” as used herein, refers to an enzyme that reduces the local activity or concentration of an amino acid substrate, and which forms or breaks a covalent bond in the amino acid. In some embodiments, the amino acid degradative enzyme degrades, cleaves, or modifies (e.g., by the addition of a functional group) the amino acid. In some embodiments, the amino acid degradative enzyme hydrolyzes a bond in an amino acid.

“Derived from” as that term is used herein, indicates a relationship between a first and a second molecule. It generally refers to structural similarity between the first molecule and a second molecule and does not connote or include a process or source limitation on a first molecule that is derived from a second molecule.

As used herein, “enucleated” refers to a cell, e.g., a reticulocyte or mature red blood cell, that lacks a nucleus. In some embodiments an enucleated cell is a cell that has lost its nucleus through differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell, into a reticulocyte or mature red blood cell. In some embodiments, an enucleated cell is a cell that has lost its nucleus through in vitro differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell into a reticulocyte or mature red blood cell. In some embodiments, the enucleated cell is a platelet, a reticulocyte, or an erythrocyte.

“Erythroid cell” as used herein, includes a nucleated red blood cell, a red blood cell precursor, an enucleated mature red blood cell, and a reticulocyte. For example, any of a cord blood stem cell, a CD34+ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), a reticulocyte, an erythrocyte, an induced pluripotent stem cell (iPSC), a mesenchymal stem cell (MSC), a polychromatic normoblast, an orthochromatic normoblast, is an erythroid cell. A preparation of erythroid cells can include any of these cells or a combination thereof. In some embodiments, the erythroid cells are immortal or immortalized cells. For example, immortalized erythroblast cells can be generated by retroviral transduction of CD34+ hematopoietic progenitor cells to express Oct4, Sox2, Klf4, cMyc, and suppress TP53 (e.g., as described in Huang et al. (2014) Mol. Ther. 22(2): 451-463). In addition, the cells may be intended for autologous use or provide a source for allogeneic transfusion. In some embodiments, erythroid cells are cultured. In some embodiments, an erythroid cell is an enucleated red blood cell. In some embodiments, the erythroid cell is an erythrocyte or a reticulocyte. In some embodiments, the erythroid cells are isolated erythroid cells.

As used herein, the term “exogenous polypeptide” refers to a polypeptide that is not produced by a wild-type cell of that type or is present at a lower level in a wild-type cell than in a cell containing the exogenous polypeptide. In some embodiments, an exogenous polypeptide is a polypeptide encoded by a nucleic acid that was introduced into the cell, which nucleic acid is optionally not retained by the cell. In some embodiments, an exogenous polypeptide is a polypeptide conjugated to the surface of the cell by chemical or enzymatic means, for instance, using click chemistry or sortase-mediated conjugation.

“Genetically engineered,” as used herein in reference to cells, refers to a cell that comprises a nucleic acid sequence (e.g., DNA or RNA (e.g., mRNA)) that is not present in, or is present at a different level than, an otherwise similar cell under similar conditions that is not engineered (an exogenous nucleic acid), or a cell that comprises a polypeptide expressed from said nucleic acid. In some embodiments, a genetically engineered cell has been altered from its native state by the introduction of an exogenous nucleic acid, or is the progeny of such an altered cell. In some embodiments, a genetically engineered cell comprises an exogenous nucleic acid (e.g., DNA or RNA, e.g., mRNA). In some embodiments, a genetically engineered cell comprises an exogenous protein expressed from an exogenous nucleic acid, but does not comprise some or all of said exogenous nucleic acid. For instance, in some embodiments, the genetically engineered cell loses the exogenous nucleic acid during differentiation, e.g., due to enucleation or nucleic acid degradation. In some embodiments, the exogenous nucleic acid comprises a chromosomal or extra-chromosomal exogenous nucleic acid which is expressed as RNA, e.g., mRNA. In some embodiments, the exogenous nucleic acid sequence comprises a chromosomal or extra-chromosomal nucleic acid encodes a polypeptide and/or is expressed as a polypeptide. In some embodiments, the exogenous nucleic acid comprises a gene of interest (e.g., a gene encoding an amino acid degradative enzyme) operably linked to a promoter (e.g., an inducible promoter or a constitutive promoter).

“Operably linked” refers to the association of nucleic acid sequences on a single nucleic acid fragment so that the function of one is affected by the other. A regulatory element is operably linked with a coding sequence when it is capable of affecting the expression of the gene coding sequence, regardless of the distance between the regulatory element and the coding sequence.

As used herein, the term “expression” refers to the transcription and accumulation of RNA (e.g., sense (mRNA) or anti-sense RNA) from a nucleic acid, and/or to translation of an mRNA into a polypeptide.

As used herein, the terms “peptide,” “polypeptide,” and “protein” are used interchangeably to refer to any chain of two or more natural or unnatural amino acid residues, regardless of post-translational modification (e.g., glycosylation or phosphorylation), constituting all or part of a naturally-occurring or non-naturally occurring polypeptide.

As used herein, the term “variant” of a polypeptide refers to a polypeptide having at least one sequence difference compared to that polypeptide, e.g., one or more substitutions, insertions, or deletions. The variant may have one or more amino acid residue differences as compared to a reference polypeptide. In some embodiments, a variant has at least about 70%, 75%, 80%, 85%, 90%, 91%, 92%, 93%, 94%, 95%, 96%, 97%, 98%, or 99% identity to that polypeptide. A variant may include a fragment (e.g., an enzymatically active fragment of a polypeptide (e.g., an enzyme)). In some embodiments, a fragment may lack up to about 1, 2, 3, 4, 5, 10, 20, 30, 40, 50, or 100 amino acid residues on the N-terminus, C-terminus, or both ends (each independently) of a polypeptide, as compared to the full-length polypeptide. Variants may occur naturally or be non-naturally occurring. Non-naturally occurring variants may be generated using mutagenesis methods known in the art. Variant polypeptides may comprise conservative or non-conservative amino acid substitutions, deletions or additions.

As used herein, the term “percent (%) sequence identity” or “percent (%) identity,” refers to the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981.

### Exemplary Polypeptides and Uses Thereof

In some embodiments, one or more of the exogenous polypeptides (e.g., amino acid degradative enzyme or targeting moiety) is a fusion protein, e.g., a fusion with an endogenous red blood cell protein or fragment thereof, e.g., an intracellular protein or a transmembrane protein, e.g., GPA, Kell, CD71, or a transmembrane fragment thereof. In some embodiments, the transmembrane protein is a type-1 transmembrane protein, a type-2 transmembrane protein, or a type-3 transmembrane protein. In some embodiments, the transmembrane protein or fragment thereof has an extracellular N-terminus, and in other embodiments, the transmembrane protein or fragment thereof has an extracellular C-terminus. In some embodiments, one or more of the exogenous polypeptides is not a fusion protein. In some embodiments, one or more of the exogenous polypeptides is not fused to an endogenous erythrocyte protein or fragment thereof.

An exemplary polypeptide, e.g., a polypeptide selected from any of Tables 1-7A, includes:

a) a naturally occurring form of the polypeptide;

b) the polypeptide (e.g., an enzymatically active polypeptide) having a sequence appearing in a database, e.g., GenBank database, on Oct. 24, 2017;

c) a polypeptide (e.g., an enzymatically active polypeptide) having a sequence that differs by no more than 1, 2, 3, 4, 5 or 10 amino acid residues from a sequence of a) or b);

d) a polypeptide (e.g., an enzymatically active polypeptide) having a sequence that differs at no more than 1, 2, 3, 4, 5 or 10% its amino acids residues from a sequence of a) or b);

e) a polypeptide (e.g., an enzymatically active polypeptide) having a sequence that does not differ substantially from a sequence of a) or b); or

f) a polypeptide having a sequence of c), d), or e) that does not differ substantially in a biological activity, e.g., an enzymatic activity (e.g., specificity or turnover) or binding activity (e.g., binding specificity or affinity) from a polypeptide having the sequence of a) or b). Candidate peptides under f) can be made and screened for similar activity as described herein and would be equivalent hereunder if expressed in enucleated erythroid cells as described herein).

In some embodiments, a polypeptide comprises a polypeptide or fragment thereof, e.g., all or a fragment of a polypeptide of a), b), c), d), e), or f) of the preceding paragraph. In some embodiments, the polypeptide comprises a fusion polypeptide comprising all or a fragment of a polypeptide of a), b), c), d), e), or f) of the preceding paragraph and additional amino acid sequence. In some embodiments the additional amino acid sequence comprises all or a fragment of polypeptide of a), b), c), d), e), or f) of the preceding paragraph for a different polypeptide.

In some embodiments, an exogenous polypeptide described herein is at least 200, 300, 400, 500, 600, 700, or 800 amino acids in length. In some embodiments, the exogenous polypeptide is between 200-300, 300-400, 400-500, 500-600, 600-700, or 700-800 amino acids in length. In some embodiments, the exogenous polypeptide is less than 500, 450, 400, 350, or 300 amino acids in length. In some embodiments, the amino acid degradative enzyme is less than 400, 350, or 300 amino acids in length.

In some embodiments, a cell herein comprises at least 1,000, 2,500, 5,000, 10,000, 15,000, 20,000, 25,000, 30,000, 50,000, 100,000, 200,000, or 500,000 copies of an exogenous polypeptide described herein, e.g., an amino acid degradative enzyme. In some embodiments, the cell comprises at least 1,000, 2,500, 5,000, 10,000, 15,000, 20,000, 25,000, 30,000, 50,000, 100,000, 200,000, or 500,000 copies of the cell targeting moiety.

In some embodiments, the exogenous protein (e.g., amino acid degradative enzyme or targeting moiety) described herein comprises a leader sequence (e.g., a naturally-occurring leader sequence or a synthetic leader sequence). In some embodiments, the exogenous protein lacks a leader sequence (e.g., is genetically modified to remove a naturally-occurring leader sequence). In some embodiments, the exogenous protein comprises an N-terminal methionine residue. In some embodiments, the exogenous protein lacks an N-terminal methionine residue.

### Amino Acid Degradative Enzymes

Amino acid metabolism is fundamental to life, and enzymes that catalyze amino acid synthesis and breakdown are found in prokaryotes, eukaryotes, and archaea. A number of enzymes that degrade amino acids are useful in treating cancer. Cancer cells are often auxotrophic for one or more amino acids (e.g., due to mutations in the pathway that synthesizes that amino acid). As a result, the cancer cell's growth and viability depends on taking up the amino acid from their environment, making the cancer cells more sensitive to amino acid starvation than a subject's noncancerous cells (e.g., a noncancerous cell of the same tissue and/or type). Several amino acid degradative enzymes suitable for use in the treatment of cancer are described herein.

Without wishing to be bound by theory, an erythroid cell may be a particularly advantageous context for delivering an amino acid degradative enzymes for several reasons. First, some amino acid degradative enzymes have toxic effects when administered systemically (see, e.g., Hijiya et al., “Asparaginase-associated toxicity in children with acute lymphoblastic leukemia” Leuk Lymphoma. 2016; 57(4):748-57; Pieters et al., “L-asparaginase treatment in acute lymphoblastic leukemia: a focus on Erwinia asparaginase” Cancer. 2011 Jan. 15; 117(2): 238-249). This toxicity may be dramatically ameliorated by using an erythroid cell comprising the amino acid degradative enzyme, as shown, for instance, in Example 7 herein. Second, certain erythroid cells disclosed herein comprise a targeting moiety which can concentrate the erythroid cells, and therefore the amino acid degradative enzyme, in the environment of the tumor. This may further increase the specificity of the therapy. Third, without wishing to be bound by theory, erythroid cells can have different biodistribution from other therapeutics such as purified proteins, allowing the erythroid cells, and therefore the amino acid degradative enzyme, to reach anatomic sites (e.g., vascularized anatomic sites (e.g., the bone marrow)) not normally accessible to other therapeutics. For instance, in some embodiments, at least a subset of the erythroid cells reach the bone marrow in the subject, e.g., a subject having leukemia. This can be beneficial because it allows the erythroid cells to reach leukemic cells in the bone marrow which might be inaccessible to other therapeutics.

In some embodiments, the amino acid degradation enzyme comprises an asparaginase molecule, serine dehydratase molecule, serine hydroxymethyltransferase molecule, NAD-dependent L-serine dehydrogenase molecule, arginase molecule, arginine deiminase molecule, methionine gamma-lyase molecule, L-amino acid oxidase molecule, S-adenosylmethionine synthase molecule, cystathionine gamma-lyase molecule, indoleamine 2,3-dioxygenase molecule, or phenylalanine ammonia lyase molecule, e.g., as described herein. In some embodiments, the amino acid degradation enzyme comprises glutaminase, glutamine-pyruvate transaminase, branched-chain-amino-acid transaminase, amidase, arginine decarboxylase, aromatic-L-amino-acid decarboxylase, cysteine lyase, or argininosuccinate lyase, e.g., as described herein. In some embodiments, the amino acid degradative enzyme comprises an enzymatically active polypeptide having at least 70%, 80%, 85%, 90%, 95%, 96%, 97%, 98%, or 99% identity to the amino acid sequence of any of SEQ ID NOs: 116-129.

### Asparaginase Molecules

In some embodiments, the amino acid degradative enzyme comprises an asparaginase molecule or a fragment or variant thereof. For example, an exogenous asparaginase molecule can comprise a sequence of any of SEQ ID NOs: 3 to 8 or 68 to 70, or an asparagine-degrading fragment thereof, or a sequence with at least 70%, 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In some embodiments, the asparaginase molecule is an asparaginase molecule described in Covini et al., “Expanding Targets for a Metabolic Therapy of Cancer: L-asparaginase”, Recent Pat Anticancer Drug Discov. 2012 January; 7(1):4-13, which is herein incorporated by reference in its entirety, including Table 1 therein, or a sequence with at least 70%, 80%, 85%, 90%, 95%, 96%, 97%, 98%, or 99% identity thereto. In some embodiments, the asparaginase molecule is an asparaginase molecule from Arabidopsis thaliana (e.g., having a Kₘ of 4 mM or less for asparagine, a k_{cat} of 0.23 s⁻¹ or greater for asparagine, or a combination thereof), Homo sapiens (e.g., having a Kₘ of 0.656 mM or less for asparagine, a k_{cat} of 1.09 s⁻¹ or greater for asparagine, or a combination thereof), or Helicobacter pylori (e.g., having a Kₘ of 0.290 mM or less for asparagine, a k_{cat} of 19.2 s⁻¹ or greater for asparagine, a Kₘ of 46.4 mM or less for glutamine, a k_{cat} of 22.1 s⁻¹ or greater for glutamine or a combination thereof). In some embodiments, the asparaginase molecule has at least one activity characteristic of an asparaginase molecule of SEQ ID NOs: 3 to 8 or 68 to 70, e.g., it can metabolize asparagine, e.g., with a k_{cat} at least 90%, 80%, 70%, 60%, or 50% of that of an asparaginase molecule of any one of SEQ ID NOs: 3 to 8 or 68 to 70 or a Km less than 150%, 125%, 100%, 75%, or 50% of the Kₘ of an asparaginase molecule of any one of SEQ ID NOs: 3 to 8, or a combination thereof. Asparagine metabolism can be measured, e.g., using an assay of Gervais and Foote, “Recombinant deamidated mutants of Erwinia chrysanthemi L-asparaginase have similar or increased activity compared to wild-type enzyme.” Mol Biotechnol. 2014; 45(10): 865-877, which is herein incorporated by reference in its entirety. Functional asparaginase polypeptides are described, e.g., in Gervais and Foote, (supra), Nguyen et al. “Design and Characterization of Erwinia Chrysanthemi L-Asparaginase Variants with Diminished L-Glutaminase Activity.” J Biol Chem. 2016; 291(34): 17664-17676, and Moola et al. “Erwinia chrysanthemi L-asparaginase: epitope mapping and production of antigenically modified enzymes.” Biochemical Journal. 1994; 302(3): 921-927., each of which is herein incorporated by reference in its entirety. In some embodiments, the asparaginase polypeptide comprises Erwinia chrysanthemi asparaginase (SEQ ID NO: 3) or a fragment or variant thereof.

Numerous asparaginase molecules have been identified in bacteria, plants, yeast, algae, fungi and mammals, and may be used as described herein. For example, asparaginase molecules may be obtained from a variety of species including, but not limited to, Escherichia coli (see, e.g., UnitProt Accession No. P00805), Erwinia carotovora (also known as Pectobacterium atrosepticum; see, e.g., GenBank Accession No. AAS67027), Erwinia chrysanthemi (also known as Dickeya chrysanthemi; see, e.g., UniProt Accession Nos. P06608, and AAS67028; and GenBank Accession No. CAA31239); Erwinia carotovora (also known as Pectobacterium atrosepticum; see, e.g., GenBank Accession Nos. AAS67027, AAP92666 and Q6Q4F4), Pseudomonas stutzeri (see, e.g., GenBank Accession No. AVX11435), Delftia acidovoras (also known as Pseudomonas acidovorans; see, e.g., GenBank Accession No. ABX36200), Pectobacterium carotovorum (also known as Erwinia aroideae; see, e.g., NCBI Reference No. WP_015842013), Thermus thermophilus (see, e.g., GenBank Accession Nos. BAD69890 and BAW01549), Thermus aquaticus (see, e.g., GenBank Accession Nos. KOX89292 and EED09821), Staphylococcus aureus (see, e.g., GenBank Accession Nos KII20890, ARI73732, and PJJ95560), Wolinella succinogenes (also known as Vibrio succinogenes; see, e.g., GenBank Accession No. CAA61503), Citrobacter freundi (see, e.g., GenBank Accession No. EXF30424), Proteus vulgaris (see, e.g., GenBank Accession No. KGA60073), Zymomonas mobilis (see, e.g., GenBank Accession Nos. AHB10760, ART93886, AAV90307, AEH63277, and ACV76074), Bacillus subtilis (see, e.g., UniProt Accession No. 03448), Bacillus licheniformis (see, e.g., GenBank Accession Nos. ARW56273, ARW54537, ARW44915, and AOP17372), Bacillus circulans (see, e.g., GenBank Accession Nos. KLV25750, PAE13094, PAD89980, PAD81349, PAD90008, and PAE13121), Enterobacter aerogenes (see, e.g., NCBI Reference No. YP_004594521, and GenBank Accession No. SFX86538), Serratia marcescens (see, e.g., GenBank Accession Nos. ALD46588, ALE95248, OSX81952, and PHI53192), Wolinella succinogenes (see, e.g., UniProt Accession No. P50286), Helicobacter pylori (see, e.g., UniProt Accession No. 025424), and Cavia porcellus (guinea pig) (see, e.g., UniProt Accession No. HOW0T5), Aspergillus nomius (see, e.g., NCBI Reference No. XP_015407819), Aspergillus terreus (see, e.g., GenBank Accession Nos. EAU36905 and KT728852), Aspergillus fischeri (NCBI Reference No. XP_001265372), Aspergillus fumigatus (NCBI Reference No. XP_750028), Glarea lozoyensis (see, e.g., NCBI Reference No. XP_008086736), Saccharomyces cerevisae (see, e.g., NCBI Reference No. NP 010607), Cyberlindnera jadinii (also known as Candida utilis; see, e.g., GenBank Accession No. CEP24033); Meyerozyma guilliermondii (also known as Candida guilliermondii; see, e.g., NCBI Reference No. XP_001485067; and GenBank Accession No. EDK36913), and Rhodotorula toruloides (see, e.g., NCBI Reference Nos. XP_016274149.1 and XP_016272508). Any of the above-identified asparaginase molecules (or functional variants thereof) may be used as described herein.

In some embodiments, the asparaginase molecule has a leader sequence (e.g., a naturally-occurring leader sequence or a synthetic leader sequence). In some embodiments, the asparaginase molecule lacks a leader sequence (e.g., is genetically modified to remove a naturally-occurring leader sequence). In some embodiments, the asparaginase molecule has an N-terminal methionine residue. In some embodiments, the asparaginase molecule lacks an N-terminal methionine residue.

Asparaginase can be used, e.g., in the treatment of a leukemia (e.g., acute myeloid leukemia (AML), acute lymphoblastic leukemia (ALL), lymphoblastic lymphoma), a lymphoma (e.g., NK/T Cell lymphoma or non-Hodgkin lymphoma), pancreatic cancer, ovarian cancer, fallopian cancer, and peritoneal cancer.

In some embodiments, an erythroid cell described herein is contacted with, comprises, or expresses a nucleic acid (e.g., DNA or RNA) encoding an asparaginase molecule described herein. In some embodiments, a genetically engineered enucleated erythroid cell comprises an asparaginase molecule described herein.

Serine Dehydratase Molecules

In some embodiments, the amino acid degradative enzyme comprises serine dehydratase or a fragment or variant thereof. In some embodiments, a serine dehydratase molecule is polypeptide having an amino acid sequence at least 70%, 80%, 85%, 90%, 95%, 96%, 97%, 98%, or 99% identical to the amino acid sequence of the serine dehydratase of SEQ ID NO: 9, 71, or 72 or to a serine-degrading fragment thereof, and having an activity of degrading L-serine by deamination, to produce pyruvate and ammonia. In some embodiments, serine dehydratase activity is measured in an assay according to Sun et al, “Crystal structure of the pyridoxal-5′-phosphate-dependent serine dehydratase from human liver.” Protein Sci. 2005 March; 14(3):791-8. Epub 2005 Feb. 2, which is herein incorporated by reference in its entirety. In some embodiments, the serine dehydratase molecule has a k_{cat} at least 90%, 80%, 70%, 60%, or 50% of that of a serine dehydratase of SEQ ID NO: 9, 71, or 72 or a Kₘ less than 150%, 125%, 100%, 75%, or 50% of the Kₘ of a serine dehydratase of SEQ ID NO: 9, 71, or 72, e.g., in an assay according to Sun et al., supra. In some embodiments, the serine dehydratase molecule uses a pyridoxal phosphate (PLP) coenzyme. In some embodiments, the serine dehydratase molecule is derived from an enzyme from a prokaryote or a eukaryote (e.g., a fungus such as yeast, or an animal, e.g., mammal, e.g., human).

Serine starvation leads to reduced viability in some cancers, e.g., p53 null cancers (see, Maddocks et al. “Serine starvation induces stress and p53-dependent metabolic remodeling in cancer cells” Nature 493, 542-546 (24 Jan. 2013), which is herein incorporated by reference in its entirety.

In some embodiments, an erythroid cell described herein is contacted with, comprises, or expresses a nucleic acid (e.g., DNA or RNA) encoding a serine dehydratase polypeptide described herein. In some embodiments, a genetically engineered enucleated erythroid cell comprises a serine dehydratase polypeptide described herein.

Serine Hydroxymethyltransferase Molecules

In some embodiments, the amino acid degradative enzyme comprises serine hydroxymethyltransferase or a fragment or variant thereof. In some embodiments, a serine hydroxymethyltransferase molecule is polypeptide having an amino acid sequence at least 70%, 80%, 85%, 90%, 95%, 96%, 97%, 98%, or 99% identical to the amino acid sequence of the serine hydroxymethyltransferase of SEQ ID NO: 10, 73, or 74 or to a serine-degrading fragment thereof, and having an activity of degrading L-serine, e.g., by cleaving the side chain from the backbone to produce glycine and formaldehyde. In some embodiments, serine hydroxymethyltransferase activity is measured in an assay according to Kruschwitz et al, “Expression, purification, and characterization of human cytosolic serine hydroxymethyltransferase.” Protein Expr Purif. 1995 August; 6(4):411-6, which is herein incorporated by reference in its entirety. In some embodiments, the serine hydroxymethyltransferase molecule has a k_{cat} at least 90%, 80%, 70%, 60%, or 50% of that of a serine hydroxymethyltransferase of SEQ ID NO: 10, 73, or 74 or a Kₘ less than 150%, 125%, 100%, 75%, or 50% of the Kₘ of a serine hydroxymethyltransferase of SEQ ID NO: 10, 73, or 74, e.g., in an assay according to Kruschwitz et al. supra. In some embodiments, the serine hydroxymethyltransferase molecule uses a pyridoxal phosphate (PLP) coenzyme. In some embodiments, the serine hydroxymethyltransferase molecule is derived from a prokaryotic or a eukaryotic enzyme.

Serine starvation leads to reduced viability in some cancers, e.g., p53 null cancers (see, Maddocks et al. “Serine starvation induces stress and p53-dependent metabolic remodelling in cancer cells” Nature 493, 542-546 (24 Jan. 2013), which is herein incorporated by reference in its entirety.

In some embodiments, an erythroid cell described herein is contacted with, comprises, or expresses a nucleic acid (e.g., DNA or RNA) encoding a serine hydroxymethyltransferase polypeptide described herein. In some embodiments, a genetically engineered enucleated erythroid cell comprises a serine hydroxymethyltransferase polypeptide described herein.

NAD-Dependent L-Serine Dehydrogenase Molecules

In some embodiments, the amino acid degradative enzyme comprises NAD-dependent L-serine dehydrogenase or a fragment or variant thereof. In some embodiments, a NAD-dependent L-serine dehydrogenase molecule is polypeptide having an amino acid sequence at least 70%, 80%, 85%, 90%, 95%, 96%, 97%, 98%, or 99% identical to the amino acid sequence of the NAD-dependent L-serine dehydrogenase of SEQ ID NO: 17, 88, or 89 or to an L-serine-degrading fragment thereof, and having an activity of oxidizing serine to produce 2-aminoacetaldehyde, carbon dioxide, and NADH. In some embodiments, NAD-dependent L-serine dehydrogenase activity is measured in an assay according to Tchigvintsev et al, “Biochemical and structural studies of uncharacterized protein PA0743 from Pseudomonas aeruginosa revealed NAD+-dependent L-serine dehydrogenase.” J. Biol. Chem. 287:1874-1883(2012), which is herein incorporated by reference in its entirety. In some embodiments, NAD-dependent L-serine dehydrogenase molecule has a k_{cat} at least 90%, 80%, 70%, 60%, or 50% of that of an L-amino acid oxidase of SEQ ID NO: 17, 88, or 89 or a Kₘ less than 150%, 125%, 100%, 75%, or 50% of the Kₘ of an L-serine dehydrogenase of SEQ ID NO: 17, 88, or 89, e.g., in an assay according to Tchigvintsev et al. supra.

Serine starvation leads to reduced viability in some cancers, e.g., as described herein.

In some embodiments, an erythroid cell described herein is contacted with, comprises, or expresses a nucleic acid (e.g., DNA or RNA) encoding a NAD-dependent L-serine dehydrogenase polypeptide described herein. In some embodiments, a genetically engineered enucleated erythroid cell comprises a NAD-dependent L-serine dehydrogenase polypeptide described herein.

Arginase Molecules

In some embodiments, the amino acid degradative enzyme comprises arginase or a fragment or variant thereof. In some embodiments, an arginase molecule is polypeptide having an amino acid sequence at least 70%, 80%, 85%, 90%, 95%, 96%, 97%, 98%, or 99% identical to the amino acid sequence of the arginase of SEQ ID NO: 11, 75, 76, or 77 or to an arginine-degrading fragment thereof, and having an activity of hydrolyzing arginine to ornithine and urea. In some embodiments, arginase activity is measured in an assay according to Berüter et al, “Purification and properties of arginase from human liver and erythrocytes.” Biochem J. 1978 Nov. 1; 175(2):449-54, which is herein incorporated by reference in its entirety. In some embodiments, the arginase molecule has a k_{cat} at least 90%, 80%, 70%, 60%, or 50% of that of an arginase of SEQ ID NO: 11, 75, 76, or 77 or a Kₘ less than 150%, 125%, 100%, 75%, or 50% of the Kₘ of an arginase of SEQ ID NO: 11, 75, 76, or 77 e.g., in an assay according to Berüter et al. supra. In some embodiments, the arginase molecule uses a manganese ion cofactor. In some embodiments, the arginase molecule is derived from a prokaryotic, eukaryotic, or archaeal enzyme.

Arginase can be used, e.g., to reduce proliferation of arginine-auxotrophic cancers. In some embodiments, the arginine-auxotrophic cancer is an epithelial cancer (see Vynnytska-Myronovska et al., “Single amino acid arginine starvation efficiently sensitizes cancer cells to canavanine treatment and irradiation.” Int J Cancer. 2012 May 1; 130(9):2164-75. doi: 10.1002/ijc.26221.

In some embodiments, an erythroid cell described herein is contacted with, comprises, or expresses a nucleic acid (e.g., DNA or RNA) encoding an arginase polypeptide described herein. In some embodiments, a genetically engineered enucleated erythroid cell comprises an arginase polypeptide described herein.

Arginine Deiminase Molecules

In some embodiments, the amino acid degradative enzyme comprises arginine deiminase or a fragment or variant thereof. In some embodiments, an arginine deiminase molecule is polypeptide having an amino acid sequence at least 70%, 80%, 85%, 90%, 95%, 96%, 97%, 98%, or 99% identical to the amino acid sequence of the arginine deiminase of SEQ ID NO: 12, 78, or 79 or to an arginine-degrading fragment thereof, and having an activity of hydrolyzing arginine to produce citrulline and ammonia. In some embodiments, arginine deiminase activity is measured in an assay according to El-Sayed et al, “Purification, immobilization, and biochemical characterization of 1-arginine deiminase from thermophilic Aspergillus fumigatus KJ434941: anticancer activity in vitro.” Biotechnol Prog. 2015 March-April; 31(2):396-405, which is herein incorporated by reference in its entirety. In some embodiments, the arginine deiminase molecule has a k_{cat} at least 90%, 80%, 70%, 60%, or 50% of that of an arginine deiminase of SEQ ID NO: 12, 78, or 79 or a Kₘ less than 150%, 125%, 100%, 75%, or 50% of the Kₘ of an arginine deiminase of SEQ ID NO: 12, 78, or 79, e.g., in an assay according to El-Sayed et al. supra.

Many solid tumors (e.g., breast tumors) have impaired arginine synthesis, e.g., due to a mutation in an arginine synthesis gene such as Argininosuccinate synthetase 1, and are dependent on uptake of arginine from their environment. See, e.g., Qui et al., “Arginine starvation impairs mitochondrial respiratory function in ASS1-deficient breast cancer cells” Sci Signal. 2014 Apr. 1; 7(319):ra31.

In some embodiments, an erythroid cell described herein is contacted with, comprises, or expresses a nucleic acid (e.g., DNA or RNA) encoding an arginine deiminase polypeptide described herein. In some embodiments, a genetically engineered enucleated erythroid cell comprises an arginine deiminase polypeptide described herein.

Methionine Gamma-Lyase Molecules

In some embodiments, the amino acid degradative enzyme comprises methionine gamma-lyase or a fragment or variant thereof. In some embodiments, a methionine gamma-lyase molecule is polypeptide having an amino acid sequence at least 70%, 80%, 85%, 90%, 95%, 96%, 97%, 98%, or 99% identical to the amino acid sequence of the L-methionine gamma-lyase of SEQ ID NO: 13, 80, or 81 or to a methionine-degrading fragment thereof, and having an activity of hydrolyzing L-methionine to methanethiol, ammonia, and 2-oxobutanoate. In some embodiments, methionine gamma lyase activity is measured in an assay according to Nakayama et al, “Purification of bacterial L-methionine gamma-lyase.” Anal. Biochem. 138:421-424(1984), which is herein incorporated by reference in its entirety. In some embodiments, the methionine gamma-lyase molecule has a k_{cat} at least 90%, 80%, 70%, 60%, or 50% of that of a methionine gamma-lyase of SEQ ID NO: 13, 80, or 81 or a Kₘ less than 150%, 125%, 100%, 75%, or 50% of the Kₘ of a methionine gamma-lyase of SEQ ID NO: 13, 80, or 81, e.g., in an assay according to Nakayama et al. supra. In some embodiments, the methionine gamma-lyase molecule uses a pyridoxal phosphate (PLP) coenzyme. In some embodiments, the methionine gamma-lyase degrades cysteine, serine, or homoserine. In some embodiments, the methionine gamma-lyase molecule is derived from a prokaryotic or eukaryotic (e.g., protozoan or plant) enzyme.

Tumors sensitive to methionine starvation include blastomas, e.g., glioblastomas, medulloblastomas, and neuroblastomas.

In some embodiments, an erythroid cell described herein is contacted with, comprises, or expresses a nucleic acid (e.g., DNA or RNA) encoding a methionine gamma-lyase polypeptide described herein. In some embodiments, a genetically engineered enucleated erythroid cell comprises a methionine gamma-lyase polypeptide described herein.

L-Amino Acid Oxidase Molecules

In some embodiments, the amino acid degradative enzyme comprises L-amino acid oxidase or a fragment or variant thereof. In some embodiments, an L-amino acid oxidase molecule is polypeptide having an amino acid sequence at least 70%, 80%, 85%, 90%, 95%, 96%, 97%, 98%, or 99% identical to the amino acid sequence of the L-amino acid oxidase of SEQ ID NO: 14, 82, or 83 or to an L-amino acid-degrading fragment thereof, and having an activity of oxidizing an L-amino acid, to produce a 2-oxo acid, ammonia, and hydrogen peroxide. In some embodiments, L-amino acid oxidase activity is measured in an assay according to Lazo et al, “Biochemical, biological and molecular characterization of an L-Amino acid oxidase (LAAO) purified from Bothrops pictus Peruvian snake venom.” Toxicon. 2017 Oct. 9; 139:74-86, which is herein incorporated by reference in its entirety. In some embodiments, the L-amino acid oxidase molecule has a k_{cat} at least 90%, 80%, 70%, 60%, or 50% of that of an L-amino acid oxidase of SEQ ID NO: 14, 82, or 83 or a Kₘ less than 150%, 125%, 100%, 75%, or 50% of the Kₘ of an L-amino acid oxidase of SEQ ID NO: 14, 82, or 83, e.g., in an assay according to Lazo et al. supra. In some embodiments, the L-amino acid oxidase is derived from an enzyme from a prokaryote, a eukaryote, e.g., a fungus, a mammal (e.g., human) or a reptile (e.g., snake, e.g., venomous snake).

Cancers sensitive to L-amino acid oxidase starvation include, e.g., cervical cancer.

In some embodiments, an erythroid cell described herein is contacted with, comprises, or expresses a nucleic acid (e.g., DNA or RNA) encoding an L-amino acid oxidase polypeptide described herein. In some embodiments, a genetically engineered enucleated erythroid cell comprises an L-amino acid oxidase polypeptide described herein.

S-Adenosylmethionine Synthase Molecules

In some embodiments, the amino acid degradative enzyme comprises S-adenosylmethionine synthase or a fragment or variant thereof. In some embodiments, a S-adenosylmethionine synthase molecule is polypeptide having an amino acid sequence at least 70%, 80%, 85%, 90%, 95%, 96%, 97%, 98%, or 99% identical to the amino acid sequence of the a S-adenosylmethionine synthase of SEQ ID NO: 15, 84, or 85 or to methionine-metabolizing fragment thereof, and having an activity of reacting methionine with ATP, to produce S-adenosylmethionine. In some embodiments, S-adenosylmethionine synthase activity is measured in an assay according to Markham et al, “S-Adenosylmethionine synthetase from Escherichia coli.” J. Biol. Chem. 255:9082-9092(1980), which is herein incorporated by reference in its entirety. In some embodiments, the L-amino acid oxidase molecule has a k_{cat} at least 90%, 80%, 70%, 60%, or 50% of that of a S-adenosylmethionine synthase of SEQ ID NO: 15, 84, or 85 or a Kₘ less than 150%, 125%, 100%, 75%, or 50% of the Kₘ of a S-adenosylmethionine synthase of SEQ ID NO: 15, 84, or 85 e.g., in an assay according to Markham et al. supra. In some embodiments, the S-adenosylmethionine synthase is derived from an enzyme from a prokaryote, a eukaryote, e.g., a fungus or a mammal.

Tumors sensitive to methionine starvation include blastomas, e.g., glioblastomas, medulloblastomas, and neuroblastomas.

In some embodiments, an erythroid cell described herein is contacted with, comprises, or expresses a nucleic acid (e.g., DNA or RNA) encoding an S-adenosylmethionine synthase polypeptide described herein. In some embodiments, a genetically engineered enucleated erythroid cell comprises an S-adenosylmethionine synthase polypeptide described herein.

Cystathionine Gamma-Lyase Molecules

In some embodiments, the amino acid degradative enzyme comprises cystathionine gamma-lyase or a fragment or variant thereof. In some embodiments, a cystathionine gamma-lyase molecule is polypeptide having an amino acid sequence at least 70%, 80%, 85%, 90%, 95%, 96%, 97%, 98%, or 99% identical to the amino acid sequence of the cystathionine gamma-lyase of SEQ ID NO: 16, 86, or 87 or to a cysteine-degrading fragment thereof, and having an activity of degrading methionine, e.g., to α-ketobutyrate. In some embodiments, cystathionine gamma-lyase activity is measured in an assay according to Zhu et al, “Kinetic properties of polymorphic variants and pathogenic mutants in human cystathionine gamma-lyase.” Biochemistry 47:6226-6232(2008), which is herein incorporated by reference in its entirety. In some embodiments, the cystathionine gamma-lyase oxidase molecule has a k_{cat} at least 90%, 80%, 70%, 60%, or 50% of that of a cystathionine gamma-lyase of SEQ ID NO: 16, 86, or 87 or a Kₘ less than 150%, 125%, 100%, 75%, or 50% of the Kₘ of a cystathionine gamma-lyase of SEQ ID NO: 16, 86, or 87, e.g., in an assay according to Zhu et al. supra. In some embodiments, the cystathionine gamma-lyase is derived from an enzyme from a prokaryote or a eukaryote, e.g., a mammal (e.g., human). In some embodiments, the cystathionine gamma-lyase molecule degrades methionine, e.g., see Stone et al., “De novo engineering of a human cystathionine-γ-lyase for systemic (L)-Methionine depletion cancer therapy.” ACS Chem Biol. 2012 Nov. 16; 7(11):1822-9. In some embodiments, the cystathionine gamma-lyase degrades cystathionine, cystine, cysteine, or L-homoserine.

In some embodiments, the cystathionine gamma-lyase is used to treat a blastoma, e.g., glioblastoma, medulloblastoma, or neuroblastoma.

In some embodiments, an erythroid cell described herein is contacted with, comprises, or expresses a nucleic acid (e.g., DNA or RNA) encoding a cystathionine gamma-lyase polypeptide described herein. In some embodiments, a genetically engineered enucleated erythroid cell comprises a cystathionine gamma-lyase polypeptide described herein.

Indoleamine 2,3-Dioxygenase Molecules

In some embodiments, the amino acid degradative enzyme comprises indoleamine 2,3-dioxygenase or a fragment or variant thereof. In some embodiments, an indoleamine 2,3-dioxygenase molecule is a heme-containing polypeptide having an amino acid sequence at least 70%, 80%, 85%, 90%, 95%, 96%, 97%, 98%, or 99% identical to the amino acid sequence of the indoleamine 2,3-dioxygenase 1 of SEQ ID NO: 18, 90, or 91 or to an tryptophan-degrading fragment thereof, and having an activity of oxidizing tryptophan, to produce N-formyl-L-kynurenine. In some embodiments, indoleamine 2,3-dioxygenase activity is measured in an assay according to Metz et al, “Novel tryptophan catabolic enzyme IDO2 is the preferred biochemical target of the antitumor indoleamine 2,3-dioxygenase inhibitory compound D-1-methyl-tryptophan.” Cancer Res. 67:7082-7087(2007), which is herein incorporated by reference in its entirety. In some embodiments, the L indoleamine 2,3-dioxygenase molecule has a k_{cat} at least 90%, 80%, 70%, 60%, or 50% of that of a indoleamine 2,3-dioxygenase 1 of SEQ ID NO: 18, 90, or 91 or a Kₘ less than 150%, 125%, 100%, 75%, or 50% of the Kₘ of a indoleamine 2,3-dioxygenase 1 of SEQ ID NO: 18, 90, or 91, e.g., in an assay according to Metz et al. supra. In some embodiments, the indoleamine 2,3-dioxygenase is derived from an enzyme from a prokaryote, a eukaryote, e.g., a fungus or a mammal (e.g., human).

In some embodiments, an erythroid cell described herein is contacted with, comprises, or expresses a nucleic acid (e.g., DNA or RNA) encoding an indoleamine 2,3-dioxygenase polypeptide described herein. In some embodiments, a genetically engineered enucleated erythroid cell comprises an indoleamine 2,3-dioxygenase polypeptide described herein.

Phenylalanine Ammonia Lyase Molecules

In some embodiments, the amino acid degradative enzyme comprises phenylalanine ammonia lyase or a fragment or variant thereof. In some embodiments, a phenylalanine ammonia lyase molecule is polypeptide having an amino acid sequence at least 70%, 80%, 85%, 90%, 95%, 96%, 97%, 98%, or 99% identical to the amino acid sequence of the phenylalanine ammonia lyase of SEQ ID NO: 19, 92, or 93 or to an phenylalanine-degrading fragment thereof, and having an activity of degrading phenylalanine to produce trans-cinnamate and ammonia. In some embodiments, phenylalanine ammonia lyase activity is measured in an assay according to Moffitt et al, “Discovery of two cyanobacterial phenylalanine ammonia lyases: kinetic and structural characterization.” Biochemistry 46:1004-1012(2007), which is herein incorporated by reference in its entirety. In some embodiments, the phenylalanine ammonia lyase molecule has a k_{cat} at least 90%, 80%, 70%, 60%, or 50% of that of a phenylalanine ammonia lyase of SEQ ID NO: 19, 92, or 93 or a Kₘ less than 150%, 125%, 100%, 75%, or 50% of the Kₘ of a phenylalanine ammonia lyase of SEQ ID NO: 19, 92, or 93, e.g., in an assay according to Moffitt et al. supra.

In some embodiments, an erythroid cell described herein is contacted with, comprises, or expresses a nucleic acid (e.g., DNA or RNA) encoding a phenylalanine ammonia lyase polypeptide described herein. In some embodiments, a genetically engineered enucleated erythroid cell comprises a phenylalanine ammonia lyase polypeptide described herein.

Glutaminase Molecules

In some embodiments, the amino acid degradative enzyme comprises a glutaminase or a fragment or variant thereof. In some embodiments, a glutaminase is polypeptide having an amino acid sequence at least 70%, 80%, 85%, 90%, 95%, 96%, 97%, 98%, or 99% identical to the amino acid sequence of the glutaminase of any of SEQ ID NO: 105-113 or to a glutamine-degrading fragment thereof, and having an activity of degrading glutamine to produce glutamate and ammonia. In some embodiments, the glutaminase has a k_{cat} at least 90%, 80%, 70%, 60%, or 50% of that of a glutaminase of any of SEQ ID NO: 105-113 or a Kₘ less than 150%, 125%, 100%, 75%, or 50% of the Kₘ of a glutaminase of any of SEQ ID NO: 105-113. In some embodiments, the glutaminase also has asparaginase activity. An enzyme may be both a glutaminase and an asparaginase.

In some embodiments, an erythroid cell described herein is contacted with, comprises, or expresses a nucleic acid (e.g., DNA or RNA) encoding a glutaminase molecule described herein. In some embodiments, a genetically engineered enucleated erythroid cell comprises a glutaminase molecule described herein.

### Linkers

Certain exogenous polypeptides described herein may comprise a linker, and certain exogenous nucleic acids described herein may encode a linker. In some embodiments, a linker comprises one or more amino acids that link two different polypeptide domains. In some embodiments, a linker is sufficiently flexible to allow the two linked domains to fold properly and/or have a biological activity, e.g., an enzymatic activity or a binding activity.

In some embodiments, the linker is disposed between a domain having amino acid degradative enzyme activity and a transmembrane domain. In some embodiments, the linker is disposed between a domain having targeting activity and a transmembrane domain. In some embodiments, the linker is disposed between a VH region and a VL region.

In some embodiments, the linker is a poly-glycine poly-serine linker. For example, in some embodiments, the linker comprises the amino acid sequence (Gly4Ser)ₙ, wherein (n=1-20). In some embodiments, the linker comprises or consists of a poly-glycine poly-serine linker with one or more amino acid substitutions, deletions, and/or additions and which lacks the amino acid sequence GSG. In some embodiments, a linker comprises or consists of the amino acid sequence (GGGXX)ₙGGGGS (SEQ ID NO: 62) or GGGGS(XGGGS)ₙ (SEQ ID NO: 63), where n is greater than or equal to one. In some embodiments, n is between 1 and 20, inclusive (e.g., n may be 1, 2, 3, 4, 5, 6, 7, 8, 9, 10, 11, 12, 13, 14, 15, 16, 17, 18, 19, or 20). Exemplary linkers include, but are not limited to, GGGGSGGGGS (SEQ ID NO: 64), GSGSGSGSGS (SEQ ID NO: 65), PSTSTST (SEQ ID NO: 66), and EIDKPSQ (SEQ ID NO: 67), and multimers thereof.

In some embodiments, the linker comprises a poly-glycine poly-serine linker. In some embodiments, the poly-glycine poly-serine linker is exclusively glycine and/or serine. In some embodiments, no more than 1, 2, 3, 4, 5, or 6 amino acids in the linker are other than glycine or serine. In some embodiments, at least 70%, 80%, 90%, or 95% of amino acids in the linker are glycine and/or serine.

### Targeting Moieties

In some embodiments, the erythroid cell comprises a targeting moiety which comprises an antibody molecule. The targeting moiety can bind a cell surface marker, e.g., a protein, present at the surface of a target cell, e.g., a cancer cell. Cell surface markers can be detected, e.g., by immunohistochemistry, immunofluorescence, FACS, or Western blot using an antibody that binds the cell surface marker.

In some embodiments, the targeting moiety comprises an antibody molecule (e.g., a scFv) fused to a transmembrane domain.

In some embodiments, the cell targeting moiety comprises an antibody molecule that binds a protein listed in Table 6. In some embodiments, the cell targeting moiety comprises an antibody molecule of Table 7.

In some embodiments, the targeting moiety (e.g., antibody molecule) comprises one or more CDRs, e.g., one or more of a heavy chain CDR1, a heavy chain CDR2, a heavy chain CDR3, a light chain CDR1, a light chain CDR2, or a light chain CDR3. In some embodiments, the targeting moiety comprises a heavy chain CDR3 (e.g., in the absence of other CDRs). In some embodiments, the antibody molecule comprises a heavy chain CDR1, a heavy chain CDR2, and a heavy chain CDR3. In some embodiments, light chain CDRs are not present. In some embodiments, the antibody molecule comprises one or more of (e.g., 2 or 3 of) a light chain CDR1, a light chain CDR2, and a light chain CDR3 (e.g., in addition to the three heavy chain CDRs).

In some embodiments, the targeting moiety (e.g., antibody molecule) comprises a heavy chain CDR1 and a light chain CDR1 of an antibody molecule of Table 7 (and optionally comprises one or more other CDRs). In some embodiments, the targeting moiety (e.g., antibody molecule) comprises a heavy chain CDR2 and a light chain CDR2 of an antibody molecule of Table 7 (and optionally comprises one or more other CDRs). In some embodiments, the targeting moiety (e.g., antibody molecule) comprises a heavy chain CDR3 and a light chain CDR3 of an antibody molecule of Table 7 (and optionally comprises one or more other CDRs).

In some embodiments, the antibody molecule comprises a heavy chain CDR1, a heavy chain CDR2, a heavy chain CDR3, a light chain CDR1, a light chain CDR2, and a light chain CDR3 of an antibody molecule of Table 7. In some embodiments, the antibody molecule comprises (a) a heavy chain CDR1, a heavy chain CDR2, a heavy chain CDR3, a light chain CDR1, a light chain CDR2, and a light chain CDR3 of an antibody molecule of Table 7, and (b) comprises a VH region having at least 70%, 75%, 80%, 85%, 90%, 95%, 96%, 97%, 98%, 99%, or 100% identity to the VH region of the antibody molecule of Table 7, and/or (c) comprises a VL region having at least 70%, 75%, 80%, 85%, 90%, 95%, 96%, 97%, 98%, 99%, or 100% identity to the VL region of the antibody molecule of Table 7. For example, the antibody molecule may have CDRs as shown in Table 7, but one or more sequence alterations in a framework region or regions.

Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. In some embodiments, the antibody molecule is or comprises an antibody fragment (e.g., antigen-binding fragment) such as an Fv fragment, a Fab fragment, a F(ab′)2 fragment, and a Fab′ fragment. Additional examples of antibody fragments include an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM). The antibody molecule may be of any class, such as IgG, IgA, or IgM (or sub-class thereof), and the antibody molecule need not be of any particular class.

In some embodiments, the antibody molecule is a multispecific antibody molecule, e.g., a bispecific antibody molecule. Examples of antibody molecules include, but are not limited to, Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, an isolated epitope binding fragment of an antibody, maxibodies, minibodies, nanobodies, intrabodies, diabodies, triabodies, tetrabodies, v-NAR and bis-scFv.

In some embodiments, the antibody molecule is a humanized antibody, a chimeric antibody, a multivalent antibody, or a fragment thereof. In some embodiments, the antibody molecule is a scFv-Fc (see, e.g., Sokolowska-Wedzina et al., Mol. Cancer Res. 15(8):1040-1050, 2017), a VHH domain (see, e.g., Li et al., Immunol. Lett. 188:89-95, 2017), a VNAR domain (see, e.g., Hasler et al., Mol. Immunol. 75:28-37, 2016), a (scFv)₂, a minibody (see, e.g., Kim et al., PLoS One 10(1):e113442, 2014), or a BiTE. In some embodiments, the antibody molecule is a DVD-Ig (see, e.g., Wu et al., Nat. Biotechnol. 25(11):1290-1297, 2007; WO 08/024188; and WO 07/024715), or a dual-affinity re-targeting antibody (DART) (Tsai et al., Mol. Ther. Oncolytics 3:15024, 2016), a triomab (see, e.g., Chelius et al., MAbs 2(3):309-319, 2010), kih IgG with a common LC (see, e.g., Kontermann et al., Drug Discovery Today 20(7):838-847, 2015), a crossmab (see, e.g., Regula et al., EMBO Mol. Med. 9(7):985, 2017), an ortho-Fab IgG, a 2-in-1-IgG, IgG-scFv (see, e.g., Cheat et al., Mol. Cancer Ther. 13(7):1803-1812, 2014), scFv2-Fc (see, e.g., Natsume et al., J. Biochem. 140(3):359-368, 2006), a bi-nanobody, tandem antibody, a DART-Fc, a scFv-HSA-scFv, a DNL-Fab3, a DAF (two-in-one or four-in-one), a DutaMab, a DT-IgG, a knobs-in-holes common LC, a knobs-in-holes assembly, a charge pair antibody, a Fab-arm exchange antibody, a SEEDbody, a Triomab, a LUZ-Y, a Fcab, a kλ-body, a orthogonal Fab, a DVD-IgG, a IgG(H)-scFv, a scFv-(H)IgG, a IgG(L)-scFv, a scFv-(L)-IgG, a IgG (L,H)-Fc, a IgG(H)-V, a V(H)—IgG, a IgG(L)-V, a V(L)-IgG, a KIH IgG-scFab, a 2scFv-IgG, a IgG-2scFv, a scFv4-Ig, a Zybody, a DVI-IgG, a nanobody (e.g., antibodies derived from Camelus bactriamus, Calelus dromaderius, or Lama paccos) (see, e.g., U.S. Pat. No. 5,759,808; and Stijlemans et al., J. Biol. Chem. 279:1256-1261, 2004; Dumoulin et al., Nature 424:783-788, 2003; and Pleschberger et al., Bioconjugate Chem. 14:440-448, 2003), a nanobody-HSA, a diabody (see, e.g., Poljak, Structure 2(12):1121-1123, 1994; and Hudson et al., J. Immunol. Methods 23(1-2):177-189, 1999), a TandAb (see, e.g., Reusch et al., mAbs 6(3):727-738, 2014), a scDiabody (see, e.g., Cuesta et al., Trends in Biotechnol. 28(7):355-362, 2010), a scDiabody-CH3 (see, e.g., Sanz et al., Trends in Immunol. 25(2):85-91, 2004), a diabody-CH3, a Triple Body, a miniantibody, a minibody, a TriBi minibody, a scFv-CH3 KIH, a Fab-scFv, a scFv-CH-CL-scFv, a F(ab′)2-scFV2, a scFv-KIH, a Fab-scFv-Fc, a tetravalent HCAb, a scDiabody-Fc, a diabody-Fc, a tandem scFv-Fc, an intrabody (see, e.g., Huston et al., Human Antibodies 10(3-4):127-142, 2001; Wheeler et al., Mol. Ther. 8(3):355-366, 2003; and Stocks, Drug Discov. Today 9(22):960-966, 2004), a dock and lock bispecific antibody, an ImmTAC, a HSAbody, a scDiabody-HSA, a tandem scFv, an IgG-IgG, a Cov-X-Body, and a scFv1-PEG-scFv2. In some embodiments, the antibody molecule can be an IgNAR, a bispecific antibody (see, e.g., Milstein and Cuello, Nature 305:537-539, 1983; Suresh et al., Methods in Enzymology 121:210, 1986; WO 96/27011; Brennan et al., Science 229:81, 1985; Shalaby et al., J. Exp. Med. 175:217-225, 1992; Kolstelny et al., J. Immunol. 148(5):1547-1553, 1992; Hollinger et al., Proc. Natl. Acad. Sci. U.S.A. 90:6444-6448, 1993; Gruber et al., J. Immunol. 152:5368, 1994; and Tuft et al., J. Immunol. 147:60, 1991), a bispecific diabody, a triabody (Schoonooghe et al., BMC Biotechnol. 9:70, 2009), a tetrabody, a scFv-Fc knobs-into-holes, a scFv-Fc-scFv, a (Fab′scFv)2, a V-IgG, a IvG-V, a dual V domain IgG, a heavy chain immunoglobulin or a camelid (Holt et al., Trends Biotechnol. 21(11):484-490, 2003), an intrabody, a heteroconjugate antibody (e.g., U.S. Pat. No. 4,676,980), a linear antibody (Zapata et al., Protein Eng. 8(10:1057-1062, 1995), a trispecific antibody (Tuft et al., J. Immunol. 147:60, 1991), a Fabs-in-Tandem immunoglobulin (WO 15/103072), or a humanized camelid antibody. In some embodiments, the antibody molecule is a synthetic antibody (also known as an antibody mimetic) (see, e.g., Yu et al. (2017) Annu. Rev. Anal. Chem. (Palo Alto Calif.) 10(1): 293-320; and Hey et al. (2005) Trends Biotechnol. 23(10): 514-22). For example, in some embodiments, the antibody molecule comprises an adnectin, an affibody, an affilin, an affimer, an affitin, an alphabody, an anticalin, an aptamer, an armadillo repeat protein-based scaffold, an atrimer, an avimer, a DARPin, a fynomer, a knottin, a Kunitz domain peptide, a monobody or a nanofitin.

### Physical Characteristics of Erythroid Cells (e.g., Enucleated Erythroid Cells)

In some embodiments, the erythroid cells described herein have one or more (e.g., 2, 3, 4, or more) physical characteristics described herein, e.g., osmotic fragility, cell size, hemoglobin concentration, or phosphatidylserine content. While not wishing to be bound by theory, in some embodiments an enucleated erythroid cell that expresses an exogenous protein has physical characteristics that resemble a wild-type, untreated erythroid cell. In contrast, a hypotonically loaded erythroid cell sometimes displays aberrant physical characteristics such as increased osmotic fragility, altered cell size, reduced hemoglobin concentration, or increased phosphatidylserine levels on the outer leaflet of the cell membrane.

In some embodiments, the enucleated erythroid cell comprises an exogenous protein that was encoded by an exogenous nucleic acid that was not retained by the cell, has not been purified, or has not existed fully outside an erythroid cell. In some embodiments, the erythroid cell is in a composition that lacks a stabilizer.

Osmotic Fragility

In some embodiments, the enucleated erythroid cell exhibits substantially the same osmotic membrane fragility as an isolated, uncultured erythroid cell that does not comprise an exogenous polypeptide. In some embodiments, the population of enucleated erythroid cells has an osmotic fragility of less than 50% cell lysis at 0.3%, 0.35%, 0.4%, 0.45%, or 0.5% NaCl. Osmotic fragility can be assayed using the method of Example 59 of WO2015/073587, which is herein incorporated by reference in its entirety.

Cell Size

In some embodiments, the enucleated erythroid cell has approximately the diameter or volume as a wild-type, untreated erythroid cell.

In some embodiments, the population of erythroid cells has an average diameter of about 4, 5, 6, 7, or 8 microns, and optionally the standard deviation of the population is less than 1, 2, or 3 microns. In some embodiments, the one or more erythroid cell has a diameter of about 4-8, 5-7, or about 6 microns. In some embodiments, the diameter of the erythroid cell is less than about 1 micron, larger than about 20 microns, between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns or between about 10 microns and about 30 microns. Cell diameter is measured, in some embodiments, using an Advia 120 hematology system.

In some embodiment the volume of the mean corpuscular volume of the erythroid cells is greater than 10 fL, 20 fL, 30 fL, 40 fL, 50 fL, 60 fL, 70 fL, 80 fL, 90 fL, 100 fL, 110 fL, 120 fL, 130 fL, 140 fL, 150 fL, or greater than 150 fL. In one embodiment the mean corpuscular volume of the erythroid cells is less than 30 fL, 40 fL, 50 fL, 60 fL, 70 fL, 80 fL, 90 fL, 100 fL, 110 fL, 120 fL, 130 fL, 140 fL, 150 fL, 160 fL, 170 fL, 180 fL, 190 fL, 200 fL, or less than 200 fL. In one embodiment the mean corpuscular volume of the erythroid cells is between 80-100, 100-200, 200-300, 300-400, or 400-500 femtoliters (fL). In some embodiments, a population of erythroid cells has a mean corpuscular volume set out in this paragraph and the standard deviation of the population is less than 50, 40, 30, 20, 10, 5, or 2 fL. The mean corpuscular volume is measured, in some embodiments, using a hematological analysis instrument, e.g., a Coulter counter.

Hemoglobin Concentration

In some embodiments, the enucleated erythroid cell has a hemoglobin content similar to a wild-type, untreated erythroid cell. In some embodiments, the erythroid cells comprise greater than 1%, 2%, 3%, 4%, 5%, 6%, 7%, 8%, 9% or greater than 10% fetal hemoglobin. In some embodiments, the erythroid cells comprise at least about 20, 22, 24, 26, 28, or 30 pg, and optionally up to about 30 pg, of total hemoglobin. Hemoglobin levels are determined, in some embodiments, using the Drabkin's reagent method of Example 33 of WO2015/073587, which is herein incorporated by reference in its entirety.

Phosphatidylserine Content

In some embodiments, the enucleated erythroid cell has approximately the same phosphatidylserine content on the outer leaflet of its cell membrane as a wild-type, untreated erythroid cell. Phosphatidylserine is predominantly on the inner leaflet of the cell membrane of wild-type, untreated erythroid cells, and hypotonic loading can cause the phosphatidylserine to distribute to the outer leaflet where it can trigger an immune response. In some embodiments, the population of erythroid cells comprises less than about 30, 25, 20, 15, 10, 9, 8, 6, 5, 4, 3, 2, or 1% of cells that are positive for Annexin V staining. Phosphatidylserine exposure is assessed, in some embodiments, by staining for Annexin-V-FITC, which binds preferentially to PS, and measuring FITC fluorescence by flow cytometry, e.g., using the method of Example 54 of WO2015/073587, which is herein incorporated by reference in its entirety.

Other Characteristics

In some embodiments, the population of erythroid cells comprises at least about 50%, 60%, 70%, 80%, 90%, or 95% (and optionally up to 90 or 100%) of cells that are positive for GPA. The presence of GPA is detected, in some embodiments, using FACS.

In some embodiments, the erythroid cells have a half-life of at least 0.5, 1, 2, 7, 14, 30, 45, or 90 days in a subject.

In some embodiments, a population of cells comprising erythroid cells comprises less than about 10, 5, 4, 3, 2, or 1% echinocytes.

In some embodiments, an erythroid cell is enucleated, e.g., a population of cells comprising erythroid cells used as a therapeutic preparation described herein is greater than 50%, 60%, 70%, 80%, 90% enucleated. In some embodiments, a cell, e.g., an erythroid cell, contains a nucleus that is non-functional, e.g., has been inactivated.

In some embodiments, an erythroid cell or population of cells comprises one or more of (e.g., all of) endogenous GPA, band3, or alpha4 integrin. These proteins can be measured, e.g., as described in Example 10 of International Application Publication No. WO2018/009838, which is herein incorporated by reference in its entirety. The percentage of GPA-positive cells and Band3-positive cells typically rises during maturation of an erythroid cell, and the percentage of Alpha4 integrin-positive typically remains high throughout maturation.

Universal Donor Erythroid Cells

In some embodiments, erythroid cells described herein are autologous and/or allogeneic to the subject to which the cells will be administered. For example, erythroid cells allogeneic to the subject include one or more of blood type specific erythroid cells (e.g., the cells can be of the same blood type as the subject) or one or more universal donor erythroid cells. In some embodiments, the enucleated erythroid cells described herein have reduced immunogenicity compared to a reference cell, e.g., have lowered levels of one or more blood group antigens.

Where allogeneic cells are used for transfusion, a compatible ABO blood group can be chosen to prevent an acute intravascular hemolytic transfusion reaction. The ABO blood types are defined based on the presence or absence of the blood type antigens A and B, monosaccharide carbohydrate structures that are found at the termini of oligosaccharide chains associated with glycoproteins and glycolipids on the surface of the erythrocytes (reviewed in Liu et al., Nat. Biotech. 25:454-464 (2007)). Because group O erythrocytes contain neither A nor B antigens, they can be safely transfused into recipients of any ABO blood group, e.g., group A, B, AB, or O recipients. Group O erythrocytes are considered universal and may be used in all blood transfusions. Thus, in some embodiments, an erythroid cell described herein is type O. In contrast, group A erythroid cells may be given to group A and AB recipients, group B erythroid cells may be given to group B and AB recipients, and group AB erythroid cells may be given to AB recipients.

In some instances, it may be beneficial to convert a non-group O erythroid cell to a universal blood type. Enzymatic removal of the immunodominant monosaccharides on the surface of group A and group B erythrocytes may be used to generate a population of group O-like erythroid cells (See, e.g., Liu et al., Nat. Biotech. 25:454-464 (2007)). Group B erythroid cells may be converted using an α-galactosidase from green coffee beans. Alternatively or in addition, α-N-acetylgalactosaminidase and α-galactosidase enzymatic activities from E. meningosepticum bacteria may be used to respectively remove the immunodominant A and B antigens (Liu et al., Nat. Biotech. 25:454-464 (2007)), if present on the erythroid cells. In one example, packed erythroid cells isolated as described herein, are incubated in 200 mM glycine (pH 6.8) and 3 mM NaCl in the presence of either α-N-acetylgalactosaminidase and α-galactosidase (about 300 μg/ml packed erythroid cells) for 60 min at 26° C. After treatment, the erythroid cells are washed by 3-4 rinses in saline with centrifugation and ABO-typed according to standard blood banking techniques.

While the ABO blood group system is the most important in transfusion and transplantation, in some embodiments it can be useful to match other blood groups between the erythroid cells to be administered and the recipient, or to select or make erythroid cells that are universal for one or more other (e.g., minor) blood groups. A second blood group is the Rh system, wherein an individual can be Rh+ or Rh−. Thus, in some embodiments, an erythroid cell described herein is Rh−. In some embodiments, the erythroid cell is Type O and Rh−.

In some embodiments, an erythroid cell described herein is negative for one or more minor blood group antigens, e.g., Le(a-b-) (for Lewis antigen system), Fy(a-b-) (for Duffy system), Jk(a-b-) (for Kidd system), M-N- (for MNS system), K-k- (for Kell system), Lu(a-b-) (for Lutheran system), and H-antigen negative (Bombay phenotype), or any combination thereof. In some embodiments, the erythroid cell is also Type 0 and/or Rh−. Minor blood groups are described, e.g., in Agarwal et al “Blood group phenotype frequencies in blood donors from a tertiary care hospital in north India” Blood Res. 2013 March; 48(1): 51-54 and Mitra et al “Blood groups systems” Indian J Anaesth. 2014 September-October; 58(5): 524-528, each of which is incorporated herein by reference in its entirety.

### Methods of Manufacturing Enucleated Erythroid Cells

Methods of manufacturing enucleated erythroid cells comprising (e.g., expressing) an exogenous agent (e.g., polypeptides) are described, e.g., in WO2015/073587 and WO2015/153102, each of which is incorporated by reference in its entirety.

In some embodiments, hematopoietic progenitor cells, e.g., CD34+ hematopoietic progenitor cells (e.g., human or mouse cells), are contacted with a nucleic acid or nucleic acids encoding one or more exogenous polypeptides, and the cells are allowed to expand and differentiate in culture. In some embodiments, the CD34+ cells are immortalized, e.g., comprise a human papilloma virus (HPV; e.g., HPV type 16) E6 and/or E7 genes. In some embodiments, the immortalized CD34+ hematopoietic progenitor cell is a BEL-A cell line cell (see Trakarnasanga et al. (2017) Nat Commun. 8: 14750). Additional immortalized CD34+ hematopoietic progenitor cells are described in U.S. Pat. Nos. 9,951,350, and 8,975,072. In some embodiments, an immortalized CD34+ hematopoietic progenitor cell is contacted with a nucleic acid or nucleic acids encoding one or more exogenous polypeptides, and the cells are allowed to expand and differentiate in culture.

In some embodiments, the erythroid cells described herein are made by a method comprising contacting a nucleated erythroid cell, or precursor thereof, with an exogenous nucleic acid. The exogenous nucleic acid may be a nucleic acid that is not produced by a wild-type cell of that type or is present at a lower level in a wild-type cell than in a cell containing the exogenous nucleic acid. In some embodiments, the exogenous nucleic acid is codon-optimized. For instance, the exogenous nucleic acid may comprise one or more codons that differ from the wild-type codons in a way that does not change the amino acid encoded by that codon, but that increases translation of the nucleic acid, e.g., by using a codon preferred by the host cell, e.g., a mammalian cell, e.g., an erythroid cell.

The method may further comprise culturing the nucleated erythroid cell, or precursor thereof, under conditions suitable for expression of the exogenous protein and/or for enucleation.

In some embodiments, the two or more polypeptides (e.g., a first exogenous polypeptide comprising an amino acid degradative enzyme and a second exogenous polypeptide comprising a cell targeting moiety) are encoded in a single nucleic acid, e.g. a single vector. In some embodiments, the single vector has a separate promoter for each gene. In some embodiments, the single vector includes a single nucleic acid (e.g., a single open reading frame) encoding a fusion protein including at least two polypeptides and a protease cleavage site disposed between the first polypeptide and the second polypeptide. This fusion protein may be initially expressed as a single polypeptide that subsequently may be proteolytically processed by a protease capable of recognizing and cleaving at the protease cleavage site to thereby yield the two polypeptides. The single vector may also encode the two or more polypeptides in any other suitable configuration. In some embodiments, the two or more polypeptides are encoded by two or more nucleic acids, e.g., each vector encodes one of the polypeptides.

The nucleic acid may be, e.g., DNA or RNA (e.g., mRNA). A number of viruses may be used as gene transfer vehicles including retroviruses, Moloney murine leukemia virus (MMLV), adenovirus, adeno-associated virus (AAV), herpes simplex virus (HSV), lentiviruses such as human immunodeficiency virus 1 (HIV 1), and spumaviruses such as foamy viruses, for example.

In some embodiments, the exogenous nucleic acid is operatively linked to a constitutive promoter. In some embodiments, a constitutive promoter is used to drive expression of the targeting moiety.

In some embodiments, the exogenous nucleic acid is operatively linked to an inducible or repressible promoter, e.g., to drive expression of the amino acid degradative enzyme. For instance, the promoter may be doxycycline-inducible, e.g., a P-TRE3GS promoter or active fragment or variant thereof. Examples of inducible promoters include, but are not limited to a metallothionine-inducible promoter, a glucocorticoid-inducible promoter, a progesterone-inducible promoter, and a tetracycline-inducible promoter (which may also be doxycycline-inducible). In some embodiments, the inducer is added to culture media comprising cells that comprise the inducible promoter, e.g., at a specific stage of cell differentiation. In some embodiments, the inducer (e.g., doxycycline) is added at an amount of about 1-5, 2-4, or 3 μg/mL. In some embodiments, a repressor is withdrawn from to culture media comprising cells that comprise the repressible promoter, e.g., at a specific stage of cell differentiation. In some embodiments, the inducer is added, or the repressor is withdrawn, during maturation phase, e.g., between days 1-10, 2-9, 3-8, 4-6, or about day 5 of maturation phase. In some embodiments, the inducer is present, or the repressor is absent, between day 1, 2, 3, 4, 5, 6, 7, 8, 9 or 10 of maturation and enucleation. In some embodiments, the inducer is present, or the repressor is absent, for at least 1, 2, 3, 4, 5, 6, 7, 8, 9, or 10 days. In some embodiments, the inducer is present, or the repressor is absent, from maturation day 5 to the end of differentiation. In embodiments, the inducer is present, or the repressor is absent at maturation day 9. In some embodiments, the inducer is added, or the repressor is withdrawn, when the population of erythroid cells comprises a plurality of normoblasts (e.g., basophilic, polychromatic, or orthochromatic normoblasts or a combination thereof), e.g., when 10-20%, 20-30%, 30-40%, 40-50%, 50-60%, 60-70%, or 70-80% of the cells in the population are normoblasts. In some embodiments, the inducer is added, or the repressor is withdrawn, when the population of erythroid cells comprises a plurality of pro-erythroblasts, e.g., when 10-20%, 20-30%, 30-40%, 40-50%, 50-60%, 60-70%, or 70-80% of the cells in the population are pro-erythroblasts. In some embodiments, the inducer is added, or the repressor is withdrawn, when the population of erythroid cells comprises a plurality of erythroblasts at terminal differentiation e.g., when 10-20%, 20-30%, 30-40%, 40-50%, 50-60%, 60-70%, or 70-80% of the cells in the population are erythroblasts at terminal differentiation. In some embodiments, the erythroid cell or population of erythroid cells comprises an additional exogenous protein, e.g., a transactivator, e.g., a Tet-inducible transactivator (e.g., a Tet-on-3G transactivator).

In some embodiments, the inducer is added, or the repressor is withdrawn, when the population of erythroid cells comprises one or more of (e.g., all of) endogenous GPA, band3, or alpha4 integrin. In some embodiments, the inducer is added, or the repressor is withdrawn, during a time when about 84-100%, 85-100%, 90-100%, or 95-100% of the cells in the population are GPA-positive (e.g., when the population first reaches that level); during a time when 50-100%, 60-100%, 70-100%, 80-100%, 90-100%, 95-100%, or 98-100% of the cells in the population are band3-positive (e.g., when the population first reaches that level); and/or during a time when about 70-100%, 80-90%, or about 85% of the cells in the population are alpha4 integrin-positive (e.g., when the population first reaches that level).

GPA, band3, and alpha4 integrin can be detected, e.g., by a flow cytometry assay, e.g., a flow cytometry assay of Example 10 of International Application Publication No. WO2018/009838.

In some embodiments, the cells are produced using conjugation, e.g., sortagging or sortase-mediated conjugation, e.g., as described in International Application Publication Nos. WO2014/183071 or WO2014/183066, each of which is incorporated by reference in its entirety. In some embodiments, the cells are made by a method that does not comprise sortase-mediated conjunction.

In some embodiments, the cells are made by a method that does not comprise hypotonic loading. In some embodiments, the cells are made by a method that does not comprise a hypotonic dialysis step.

In some embodiments, the erythroid cells are expanded at least 1000, 2000, 5000, 10,000, 20,000, 50,000, or 100,000 fold (and optionally up to 100,000, 200,000, or 500,000 fold). The number of cells is measured, in some embodiments, using an automated cell counter.

In some embodiments, the population of erythroid cells comprises at least about 30%, 35%, 40%, 45%, 50%, 55%, 60%, 65%, 70%, 75%, 80%, 85%, 90%, 95%, 96% or 98% (and optionally up to about 80, 90, or 100%) enucleated erythroid cells. In some embodiments, the population of erythroid cells comprises 70%-100%, 75%-100%, 80%-100%, 85%-100%, or 90%-100% enucleated cells. In some embodiments, the population of erythroid cells contains less than 1% live nucleated cells, e.g., contains no detectable live nucleated cells. Enucleation is measured, in some embodiments, by FACS using a nuclear stain. In some embodiments, at least about 30%, 35%, 40%, 45%, 50%, 55%, 60%, 65%, 70%, 75%, 80%, 85%, 90%, 95%, 96% or 98% (and optionally up to about 70, 80, 90, or 100%) of erythroid cells in the population comprise one or more (e.g., 2, 3, 4 or more) of the exogenous polypeptides. Expression of the polypeptides is measured, in some embodiments, by erythroid cells using labeled antibodies against the polypeptides. In some embodiments, at least about 30%, 35%, 40%, 45%, 50%, 55%, 60%, 65%, 70%, 75%, 80%, 85%, 90%, 95%, 96% or 98% (and optionally up to about 70, 80, 90, or 100%) of erythroid cells in the population are enucleated and comprise one or more (e.g., 2, 3, 4, or more) of the exogenous polypeptides. In some embodiments, the population of erythroid cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

### Vehicles for Polypeptides Described Herein

While in many embodiments herein, the one or more (e.g., two or more) exogenous polypeptides are situated on or in an enucleated erythroid cell, it is understood that any polypeptide or combination of exogenous polypeptides described herein can also be situated on or in another vehicle. The vehicle can comprise, e.g., a cell, an erythroid cell, a corpuscle, a nanoparticle, a micelle, a liposome, or an exosome. For instance, in some aspects, the present disclosure provides a vehicle (e.g., a cell, an erythroid cell, a corpuscle, a nanoparticle, a micelle, a liposome, or an exosome) comprising, e.g., on its surface, one or more agents described herein. In some embodiments, the one or more agents comprise an agent selected from a polypeptide of any of Tables 1-7 or a fragment or variant thereof, or an antibody molecule thereto. In some embodiments, the vehicle comprises two or more agents described herein, e.g., any pair of agents described herein.

In some embodiments, the vehicle comprises an erythroid cell. In some embodiments, the erythroid cell is a nucleated red blood cell, red blood cell precursor, or enucleated red blood cell. In some embodiments, the erythroid cell is a cord blood stem cell, a CD34+ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), a reticulocyte, an erythrocyte, an induced pluripotent stem cell (iPSC), a mesenchymal stem cell (MSC), a polychromatic normoblast, an orthochromatic normoblast, or a combination thereof. In some embodiments, the erythroid cells are immortal or immortalized cells.

### Heterogeneous Populations of Cells

While in many embodiments herein, the one or more (e.g., two or more) exogenous polypeptides are situated on or in a single cell, it is understood that any polypeptide or combination of polypeptides described herein can also be situated on a plurality of cells. For instance, in some aspects, the disclosure provides a plurality of erythroid cells, wherein a first cell of the plurality comprises a first exogenous polypeptide (e.g., comprising an amino acid degradative enzyme described herein) and a second cell of the plurality comprises a second exogenous polypeptide (e.g., comprising a cell targeting moiety described herein). In some embodiments, the plurality of cells comprises two or more polypeptides described herein, e.g., any pair of polypeptides described herein. In some embodiments, less than 90%, 80%, 70%, 60%, 50%, 40%, 30%, 20%, 10%, 5%, 2%, or 1% of the cells in the population comprise both the first exogenous polypeptide and the second exogenous polypeptide.

### Cells Encapsulated in a Membrane

In some embodiments, enucleated erythroid cells or other vehicles described herein are encapsulated in a membrane, e.g., semi-permeable membrane. In some embodiments, the membrane comprises a polysaccharide, e.g., an anionic polysaccharide alginate. In some embodiments, the semipermeable membrane does not allow cells to pass through, but allows passage of small molecules or macromolecules, e.g., metabolites, proteins, or DNA. In some embodiments, the membrane is one described in Lienert et al., “Synthetic biology in mammalian cells: next generation research tools and therapeutics” Nature Reviews Molecular Cell Biology 15, 95-107 (2014), incorporated herein by reference in its entirety. While not wishing to be bound by theory, in some embodiments, the membrane shields the cells from the immune system and/or keeps a plurality of cells in proximity, facilitating interaction with each other or each other's products.

### Articles of Manufacture

In some embodiments, a plurality of erythroid cells described herein is provided in an article of manufacture (e.g., a container or medical device). In some embodiments, the article of manufacture comprises a container (e.g., a vial, e.g., comprising glass or plastic). In some embodiments, the article of manufacture is a medical device (e.g., a catheter or a syringe). In some embodiments, the container comprises a single dose of the erythroid cells, e.g., about 5×10⁹-1×10¹⁰, 1-2×10¹⁰, 2-5×10¹⁰, or 5×10¹⁰-1×10¹¹ cells. In other embodiments, the container may comprise a plurality of doses.

Methods of Treatment with Compositions Herein, e.g., Enucleated Erythroid Cells

Methods of administering enucleated erythroid cells (e.g., reticulocytes) comprising (e.g., expressing) exogenous agent (e.g., polypeptides) are described, e.g., in WO2015/073587 and WO2015/153102, each of which is incorporated by reference in its entirety.

In some embodiments, the enucleated erythroid cells described herein are administered to a subject, e.g., a mammal, e.g., a human. Exemplary mammals that can be treated include without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like). The methods described herein are applicable to both human therapy and veterinary applications. The subject may be, for example, an adult or a child. In some embodiments, the subject is a human subject between the ages of 0-18 years, 18-65 years, or over 65 years old.

In some embodiments, the erythroid cells are administered to a patient every 1, 2, 3, 4, 5, or 6 months.

In some embodiments, a dose of erythroid cells comprises about 1×10⁹-2×10⁹, 2×10⁹, 5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹, 5×10¹¹, 5×10¹¹-1×10¹², 1×10¹²-2×10¹², 2×10¹²-5×10¹², or 5×10¹²-1×10¹³ cells.

In some embodiments, the erythroid cells are administered to a patient in a dosing regimen (dose and periodicity of administration) sufficient to maintain function of the administered erythroid cells in the bloodstream of the patient over a period of 2 weeks to a year, e.g., one month to one year or longer, e.g., at least 2 weeks, 4 weeks, 6 weeks, 8 weeks, 3 months, 6 months, a year, 2 years.

In some aspects, the present disclosure provides a method of treating a disease or condition described herein, comprising administering to a subject in need thereof a composition described herein, e.g., an enucleated erythroid cell described herein. In some embodiments, the disease or condition is a cancer, e.g., leukemia. In some embodiments, the cancer is chosen from acute lymphoblastic leukaemia (ALL), an acute myeloid leukaemia (AML), an anal cancer, a bile duct cancer, a bladder cancer, a bone cancer, a bowel cancer, a brain tumor, a breast cancer, a carcinoid, a cervical cancer, a choriocarcinoma, a chronic lymphocytic leukaemia (CLL), a chronic myeloid leukaemia (CML), a colon cancer, a colorectal cancer, an endometrial cancer, an eye cancer, a gallbladder cancer, a gastric cancer, a gestational trophoblastic tumor (GTT), a hairy cell leukaemia, a head and neck cancer, a Hodgkin lymphoma, a kidney cancer, a laryngeal cancer, a liver cancer, a lung cancer, a lymphoma, a melanoma, a skin cancer, a mesothelioma, a mouth or oropharyngeal cancer, a myeloma, a nasal or sinus cancer, a nasopharyngeal cancer, a non-Hodgkin lymphoma (NHL), an oesophageal cancer, an ovarian cancer, a pancreatic cancer, a penile cancer, a prostate cancer, a rectal cancer, a salivary gland cancer, a non-melanoma skin cancer, a soft tissue sarcoma, a stomach cancer, a testicular cancer, a thyroid cancer, a uterine cancer, a vaginal cancer, and a vulval cancer.

In some embodiments, cancer cells of the subject are auxotrophic, e.g., at least a sub-population of cancer cells in the subject are auxotrophic. In some embodiments, one or more cancer cells in the subject have impaired synthesis of an amino acid, e.g, asparagine and/or glutamine. In some embodiments, the cancer has a mutation in an amino acid synthesis gene, e.g., wherein the mutation reduces or eliminates activity of the gene product. In some embodiments, the amino acid synthesis gene encodes a protein that contributes to biosynthesis of the amino acid, e.g., catalyzes formation of the amino acid from a precursor molecule.

In some embodiments, the enucleated erythroid cell described herein comprises an asparaginase molecule and an anti-CD33 targeting moiety, and is used for the treatment of cancer (e.g., leukemia, e.g., ALL or CLL).

In some embodiments, the enucleated erythroid cell described herein is administered as together with a second therapy. The second therapy may comprise, e.g., chemotherapy, radiation therapy, surgery, or an antibody therapy.

The erythroid cells described herein may be administered through any suitable route of administration. In some embodiments, intravenous administration is used. In some embodiments, the erythroid cells (e.g., nucleated or enucleated erythroid cells) are administered such that at least a subset of the erythroid cells reaches the bone marrow of the subject. The cells may be administered directly to the bone marrow.

In some embodiments, an erythroid cell described herein has anti-cancer activity, e.g., as measured by a method described herein.

Efficacy can be assayed, for example, by contacting an erythroid cell described herein with a cancer cell in vitro, and assaying one or more of the following: number of cancer cells, division rate of cancer cells, and replication of cancer cell DNA. See, e.g., Example 9 herein for suitable reaction conditions. In some embodiments, the method comprises contacting the erythroid cell (e.g., comprising about 0.1 μg, 0.5 μg, or 1 μg of the amino acid degradative enzyme) with cancer cells (e.g., one or more of MV4-11, MOLM-13, THP1, HL60, B16-F10, RPMI 8226), and optionally with CD34+ hematopoietic stem cells as a control. The number of live cells can be determined after incubation, e.g., for 68 or 87 hours. In some embodiments, the percentage of live cancer cells remaining after the incubation period is less than 50%, 40%, 30%, 20%, or 10%, e.g., between 10%-50%, 10%-40%, 10%, 30%, or 10%-20%.

Anti-cancer efficacy can also be assayed in an animal model, e.g., as described in Example 8. For instance, erythroid cells as described herein can be administered to a mouse cancer model, e.g., a disseminated MV4-11 AML mouse model, e.g., an NSG mouse injected with human AML MV4-11 cells, e.g., at a dose of 2×10⁶ cells, and allowed to grow or engraft, e.g., for 21 or 24 days and/or until tumor load in peripheral blood is about 0.5%-2%. The number of cancer cells in the blood (e.g., a 35 ul sample) may be measured, e.g., 4 days after dosing. In some embodiments, the number of cancer cells is less than 600, 500, 400, 300, 200, or 100 per sample. In some embodiments, the number of cancer cells is less than 80%, 70%, 60%, 50%, 40%, 30%, 20%, or 10% of the level of cancer cells in a control mouse, e.g., a mouse treated with control cells that lack the exogenous amino acid degradative enzyme.

In addition, amino acid degradative activity can be assayed in an animal model, e.g., as described in Example 7. For example, erythroid cells as described herein can be administered to a mouse, e.g., a NOD SCID mouse; a sample (e.g., a blood sample or plasma sample) can be taken from the mouse, and amino acid levels can be measured in the sample. In some embodiments, the serum level of the amino acid (e.g., asparagine or glutamine) is below 60, 50, 40, 30, 20, or 10 μM, e.g., about 2, 4, 6, or 8 days after dosing. In some embodiments, the serum level of the amino acid (e.g., asparagine or glutamine) is less than 80%, 70%, 60%, 50%, 40%, 30%, 20%, or 10% of the level of the amino acid in a control mouse, e.g., a mouse treated with control cells that lack the exogenous amino acid degradative enzyme.

### Tables

TABLE 1
Exemplary amino acid sequences of polypeptides comprising
an amino acid degradative enzyme or a cell targeting moiety
(e.g., Erwinia chrysanthemi asparaginase molecules fused to
Kell and anti-CD33 scFv fused to Glycophorin A).

| SEQ ID | | |
|---|---|---|
| NO: | Sequence Name | Amino acid sequence |
| 1 | Erwinia | MEGGDQSEEEPRERSQAGGMGTLWSQESTPEERL |
| | chrysanthemi | PVEGSRPWAVARRVLTAILILGLLLCFSVLLFYNFQ |
| | asparaginase | NCGPRPCET [symbol] [symbol] [symbol] [symbol] |
| | fused to the C- | [symbol] ADKLPNIVI LATGGTIAGS AATGTQTTGY |
| | terminus of amino | KAGALGVDTL INAVPEVKKL ANVKGEQFSN |
| | acid residues 1-79 | MASENMTGDV VLKLSQRVNELLARDDVDGV |
| | of Kell | VITHGTDTVE ESAYFLHLTV KSDKPVVFVA |
| | (Kell-HA- | AMRPATAISADGPMNLLEAV RVAGDKQSRG |
| | ErwASNase; | RGVMVVLNDR IGSARYITKT |
| | linker shown in | NASTLDTFKANEEGYLGVII GNRIYYQNRI |
| | italics and | DKLHTTRSVF DVRGLTSLPK |
| | underlined) | VDILYGYQDDPEYLYDAAIQ HGVKGIVYAG |
| | | MGAGSVSVRG IAGMRKAMEK |
| | | GVVVIRSTRTGNGIVPPDEE LPGLVSDSLN |
| | | PAHARILLML ALTRTSDPKV IQEYFHTY |
| 2 | anti-CD33 scFv | MYGKIIFVLLLSEIVSISALLSEIVSISAQVQLVQSGA |
| | fused to the N- | EVKKPGASVKVSCKASGYTFTNYDINWVRQAPGQ |
| | terminus of GPA | GLEWIGWIYPGDGSTKYNEKFKAKATLTADTSTST |
| | (αCD33scFv- | AYMELRSLRSDDTAVYYCASGYEDAMDYWGQGT |
| | GPA; linker | TVTVSS [symbol] [symbol] DIQMTQSPSSLSAS |
| | regions shown in | VGDRVTINCKASQDINSYLSWFQQKPGKAPKTLIY |
| | italics and | RANRLVDGVPSRFSGSGSGQDYTLTISSLQPEDFAT |
| | underlined) | YYCLQYDEFPLTFGGGTKVEIK [symbol] YPYDV |
| | | PDYA [symbol] MYGKIIFVLLLSEIVSISALSTTEV |
| | | AMHTSTSSSVTKSYISSQTNDTHKRDTYAATPRAH |
| | | EVSEISVRTVYPPEEETGERVQLAHHFSEPEITLIIFG |
| | | VMAGVIGTILLISYGIRRLIKKSPSDVKPLPSPDTDV |
| | | PLSSVEIENPETSDQ |
| 54 | Erwinia | MQPQESHVHYSRWEDGSRDGVSLGAVSSTEEASRCRRISQ |
| | chrysanthemi | RLCTGKLGIAMKVLGGVALFWIIFILGYLTGYYVHKCK |
| | asparaginase | GGGGSGGGGSGGGGSGGGGSGGGGSGGGGS |
| | fused to the C- | ADKLPNIVI LATGGTIAGS AATGTQTTGY |
| | terminus of | KAGALGVDTL INAVPEVKKL ANVKGEQFSN |
| | SMIM1 | MASENMTGDV VLKLSQRVNE LLARDDVDGV |
| | | VITHGTDTVE ESAYFLHLTV KSDKPVVFVA |
| | | AMRPATAISA DGPMNLLEAV RVAGDKQSRG |
| | | RGVMVVLNDR IGSARYITKT NASTLDTFKA |
| | | NEEGYLGVII GNRIYYQNRI DKLHTTRSVF |
| | | DVRGLTSLPK VDILYGYQDD PEYLYDAAIQ |
| | | HGVKGIVYAG MGAGSVSVRG IAGMRKAMEK |
| | | GVVVIRSTRT |
| | | GNGIVPPDEE LPGLVSDSLN PAHARILLML |
| | | ALTRTSDPKV IQEYFHTY |
| 55 | Anti-CD33 scFv | MYGKIIFVLLLSEIVSISA |
| | fused to the N- | EVQLVQSGAEVKKPGSSVKVSCKASGYTITDSNIHWVR |
| | terminus of GPA | QAPGQSLEWIGYIYPYNGGTDYNQKFKNRATLTVDNPT |
| | | NTAYMELSSLRSEDTAFYYCVNGNPWLAYWGQGTLVTV |
| | | SS |
| | | GGGGSGGGGSGGGGS |
| | | DIQLTQSPSTLSASVGDRVTITCRASESLDNYGIRFLT |
| | | WFQQKPGKAPKLLMYAASNQGSGVPSRFSGSGSGTEFT |
| | | LTISSLQPDDFATYYCQQTKEVPWSFGQGTKVEVK |
| | | GGGGSGGGGSGGGGSGGGGSGGGGSGGGG |
| | | LSTTEVAMHTSTSSSVTKSYISSQTNDTHKRDTYAATPRAH |
| | | EVSEISVRTVYPPEEETGERVQLAHHFSEPEITLIIFGVMA |
| | | GVIGTILLISYGIRRLIKKSPSDVKPLPSPDTDVPLSSVEI |
| | | ENPETSDQ |
| 114 | Pseudomonas 7A | MQPQESHVHYSRWEDGSRDGVSLGAVSSTEEASRCRRISQR |
| | glutaminase | LCTGKLGIAMKVLGGVALFWIIFILGYLTGYYVHKCK |
| | asparaginase | GGGGSGGGGSGGGGSGGGGSGGGGSGGGGS |
| | fused to the C- | KEVENQQKLANVVILATGGTIAGAGASAANSATYQAAKVGV |
| | terminus of | DKLIAGVPELADLANVRGEQVMQIASESITNDDLLKLGKRV |
| | SMIM1 via a | AELADSNDVDGIVITHGTDTLEETAYFLNLVEKTDKPIVVV |
| | (Gly₄Ser)₆ linker | GSMRPGTAMSADGMLNLYNAVAVASNKDSRGKGVLVTMNDE |
| | (underlined) | IQSGRDVSKSINIKTEAFKSAWGPLGMVVEGKSYWFRLPAK |
| | | RHTVNSEFDIKQISSLPQVDIAYSYGNVTDTAYKALAQNGA |
| | | KALIHAGTGNGSVSSRVVPALQELRKNGVQIIRSSHVNQGG |
| | | FVLRNAEQPDDKNDWVVAHDLNPQKARILAMVAMTKTQDSK |
| | | ELQRIFWEY |
| 115 | Pseudomonas 7A | MQPQESHVHYSRWEDGSRDGVSLGAVSSTEEASRCRRISQR |
| | glutaminase | LCTGKLGIAMKVLGGVALFWIIFILGYLTGYYVHKCK |
| | asparaginase | GGGGSGGGGSYPYDVPDYAGGGGSGGGGS |
| | fused to the C- | KEVENQQKLANVVILATGGTIAGAGASAANSATYQAAKVGV |
| | terminus of | DKLIAGVPELADLANVRGEQVMQIASESITNDDLLKLGKRV |
| | SMIM1 via a | AELADSNDVDGIVITHGTDTLEETAYFLNLVEKTDKPIVVV |
| | linker | GSMRPGTAMSADGMLNLYNAVAVASNKDSRGKGVLVTMNDE |
| | (underlined) | IQSGRDVSKSINIKTEAFKSAWGPLGMVVEGKSYWFRLPAK |
| | containing the | RHTVNSEFDIKQISSLPQVDIAYSYGNVTDTAYKALAQNGA |
| | HA epitope tag | KALIHAGTGNGSVSSRVVPALQELRKNGVQIIRSSHVNQGG |
| | (SMIM1-HA- | FVLRNAEQPDDKNDWVVAHDLNPQKARILAMVAMTKTQDSK |
| | PseuGLNase) | ELQRIFWEY |

TABLE 2
Exemplary amino acid sequences and activities of asparaginase molecules
from various species.

| SEQ | Sequence | | | |
|---|---|---|---|---|
| ID | name and | | Asparaginase | Glutaminase |
| NO: | source | Amino acid sequence | activity | activity |
| 3 | Erwinia | MERWFKSLFV LVLFFVFTAS | Kₘ = 0.058-0.080 mM | Kₘ = 1.7-6.7 mM |
| | chrysanthemi | AADKLPNIVI LATGGTIAGS | k_{cat} = 397-440 s⁻¹ | k_{cat} = 65-72 s⁻¹ |
| | L- | AATGTQTTGY KAGALGVDTL | | |
| | asparaginase | INAVPEVKKL ANVKGEQFSN | | |
| | UniProt | MASENMTGDV | | |
| | P06608 | VLKLSQRVNE LLARDDVDGV | | |
| | | VITHGTDTVE ESAYFLHLTV | | |
| | | KSDKPVVFVA AMRPATAISA | | |
| | | DGPMNLLEAV | | |
| | | RVAGDKQSRG | | |
| | | RGVMVVLNDR IGSARYITKT | | |
| | | NASTLDTFKA NEEGYLGVII | | |
| | | GNRIYYQNRI DKLHTTRSVF | | |
| | | DVRGLTSLPK VDILYGYQDD | | |
| | | PEYLYDAAIQ HGVKGIVYAG | | |
| | | MGAGSVSVRG | | |
| | | IAGMRKAMEK GVVVIRSTRT | | |
| | | GNGIVPPDEE LPGLVSDSLN | | |
| | | PAHARILLML ALTRTSDPKV | | |
| | | IQEYFHTY | | |
| 4 | Escherichia | MEFFKKTALA ALVMGFSGAA | Kₘ = 0.015 mM | Kₘ = 3.5 mM |
| | coli L- | LALPNITILA TGGTIAGGGD | k_{cat} = 24 s⁻¹ | k_{cat} = 0.33 s⁻¹ |
| | asparaginase 2 | SATKSNYTVG KVGVENLVNA | | |
| | UniProt | VPQLKDIANV KGEQVVNIGS | | |
| | P00805 | QDMNDNVWLT | | |
| | | LAKKINTDCD KTDGFVITHG | | |
| | | TDTMEETAYF LDLTVKCDKP | | |
| | | VVMVGAMRPS TSMSADGPFN | | |
| | | LYNAVVTAAD | | |
| | | KASANRGVLV | | |
| | | VMNDTVLDGR | | |
| | | DVTKTNTTDV ATFKSVNYGP | | |
| | | LGYIHNGKID YQRTPARKHT | | |
| | | SDTPFDVSKL NELPKVGIVY | | |
| | | NYANASDLPA | | |
| | | KALVDAGYDG IVSAGVGNGN | | |
| | | LYKSVFDTLA TAAKTGTAVV | | |
| | | RSSRVPTGAT | | |
| | | TQDAEVDDAK YGFVASGTLN | | |
| | | PQKARVLLQL ALTQTKDPQQ | | |
| | | IQQIFNQY | | |
| 68 | E. coli L- | MQKKSIYVAY TGGTIGMQRS | | E. coli L- |
| | asparaginase 1 | EQGYIPVSGH LQRQLALMPE | | asparaginase 1 |
| | NCBI | FHRPEMPDFT IHEYTPLMDS | | NCBI |
| | Accession | SDMTPEDWQH IAEDIKAHYD | | Accession |
| | No. | DYDGFVILHG TDTMAYTASA | | No. |
| | NP_416281.1 | LSFMLENLGK PVIVTGSQIP | | NP_416281.1 |
| | | LAELRSDGQI NLLNALYVAA | | |
| | | NYPINEVTLF FNNRLYRGNR | | |
| | | TTKAHADGFD AFASPNLPPL | | |
| | | LEAGIHIRRL NTPPAPHGEG | | |
| | | ELIVHPITPQ PIGVVTIYPG | | |
| | | ISADVVRNFL RQPVKALILR | | |
| | | SYGVGNAPQN KAFLQELQEA | | |
| | | SDRGIVVVNL TQCMSGKVNM | | |
| | | GGYATGNALA | | |
| | | HAGVIGGADM | | |
| | | TVEATLTKLH YLLSQELDTE | | |
| | | TIRKAMSQNL RGELTPDD | | |
| 69 | Staphylococcus | MKHLLVIHTG GTISMSQDQS | | Staphylococcus |
| | aureus L- | NKVVTNDINP ISMHQDVINQ | | aureus L- |
| | asparaginase | YAQIDELNPF NVPSPHMTIQ | | asparaginase |
| | NCBI | HVKQLKDIIL EAVTNKYYDG | | NCBI |
| | Accession | FVITHGTDTL EETAFLLDLI | | Accession |
| | No. | LGIEQPVVIT GAMRSSNEIG | | No. |
| | YP_500016.1 | SDGLYNYISA IRVASDEKAR | | YP_500016.1 |
| | | HKGVMVVFND EIHTARNVTK | | |
| | | THTSNTNTFQ SPNHGPLGVL | | |
| | | TKDRVQFHHM | | |
| | | PYRQQALENV NDKLNVPLVK | | |
| | | AYMGMPGDIF SFYSREGIDG | | |
| | | MVIEALGQGN | | |
| | | IPPSALEGIQ QLVSLNIPIV | | |
| | | LVSRSFNGIV SPTYAYDGGG | | |
| | | YQLAQQGFIF SNGLNGPKAR | | |
| | | LKLLVALSNN LDKAEIKSYF | | |
| | | EL | | |
| 5 | Erwinia | MFNALFVVVF VCFSSLANAA | Kₘ = 0.085-0.098 mM | Kₘ = 3.0-6.8 mM |
| | carotovora | ENLPNIVILA TGGTIAGSAA | k_{cat} = 524-1033 s⁻¹ | k_{cat} = 2.9-7.6 s⁻¹ |
| | L- | ANTQTTGYKA GALGVETLIQ | | |
| | asparaginase | AVPELKTLAN IKGEQVASIG | | |
| | UniProt | SENMTSDVLL TLSKRVNELL | | |
| | I1SBD9 | ARSDVDGVVI THGTDTLDES | | |
| | | PYFLNLTVKS DKPVVFVAAM | | |
| | | RPATAISADG PMNLYGAVKV | | |
| | | AADKNSRGRG VLVVLNDRIG | | |
| | | SARFISKTNA STLDTFKAPE | | |
| | | EGYLGVIIGD KIYYQTRLDK | | |
| | | VHTTRSVFDV TNVDKLPAVD | | |
| | | IIYGYQDDPE YMYDASIKHG | | |
| | | VKGIVYAGMG | | |
| | | AGSVSKRGDA GIRKAESKGI | | |
| | | VVVRSSRTGS | | |
| | | GIVPPDAGQP GLVADSLSPA | | |
| | | KSRILLMLAL TKTTNPAVIQ | | |
| | | DYFHAY | | |
| 6 | Glutaminase- | KEVENQQKLA NVVILATGGT | Kₘ = 0.0046 mM | Kₘ = 0.0044 mM |
| | asparaginase | IAGAGASAAN SATYQAAKVG | k_{cat} = 93.1 s⁻¹ | k_{cat} = 93.1 s⁻¹ |
| | UniProt | VDKLIAGVPE LADLANVRGE | | |
| | P10182 | QVMQIASESI TNDDLLKLGK | | |
| | | RVAELADSND VDGIVITHGT | | |
| | | DTLEETAYFL DLTLNTDKPI | | |
| | | VVVGSMRPGT | | |
| | | AMSADGMLNL | | |
| | | YNAVAVASNK | | |
| | | DSRGKGVLVT MNDEIQSGRD | | |
| | | VSKSINIKTE AFKSAWGPLG | | |
| | | MVVEGKSYWF | | |
| | | RLPAKRHTVN SEFDIKQISS | | |
| | | LPQVDIAYSY GNVTDTAYKA | | |
| | | LAQNGAKALI HAGTGNGSVS | | |
| | | SRLTPALQTL RKTGTQIIRS | | |
| | | SHVNQGGFVL RNAEQPDDKN | | |
| | | DWVVAHDLNP EKARILVELA | | |
| | | MVKTQDSKEL QRIFWEY | | |
| 70 | Pseudomonas | KEVENQQKLA NVVILATGGT | Kₘ = 0.0046 mM | Kₘ = 0.0044 mM |
| | 7A | IAGAGASAAN SATYQAAKVG | k_{cat} = 93.1 s⁻¹ | k_{cat} = 93.1 s⁻¹ |
| | glutaminase- | VDKLIAGVPE LADLANVRGE | | |
| | asparaginase | QVMQIASESI TNDDLLKLGK | | |
| | | RVAELADSND VDGIVITHGT | | |
| | | DTLEETAYFL NLVEKTDKPI | | |
| | | VVVGSMRPGT | | |
| | | AMSADGMLNL | | |
| | | YNAVAVASNK | | |
| | | DSRGKGVLVT MNDEIQSGRD | | |
| | | VSKSINIKTE AFKSAWGPLG | | |
| | | MVVEGKSYWF | | |
| | | RLPAKRHTVN SEFDIKQISS | | |
| | | LPQVDIAYSY GNVTDTAYKA | | |
| | | LAQNGAKALI HAGTGNGSVS | | |
| | | SRVVPALQEL RKNGVQIIRS | | |
| | | SHVNQGGFVL RNAEQPDDKN | | |
| | | DWVVAHDLNP | | |
| | | QKARILAMVA MTKTQDSKEL | | |
| | | QRIFWEY | | |
| 7 | Acinetobacter | KNNVVIVATG GTIAGAGASS | Kₘ = 0.04-0.07 mM | Kₘ = 0.04-0.07 mM |
| | glutaminasificans | TNSATYSAAK VPVDALIKAV | k_{cat} = 60 s⁻¹ | k_{cat} = 60 s⁻¹ |
| | glutaminase- | PQVNDLANIT GIQALQ VASE | | |
| | asparaginase | SITDKELLSL ARQVNDLVKK | | |
| | UniProt | PSVNGVVITH GTDTMEETAF | | |
| | P10172 | FLNLVVHTDK PIVLVGSMRP | | |
| | | STALSADGPL NLYSAVALAS | | |
| | | SNEAKNKGVM VLMNDSIFAA | | |
| | | RDVTKGINIH THAFVSQWGA | | |
| | | LGTLVEGKPY WFRSSVKKHT | | |
| | | NNSEFNIEKI QGDALPGVQI | | |
| | | VYGSDNMMPD | | |
| | | AYQAFAKAGV KAIIHAGTGN | | |
| | | GSMANYLVPE VRKLHDEQGL | | |
| | | QIVRSSRVAQ GFVLRNAEQP | | |
| | | DDKYGWIAAH | | |
| | | DLNPQKARLL MALALTKTND | | |
| | | AKEIQNMFWN Y | | |
| 8 | Wolinella | MAKPQVTILA TGGTIAGSGE | Kₘ = 0.0478 mM | Kₘ = n/a |
| | succinogenes | SSVKSSYSAG AVTVDKLLAA | k_{cat} = 166.6 s⁻¹ | k_{cat} = n/a |
| | L- | VPAINDLATI KGEQISSIGS | | |
| | asparaginase | QEMTGKVWLK | | |
| | UniProt | LAKRVNELLA QKETEAVIIT | | |
| | P50286 | HGTDTMEETA FFLNLTVKSQ | | |
| | | KPVVLVGAMR SGSSMSADGP | | |
| | | MNLYNAVNVA | | |
| | | INKASTNKGV VIVMNDEIHA | | |
| | | AREATKLNTT AVNAFASPNT | | |
| | | GKIGTVYYGK VEYFTQSVRP | | |
| | | HTLASEFDIS KIEELPRVDI | | |
| | | LYAHPDDTDV | | |
| | | LVNAALQAGA KGIIHAGMGN | | |
| | | GNPFPLTQNA LEKAAKSGVV | | |
| | | VARSSRVGSG STTQEAEVDD | | |
| | | KKLGFVATES | | |
| | | LNPQKARVLL MLALTKTSDR | | |
| | | EAIQKIFSTY | | |

TABLE 3
Exemplary amino acid sequences of amino acid
degradative enzymes that inhibit tumor growth.

| SEQ | Sequence | |
|---|---|---|
| ID | name and | |
| NO: | source | Amino acid sequence |
| 9 | Homo sapiens | MMSGEPLHVK TPIRDSMALS KMAGTSVYLK |
| | L-serine | MDSAQPSGSF KIRGIGHFCK RWAKQGCAHF |
| | dehydratase | VCSSAGNAGM AAAYAARQLG VPATIVVPST |
| | UniProt | TPALTIERLK NEGATVKVVG ELLDEAFELA |
| | P20132 | KALAKNNPGW VYIPPFDDPL IWEGHASIVK |
| | | ELKETLWEKP GAIALSVGGG GLLCGVVQGL |
| | | QEVGWGDVPV IAMETFGAHS |
| | | FHAATTAGKL VSLPKITSVA KALGVKTVGA |
| | | QALKLFQEHP IFSEVISDQE AVAAIEKFVD |
| | | DEKILVEPAC GAALAAVYSH VIQKLQLEGN |
| | | LRTPLPSLVV IVCGGSNISL AQLRALKEQL GMTNRLPK |
| 71 | Escherichia | MISVFDIFKI GIGPSSSHTV GPMKAGKQFT |
| | coli 0157:H7 | DDLIARNLLK DVTRVVVDVY GSLSLTGKGH |
| | str. Sakai | HTDIAIIMGL AGNLPDTVDI DSIPGFIQDV |
| | L-serine | NTHGRLMLAN GQHEVEFPVD QCMNFHADNL |
| | dehydratase | SLHENGMRIT ALAGDKVVYS QTYYSIGGGF |
| | NCBI | IVDEEHFGQQ DSAPVEVPYP YSSAADLQKH |
| | NP_311684.1 | CQETGLSLSG LMMKNELALH SKEELEQHLA |
| | | NVWEVMCGGI ERGISTEGVL PGKLRVPRRA |
| | | AALRRMLVSQ DKTTTDPMAV VDWINMFALA |
| | | VNEENAAGGR VVTAPTNGAC GIIPAVLAYY |
| | | DKFIREVNAN SLARYLLVAS AIGSLYKMNA |
| | | SISGAEVGCQ GEVGVACSMA AAGLAELLGA |
| | | SPAQVCIAAE IAMEHNLGLT CDPVAGQVQV |
| | | PCIERNAIAA VKAVNAARMA LRRTSEPRVC |
| | | LDKVIETMYE TGKDMNAKYR ETSRGGLAMK IVACD |
| 72 | Pseudomonas | MSLSVFDLFK IGIGPSSSHT VGPMRAAARF |
| | aeruginosa | AEGLRREGLL EATASIKVEL YGSLGATGKG |
| | L-serine | HGSDKAVLLG LEGEQPDTVD TAAIPARLDA |
| | dehydratase | IRSSGELRLL GERPIRFVEK EHLALIRKPL |
| | NCBI | AYHPNGMIFR AFDAAGLQVR SREYYSVGGG |
| | NP_251133.1 | FVVDDEAAGL DRIVEDRTPL AFPFKTARQL |
| | | LDHCAREGLS ISQLMAENEK AWRPAEETRT |
| | | GLLRIWQVMQ DCVEAGCRNE GIMPGGLKVR |
| | | RRAAALHRQL CQRPEAGLRD ALSVLDWVNL |
| | | YALAVNEENA SGGRVVTAPT NGAAGIIPAV |
| | | LHYYARFIPG ADDDGVVRFL LTAAAIGILY |
| | | KENASISGAE VGCQGEVGVA CSMAAGALCE |
| | | VLGGSVQQVE NAAEIGMEHN LGLTCDPVGG |
| | | LVQVPCIERN AMASVKAINA ARMALRGDGQ |
| | | HFISLDKVIR TMRQTGADMK SKYKETARGG LAVNIIEC |
| 10 | Homo sapiens | MTMPVNGAHK DADLWSSHDK MLAQPLKDSD |
| | Serine | VEVYNIIKKE SNRQRVGLEL IASENFASRA |
| | hydroxymethyltransferase | VLEALGSCLN NKYSEGYPGQ RYYGGTEFID |
| | UniProt | ELETLCQKRA LQAYKLDPQC WGVNVQPYSG |
| | P34896 | SPANFAVYTA LVEPHGRIMG LDLPDGGHLT |
| | | HGFMTDKKKI SATSIFFESM PYKVNPDTGY |
| | | INTYDQLEENA RLFHPKLIIA |
| | | GTSCYSRNLE YARLRKIADE NGAYLMADMA |
| | | HISGLVAAGV VPSPFEHCHV VTTTTHKTLR |
| | | GCRAGMIFYR KGVKSVDPKT GKEILYNLES |
| | | LINSAVFPGL QGGPHNHAIA GVAVALKQAM |
| | | TLEFKVYQHQ VVANCRALSE ALTELGYKIV |
| | | TGGSDNHLIL VDLRSKGTDG GRAEKVLEAC |
| | | SIACNKNTCP GDRSALRPSG |
| | | LRLGTPALTS RGLLEKDFQK VAHFIHRGIE |
| | | LTLQIQSDTG VRATLKEFKE RLAGDKYQAA |
| | | VQALREEVES FASLFPLPGL |
| | | PDF |
| 73 | Escherichia | MLKREMNIAD YDAELWQAME QEKVRQEEHI |
| | coli str. K-12 | ELIASENYTS PRVMQAQGSQ LTNKYAEGYP |
| | substr. | GKRYYGGCEY VDIVEQLAID RAKELFGADY |
| | MG1655 | ANVQPHSGSQ ANFAVYTALL EPGDTVLGMN |
| | Serine | LAHGGHLTHG SPVNFSGKLY NIVPYGIDAT |
| | hydroxymethyltransferase | GHIDYADLEK QAKEHKPKMI IGGFSAYSGV |
| | NCBI | VDWAKMREIA DSIGAYLFVD MAHVAGLVAA |
| | NP_417046.1 | GVYPNPVPHA HVVTTTTHKT LAGPRGGLIL |
| | | AKGGSEELYK KLNSAVFPGG QGGPLMHVIA |
| | | GKAVALKEAM EPEFKTYQQQ VAKNAKAMVE |
| | | VFLERGYKVV SGGTDNHLFL VDLVDKNLTG |
| | | KEADAALGRA NITVNKNSVP NDPKSPFVTS |
| | | GIRVGTPAIT RRGFKEAEAK ELAGWMCDVL |
| | | DSINTDEAVIE RIKGKVLDIC ARYPVYA |
| 74 | Staphylococcus | MSYITKQDKV IAEAIEREFQ RQNSNIELIA |
| | aureus | SENFVSEAVM EAQGSVLTNK YAEGYPGRRY |
| | Serine | YGGCEFVDVT ESIAIDRAKA LFGAEHVNVQ |
| | hydroxymethyltransferase | PHSGSQANMA VYLVALEMGD TVLGMNLSHG |
| | NCBI | GHLTHGAPVN FSGKFYNFVE YGVDKDTERI |
| | YP_500830.1 | NYDEVRKLAL EHKPKLIVAG ASAYSRTIDF |
| | | KKFKEIADEV NAKLMVDMAH IAGLVAAGLH |
| | | PNPVEYADFV TTTTHKTLRG PRGGMILCKE |
| | | EYKKDIDKTI FPGIQGGPLE HVIAAKAVAF |
| | | GEALENNFKT YQQQVVKNAK VLAEALINEG |
| | | FRIVSGGTDN HLVAVDVKGS IGLTGKEAEE |
| | | TLDSVGITCN KNTIPFDQEK PFVTSGIRLG |
| | | TPAATTRGFD EKAFEEVAKI ISLALKNSKD |
| | | EEKLQQAKER VAKLTAEYPL YQ |
| 11 | Homo sapiens | MSAKSRTIGI IGAPFSKGQP RGGVEEGPTV |
| | Arginase-1 | LRKAGLLEKL KEQECDVKDY GDLPFADIPN |
| | UniProt | DSPFQIVKNP RSVGKASEQL AGKVAEVKKN |
| | P05089 | GRISLVLGGD HSLAIGSISG HARVHPDLGV |
| | | IWVDAHTDIN TPLTTTSGNL HGQPVSFLLK |
| | | ELKGKIPDVP GFSWVTPCIS AKDIVYIGLR |
| | | DVDPGEHYIL KTLGIKYFSM |
| | | TEVDRLGIGK VMEETLSYLL GRKKRPIHLS |
| | | FDVDGLDPSF TPATGTPVVG GLTYREGLYI |
| | | TEEIYKTGLL SGLDIMEVNP SLGKTPEEVT |
| | | RTVNTAVAIT LACFGLAREG NHKPIDYLNP |
| | | PK |
| 75 | Mus musculus | MSSKPKSLEIIGAPFSKGQPRGGVEKGPAALRKAGLLEK |
| | Arginase-1 | LKETEYDVRDHGDLAFVDVPNDSSFQIVKNP |
| | NCBI | RSVGKANEELAGVVAEVQKNGRVSVVLGGDHSLAVGS |
| | NP_031508.1 | ISGHARVHPDLCVIWVDAHTDINTPLTTSSGNL |
| | | HGQPVSFLLKELKGKFPDVPGFSWVTPCISAKDIVYIGLR |
| | | DVDPGEHYIIKTLGIKYFSMTEVDKLGIGK |
| | | VMEETFSYLLGRKKRPIHLSFDVDGLDPAFTPATGTPVL |
| | | GGLSYREGLYITEEIYKTGLLSGLDIMEVNP |
| | | TLGKTAEEVKSTVNTAVALTLACFGTQREGNHKPGTDY |
| | | LKPPK |
| 76 | Danio rerio | MMKMKSLSGSRAALHIFRRHLHHQRYSVGIIGAPFSKG |
| | Arginase-1 | QQKDGVQEGADLIRAAGLVQKLKGQGCVVKDY |
| | NCBI | GNVTFENLPNDESIGRLKTPRAVGRANELLSGAVQKIKS |
| | NP_001038662.1 | DGNTCVMLGGDHSLAIGSISGHAAYRHELSV |
| | | LWVDAHADINTPLTTPTGNIHGQPMSYLIHELHSKMPKL |
| | | PNFSWLKPCIAAQDVVYIGLRDVDPEEHYIL |
| | | KYLGIKTFSMTEVDRLGIAKVMEQTCDHMFSKVKKPIH |
| | | LSFDIDALDPSVSPATGTPVAGGLTYREGIYI |
| | | TEHICQTGLLSAVDMVEVNPKLGRTADEIKSTVNAAVD |
| | | LLLGCFGRIREGSHDPDYKMPNP |
| 77 | Xenopus | MSSQAKTSVGVIGAPFSKGQPRRGVEEGPKYLRDAGVIE |
| | tropicalis | KLRELGNDVRDYGDLDFPDVPNDITFNNVKN |
| | Arginase-1 | PRTVGKATEKLANAVTAVKKADRTCLVIGGDHSLAVGT |
| | NCBI | IAGHAAVHRDLCVVWVDAHADINTPSTSPSGN |
| | NP_001006714.1 | LHGQPLSFLMKELKSKMPDVPGFEWVKPCLSAKDIVYI |
| | | GLRDVDPGEHYILKTLGIKYYSMSEVDYLKID |
| | | KVMEETIEYLVGKQKRPIHLSFDIDGLDPSIAPATGTAVP |
| | | GGLTYREGMYITEKLCRTGLLSAVDIMEVN |
| | | PSRGETKRDVELTVNTALDMTLSCFGKAREGYHTSTMT |
| | | LPDII |
| 12 | Pseudomonas | MSTEKTKLGV HSEAGKLRKV MVCSPGLAHQ |
| | aeruginosa | RLTPSNCDEL LFDDVIWVNQ AKRDHFDFVT |
| | Arginine | KMRERGIDVL EMHNLLTETI QNPEALKWIL |
| | deiminase | DRKITADSVG LGLTSELRSW LESLEPRKLA |
| | UniProt | EYLIGGVAAD DLPASEGANI LKMYREYLGH |
| | P13981 | SSFLLPPLPN TQFTRDTTCW IYGGVTLNPM |
| | | YWPARRQETL LTTAIYKFHP |
| | | EFANAEFEIW YGDPDKDHGS STLEGGDVMP |
| | | IGNGVVLIGM GERSSRQAIG QVAQSLFAKG |
| | | AAERVIVAGL PKSRAAMHLD TVFSFCDRDL |
| | | VTVFPEVVKE IVPFSLRPDP SSPYGMNIRR |
| | | EEKTFLEVVA ESLGLKKLRV VETGGNSFAA |
| | | EREQWDDGNN VVCLEPGVVV GYDRNTYTNT |
| | | LLRKAGVEVI TISASELGRG |
| | | RGGGHCMTCP IVRDPIDY |
| 78 | Escherichia | MEKHYVGSEIGQLRSVMLHRPNLSLKRLTPSNCQELLFD |
| | coli | DVLSVERAGEEHDIFANTLRDQGVEVLLLTD |
| | Arginine | LLTQTLDIKEAKTWLLETQISDYRLGPTFAGDVRSWLAD |
| | deiminase | MPHRELARRLSGGLTYGEIPAAINNMVVDTH |
| | NCBI | TSNDFIMKPLPNHLFTRDTSCWIYNGVSINPMAKPARQR |
| | YP_003937657.1 | ETNNLRAIYRWHPAFADGDFIKYFGDENIYY |
| | | DHATLEGGDVLVIGRGAVLIGMSERTTPQGVEFLANSLF |
| | | KHRQAERVIAVELPKHRSCMHLDTVMTHIDV |
| | | DTFSVYPEVVRKDAQCWTLTSNGRDGLQRTQETDLLH |
| | | AIEKALGIDQVRLITTGGDAFEAEREQWNDANN |
| | | VLTIRPGVVIGYERNVWTNEKYDKAGITVLPIPGDELGR |
| | | GRGGARCMSCPLERDGI |
| 79 | Staphylococcus | MTDGPIKVNSEIGALKTVLLKRPGKELENLVPDYLDGLL |
| | aureus | FDDIPYLEVAQKEHDHFAQVLREEGVEVLYL |
| | Arginine | EKLAAESIENPQVRSEFIDDVLAESKKTILGHEEEIKALFA |
| | deiminase | TLSNQELVDKIMSGVRKEEINPKCTHLVE |
| | NCBI | YMDDKYPFYLDPMPNLYFTRDPQASIGHGITINRMFWR |
| | YP_501419.1 | ARRRESIFIQYIVKHHPRFKDANIPIWLDRDC |
| | | PFNIEGGDELVLSKDVLAIGVSERTSAQAIEKLARRIFEN |
| | | PQATFKKVVAIEIPTSRTFMHLDTVFTMID |
| | | YDKFTMHSAILKAEGNMNIFIIEYDDVNKDIAIKQSSHLK |
| | | DTLEDVLGIDDIQFIPTGNGDVIDGAREQW |
| | | NDGSNTLCIRPGVVVTYDRNYVSNDLLRQKGIKVIEISG |
| | | SELVRGRGGPRCMSQPLFREDI |
| 13 | Pseudomonas | MHGSNKLPGF ATRAIHHGYD PQDHGGALVP |
| | putida | PVYQTATFTF PTVEYGAACF AGEQAGHFYS |
| | L-methionine | RISNPTLNLL EARMASLEGG EAGLALASGM |
| | gamma-lyase | GAITSTLWTL LRPGDEVLLG NTLYGCTFAF |
| | UniProt | LHHGIGEFGV KLRHVDMADL QALEAAMTPA |
| | P13254 | TRVIYFESPA NPNMHMADIA GVAKIARKHG |
| | | ATVVVDNTYC TPYLQRPLEL |
| | | GADLVVHSAT KYLSGHGDIT AGIVVGSQAL |
| | | VDRIRLQGLK DMTGAVLSPH DAALLMRGIK |
| | | TLNLRMDRHC ANAQVLAEFL ARQPQVELIH |
| | | YPGLASFPQY TLARQQMSQP GGMIAFELKG |
| | | GIGAGRRFMN ALQLFSRAVS LGDAESLAQH |
| | | PASMTHSSYT PEERAHYGIS EGLVRLSVGL |
| | | EDIDDLLADV QQALKASA |
| 80 | Erwinia | MPSSHSKKTHIGQRELQPETQMLNYGYDPALSEGAVKP |
| | amylovora | PVFLTSTFIFNSAEEGRDFFDYVSGRREPPAG |
| | L-methionine | EGNGLVYSRFNHPNSEIVEDRLAIYERSESAALFSSGMS |
| | gamma-lyase | AIATTLLTFVRPGDAILHSQPLYGGSETLLS |
| | NCBI | KTFGNLGVAAIGFADGIDEALVQAAADKALAQGRVSAI |
| | WP_004159559.1 | LIESPANPTNSLVDIALIKRVADRIEQQQQHR |
| | | PVIACDNTLLGPVFSRPLEHGADISLYSLTKYVGGHSDLI |
| | | AGAAMGNRALIRQVKALRSAIGTQLDAHSS |
| | | WMIGRSLETLALRMDRANDNAAAVAEFLRSHSLVEQIH |
| | | YLPFIDPHSAAGKVYSDQCSGAGSTFSFDIRG |
| | | GQDAAFRFLNGLQLFKLAVSLGGTESLASHPASTTHSGV |
| | | DLAVRERMGIKASTLRLSIGIENKDDLIEDL |
| | | RLSLDR |
| 81 | Clostridium | MENIKKMGFATKAIHGGHIGDKQFGSLATPIYQTSTFIFD |
| | botulinum A | SAEQGGRRFAGEESGYIYSRLGNPTSTEVE |
| | L-methionine | NKLALLECGEAAVVAASGMGAIAASLWSALKSGDHVV |
| | gamma-lyase | ASDTLYGCTFALLNHGLTRYGVEVTFVDVSNLD |
| | NCBI | EVRNALKANTKVVYLETPANPTLKVTDIKQISNMVHEN |
| | YP_001252577.1 | NKECLVFVDNTFCTPYIQRPLQLGADVVVHSA |
| | | TKYLNGHGDVIAGFAVGKEEFINQVKLFGIKDMTGSVIG |
| | | PFEAFLIIRGMKTLQLRMEKHCKNAMEVAKF |
| | | LESHPAVKKVYYPGLESFEYYELAKKQMSLPGAMISFEL |
| | | KGGVEEGKVVMNNVKLATLAVSLGDAETLIQ |
| | | HPASMTHSPYTAEERKEAGISDGLVRLSVGLEDVDDIIS |
| | | DLKQALDLIVK |
| 14 | Homo sapiens | MAPLALHLLV LVPILLSLVA SQDWKAERSQ |
| | L-amino-acid | DPFEKCMQDP DYEQLLKVVT WGLNRTLKPQ |
| | oxidase | RVIVVGAGVA GLVAAKVLSD AGHKVTILEA |
| | UniProt | DNRIGGRIFT YRDQNTGWIG ELGAMRMPSS |
| | Q96RQ9 | HRILHKLCQG LGLNLTKFTQ YDKNTWTEVH |
| | | EVKLRNYVVE KVPEKLGYAL RPQEKGHSPE |
| | | DIYQMALNQA LKDLKALGCR |
| | | KAMKKFERHT LLEYLLGEGN LSRPAVQLLG |
| | | DVMSEDGFFY LSFAEALRAH SCLSDRLQYS |
| | | RIVGGWDLLP RALLSSLSGL VLLNAPVVAM |
| | | TQGPHDVHVQ IETSPPARNL KVLKADVVLL |
| | | TASGPAVKRI TFSPPLPRHM QEALRRLHYV |
| | | PATKVFLSFR RPFWREEHIE GGHSNTDRPS |
| | | RMIFYPPPRE GALLLASYTW SDAAAAFAGL |
| | | SREEALRLAL DDVAALHGPV VRQLWDGTGV |
| | | VKRWAEDQHS QGGFVVQPPA LWQTEKDDWT |
| | | VPYGRIYFAG EHTAYPHGWV ETAVKSALRA |
| | | AIKINSRKGP ASDTASPEGH ASDMEGQGHV |
| | | HGVASSPSHD LAKEEGSHPP VQGQLSLQNT THTRTSH |
| 82 | Mus musculus | MSFRTMAKKSGILVWGILLCVSSCLALYENLVKCFQDP |
| | L-amino-acid | DYEAFLLIAQNGLHTSPLSKRVVVVGAGMAGL |
| | oxidase | VAAKTLQDAGHEVTILEASNHIGGRVVTLRNKEEGWYL |
| | NCBI | ELGPMRIPESHKLIHTYVQKLGLKLNKFHQYD |
| | NP_598653.3 | SNTWYLLNGQRYRASEVMANPGILGYPLRPSEKNKTVT |
| | | DLFYQAITKIKPHRKTSNCSQLLSLYDSYSTK |
| | | AYLMKEGTLSKGAIEMIGDIMNENAGYYKSLLESLRIAS |
| | | IFSKSDQFSEITGGFDQLPNGLSASLKPGTI |
| | | RLGSKVERVVRDGPKVKVMYRTDGPTSALHKLTADYA |
| | | IITASAKATRLITFQPPLSREKTHALRSVHYTS |
| | | ATKVVLVCNERFWEQDGIRGGYSITDRPSRFIYYPSHSLP |
| | | GGKGVLLASFTVGDDSSFFAALKPNQVVDV |
| | | VLDDLAAVHRIPKEELKRMCPKSAIKHWSLDPLTIGAFT |
| | | EFTPYQFVDYSKQLSQPEGRIYFAGEHTCLP |
| | | HSWIDTAIKSGIRASCNIQAAVDKEATRGHTAL |
| 83 | Rattus | MSFRTMAKKSGILIWGILLSISSCLASFEDIFKCFQDPDYE |
| | norvegicus | ALLLIAQNGLHTSPSSKRIVVVGAGMAGL |
| | L-amino-acid | VAAKLLQDAGHEVTILEASNHIGGRVVTLRNKEEGWHF |
| | oxidase | ELGPMRIPESHRIIHTYIQKFGLKLNNFTQHD |
| | NCBI | NNTWYLLRGHRYRASEVKANPEILGYPLRPSEKNKTAE |
| | NP_001100152.1 | DLFYQAITKVKASNCSQLLSLYDSYSTKAYLL |
| | | KEGMLSRGAVEMIGDMMNENAGFYRSLLESLRIANIFT |
| | | KNDQFTEITGGFDQLPNSLNDSLKPGTIHLGS |
| | | KVERVVGNESKVEVLYRTDGPTSALYNLTADYVIISASA |
| | | KATRLIAFQPPLSPEKIRALRSVHYNSATKV |
| | | IFVCNERFWEKDGIHGGYSITDRPSRFIYYPSYSRPSSKGI |
| | | LLASFTMDDDSFFFTALKPNQVVDIILDD |
| | | LAAVHLIPKEELKRMCPKSEVKHWSLDPFTIGSYAEFTP |
| | | YQFLDDLKQLSQTEGRIYFAGEHTSLPHAWI |
| | | ETAIKSGIRAAKNIQDTVDKEATQGQVAL |
| 15 | Escherichia | MAKHLFTSES VSEGHPDKIA DQISDAVLDA |
| | coli | ILEQDPKARV ACETYVKTGM VLVGGEITTS |
| | S- | AWVDIEEITR NTVREIGYVH SDMGFDANSC |
| | adenosylmethionine | AVLSAIGKQS PDINQGVDRA DPLEQGAGDQ |
| | synthase | GLMFGYATNE TDVLMPAPIT YAHRLVQRQA |
| | UniProt | EVRKNGTLPW LRPDAKSQVT FQYDDGKIVG |
| | P0A817 | IDAVVLSTQH SEEIDQKSLQ |
| | | EAVMEEIIKP ILPAEWLTSA TKFFINPTGR |
| | | FVIGGPMGDC GLTGRKIIVD TYGGMARHGG |
| | | GAFSGKDPSK VDRSAAYAAR YVAKNIVAAG |
| | | LADRCEIQVS YAIGVAEPTS IMVETFGTEK |
| | | VPSEQLTLLV REFFDLRPYG LIQMLDLLHP |
| | | IYKETAAYGH FGREHFPWEK TDKAQLLRDA AGLK |
| 84 | Pseudomonas | MSEYSLFTSESVSEGHPDKIADQISDAVLDAIIAQDKYAR |
| | entomophila | VACETLVKTGVAIIAGEVTTSAWVDLEELV |
| | S- | RKVIIDIGYNSSDVGFDGATCAVMNIIGKQSVDIAQGVD |
| | adenosylmethionine | RSKPEDQGAGDQGLMFGYASNETDVLMPAPI |
| | synthase | CFSHRLVERQAEARKSGLLPWLRPDAKSQVTCRYENGR |
| | NCBI | VVGIDAVVLSTQHNPEVSQKDLQEAVMELIVK |
| | WP_011536020.1 | HTLPAELLHKGTQYHINPTGNFIIGGPVGDCGLTGRKIIV |
| | | DSYGGMARHGGGAFSGKDPSKVDRSAAYAG |
| | | RYVAKNIVAAGLAERCEIQVSYAIGVAQPTSISINTFGTG |
| | | KVSDDKIVQLVRECFDLRPYAITKMLDLLH |
| | | PMYQETAAYGHFGRTPQQKTVGDDTFTTFTWERTDRA |
| | | QALRDAAGL |
| 85 | Staphylococcus | MTYNKRLFTSESVTEGHPDKIADQVSDAILDEILKDDPN |
| | saprophyticus | ARVACETTVTTGMALISGEISTTTYVDIPKV |
| | S- | VRETIKEIGYTRAKFGYDSQTMAVLTAIDEQSPDIAQGV |
| | adenosylmethionine | DTALEYRDEASEAEIEATGAGDQGLMFGYAT |
| | synthase | NETDTYMPLPIFLSHQLAKRLSDVRKDEILKYLRPDGKV |
| | NCBI | QVTVEYDEQDKPVRIDTIVLSTQHAEDIELD |
| | WP_011302830.1 | QIKDDIKTHVIYPTVPESLLDEQTKFYINPTGRFVIGGPQ |
| | | GDAGLTGRKIIVDTYGGYARHGGGCFSGKD |
| | | PTKVDRSAAYAARYVAKNIVAAQLAEKCEVQLAYAIG |
| | | VAEPVSISIDTFGTGKVSEYELVEAVRKHFDLR |
| | | PAGIIKMLDLKHPIYKQTAAYGHFGRTDVLLPWEKLDK |
| | | VNLLKDSVKA |
| 16 | Homo sapiens | MQEKDASSQG FLPHFQHFAT QAIHVGQDPE |
| | Engineered | QWTSRAVVPP ISLSTTFKQG APGQHSGFNY |
| | cystathionine | SRSGNPTRNC LEKAVAALDG AKYCLAFASG |
| | gamma-lyase | LAATVTITHL LKAGDQIICM DDVYGGTNLY |
| | UniProt | FRQVASEFGL KISFVDCSKI KLLEAAITPE TKLVWIETPT |
| | P32929 | NPTQKVIDIE GCAHIVHKHG DIILVVDNTF |
| | | MSPYFQRPLA |
| | | LGADISMYSA TKYMNGHSDV VMGLVSVNCE |
| | | SLHNRLRFLQ NSLGAVPSPI DCYLCNRGLK |
| | | TLHVRMEKHF KNGMAVAQFL ESNPWVEKVI |
| | | YPGLPSHPQH ELVKRQCTGC TGMVTFYIKG |
| | | TLQHAEIFLK NLKLFTLAVS LGGFESLAEL |
| | | PAIMTHASVL KNDRDVLGIS DTLIRLSVGL |
| | | EDEEDLLEDL DQALKAAHPP |
| | | SGSHS |
| 86 | Mus musculus | MQKDASLSGFLPSFQHFATQAIHVGQEPEQWNSRAVVL |
| | Cystathionine | PISLATTFKQDFPGQSSGFEYSRSGNPTRNCL |
| | gamma-lyase | EKAVAALDGAKHSLAFASGLAATITITHLLKAGDEIICM |
| | NCBI | DEVYGGTNRYFRRVASEFGLKISFVDCSKTK |
| | NP_666065.1 | LLEAAITPQTKLVWIETPTNPTLKLADIGACAQIVHKRG |
| | | DIILVVDNTFMSAYFQRPLALGADICMCSAT |
| | | KYMNGHSDVVMGLVSVNSDDLNSRLRFLQNSLGAVPS |
| | | PFDCYLCCRGLKTLQVRMEKHFKNGMAVARFLE |
| | | TNPRVEKVVYPGLPSHPQHELAKRQCSGCPGMVSFYIK |
| | | GALQHAKAFLKNLKLFTLAESLGGYESLAELP |
| | | AIMTHASVPEKDRATLGINDTLIRLSVGLEDEQDLLEDL |
| | | DRALKAAHP |
| 87 | Saccharomyces | MTLQESDKFATKAIHAGEHVDVHGSVIEPISLSTTFKQSS |
| | cerevisiae | PANPIGTYEYSRSQNPNRENLERAVAALEN |
| | Cystathionine | AQYGLAFSSGSATTATILQSLPQGSHAVSIGDVYGGTHR |
| | gamma-lyase | YFTKVANAHGVETSFTNDLLNDLPQLIKENT |
| | NCBI | KLVWIETPTNPTLKVTDIQKVADLIKKHAAGQDVILVVD |
| | NP_009390.1 | NTFLSPYISNPLNFGADIVVHSATKYINGHS |
| | | DVVLGVLATNNKPLYERLQFLQNAIGAIPSPFDAWLTHR |
| | | GLKTLHLRVRQAALSANKIAEFLAADKENVV |
| | | AVNYPGLKTHPNYDVVLKQHRDALGGGMISFRIKGGAE |
| | | AASKFASSTRLFTLAESLGGIESLLEVPAVMT |
| | | HGGIPKEAREASGVFDDLVRISVGIEDTDDLLEDIKQALK |
| | | QATN |
| 17 | Pseudomonas | MKQIAFIGLG HMGAPMATNL LKAGYLLNVF |
| | aeruginosa | DLVQSAVDGL VAAGASAARS ARDAVQGADV |
| | NAD- | VISMLPASQH VEGLYLDDDG LLAHIAPGTL |
| | dependent L- | VLECSTIAPT SARKIHAAAR ERGLAMLDAP |
| | serine | VSGGTAGAAA GTLTFMVGGD AEALEKARPL |
| | dehydrogenase | FEAMGRNIFH AGPDGAGQVA KVCNNQLLAV |
| | UniProt | LMIGTAEAMA LGVANGLEAK |
| | Q915I6 | VLAEIMRRSS GGNWALEVYN PWPGVMENAP |
| | | ASRDYSGGFM AQLMAKDLGL AQEAAQASAS |
| | | STPMGSLALS LYRLLLKQGY AERDFSVVQK |
| | | LFDPTQGQ |
| 88 | Amyelois | MAGRATQCLLSIPKRGYSSKADKNVAFLGLGNMGGFM |
| | transitella | AANLVKKGFAVKGYDPSKEAVTAAAKNGITGAT |
| | NAD- | SIAAALEGADAVVSILPSNKVVLDAYLGKDGVVAHAPK |
| | dependent L- | GTLLIDSSTVDPNVPKQIFPVAIEKGVGFIDA |
| | serine | PVSGGTMGAQNATLAFMSGGRKEDFDRSLPMLKAMGA |
| | dehydrogenase | KQFHCGEIGAGQVAKLANNMLMGITGMATAECM |
| | NCBI | NMGIKMGLDPKVLLDVLNNSSARSWSTEVYCPVPGLVP |
| | XP_013199964.1 | TAPSSKNYDGGFKNELMVKDLELASGMALGIR |
| | | SPIPLGAVATQLYRMAQTRGFGQKDFSYIYQLLKEDKQ |
| 89 | Nicrophorus | MFQRTSVLLCRVERFAQTRTITNNVGFIGLGNMGSHMA |
| | vespilloides | NHLAKQGRKLKVFDVVADAAKSVPGAIVCKTP |
| | NAD- | QEAATDVSVVFTMLPDGNVVKDTVLRNEGIAKGIKKDA |
| | dependent L- | LMIDCSTIEPTTAKELHTIAKDNGYRFIDCPV |
| | serine | SGGVTGAAAGTLTYMIGGDIKDVDTARQYLLQAGKNIF |
| | dehydrogenase | HCGGPGAGQVAKLCNNLILGVTMAGTAESMNM |
| | NCBI | GLKYGLDPKVLTDIINVSTGRSWSSETYNPHPGILPNVPS |
| | XP_017776295.1 | SKNYDGGFMVKLIAKDLGLAEGAALAANAP |
| | | VPMTAAVHQLYRAMMNHGLGDKDFSVIYQFLQGKKF |
| 18 | Homo sapiens | MAHAMENSWT ISKEYHIDEE VGFALPNPQE |
| | Indoleamine | NLPDFYNDWM FIAKHLPDLI ESGQLRERVE |
| | 2,3- | KLNMLSIDHL TDHKSQRLAR LVLGCITMAY |
| | dioxygenase 1 | VWGKGHGDVR KVLPRNIAVP YCQLSKKLEL |
| | UniProt | PPILVYADCV LANWKKKDPN KPLTYENMDV |
| | P14902 | LFSFRDGDCS KGFFLVSLLV EIAAASAIKV |
| | | IPTVFKAMQM QERDTLLKAL |
| | | LEIASCLEKA LQVFHQIHDH VNPKAFFSVL |
| | | RIYLSGWKGN PQLSDGLVYE GFWEDPKEFA |
| | | GGSAGQSSVF QCFDVLLGIQ QTAGGGHAAQ |
| | | FLQDMRRYMP PAHRNFLCSL ESNPSVREFV |
| | | LSKGDAGLRE AYDACVKALV SLRSYHLQIV |
| | | TKYILIPASQ QPKENKTSED PSKLEAKGTG |
| | | GTDLMNFLKT VRSTTEKSLL |
| | | KEG |
| 90 | Mus musculus | MAYVWNRGDDDVRKVLPRNIAVPYCELSEKLGLPPILS |
| | Indoleamine | YADCVLANWKKKDPNGPMTYENMDILFSFPGG |
| | 2,3- | DCDKGFFLVSLLVEIAASPAIKAIPTVSSAVERQDLKALE |
| | dioxygenase 1 | KALHDIATSLEKAKEIFKRMRDFVDPDTFF |
| | NCBI | HVLRIYLSGWKCSSKLPEGLLYEGVWDTPKMFSGGSAG |
| | NP_001280619.1 | QSSIFQSLDVLLGIKHEAGKESPAEFLQEMRE |
| | | YMPPAHRNFLFFLESAPPVREFVISRHNEDLTKAYNECV |
| | | NGLVSVRKFHLAIVDTYIMKPSKKKPTDGDK |
| | | SEEPSNVESRGTGGTNPMTFLRSVKDTTEKALLSWP |
| 91 | Rattus | MPHSQISPAEGSRRILEEYHIDEDVGFALPHPLEELPDTY |
| | norvegicus | RPWILVARNLPKLIENGKLREEVEKLPTLR |
| | Indoleamine | TEELRGHRLQRLAHLALGYITMAYVWNRGDDDIRKVLP |
| | 2,3- | RNLAVPYCELSEKLGLPPILSYADCVLANWKK |
| | dioxygenase 1 | KDPNGPMTYENMDILFSFPGGDCDKGFFLVSLMVEIAAS |
| | NCBI | PAIKAIPTVSSAVEHQDPKALEKALCSIAAS |
| | NP_076463.1 | LEKAKEIFKRMRDFVDPDTFFHVLRIYLSGWKGNPKLPE |
| | | GLLYEGVWDTPKKFSGGSAGQSSIFQSLDVL |
| | | LGIKHDVGEGSAAEFLQEMREYMPPAHRNFLSSLESAPP |
| | | VREFVILRRNEDLKEAYNECVNGLVSLRMFH |
| | | LSIVDTYIVKPSKQKPMGGHKSEEPSNTENRGTGGTDV |
| | | MNFLRSVKDTTKKALLSWP |
| 19 | Anabaena | MKTLSQAQSKTSSQQFSFTGNSSANVIIGNQKLTINDVA |
| | variabilis | RVARNGTLVSLTNNTDILQGIQASCDYINNAVESGEPIY |
| | Phenylalanine | GVTSGFGGMANVAISREQASELQTNLVWFLKTGAGNK |
| | ammonia | LPLADVRAAMLLRANSHMRGASGIRLELIKRMEIFLNA |
| | lyase | GVTPYVYEFGSIGASGDLVPLSYITGSLIGLDPSFKVDFN |
| | UniProt | GKEMDAPTALRQLNLSPLTLLPKEGLAMMNGTSVMTGI |
| | Q3M5Z3 | AANCVYDTQILTAIAMGVHALDIQALNGTNQSFHPFIHN |
| | | SKPHPGQLWAADQMISLLANSQLVRDELDGKHDYRDH |
| | | ELIQDRYSLRCLPQYLGPIVDGISQIAKQIEIEINSVTDNPL |
| | | IDVDNQASYHGGNFLGQYVGMGMDHLRYYIGLLAKHL |
| | | DVQIALLASPEFSNGLPPSLLGNRERKVNMGLKGLQICG |
| | | NSIMPLLTFYGNSIADRFPTHAEQFNQNINSQGYTSATLA |
| | | RRSVDIFQNYVAIALMFGVQAVDLRTYKKTGHYDARA |
| | | CLSPATERLYSAVRHVVGQKPTSDRPYIWNDNEQGLDE |
| | | HIARISADIAAGGVIVQAVQDILPCLH |
| 92 | Arabidopsis | MDQIEAMLCGGGEKTKVAVTTKTLADPLNWGLAADQ |
| | thaliana | MKGSHLDEVKKMVEEYRRPVVNLGGETLTIGQVA |
| | Phenylalanine | AISTVGGSVKVELAETSRAGVKASSDWVMESMNKGTD |
| | ammonia | SYGVTTGFGATSHRRTKNGTALQTELIRFLNAG |
| | lyase 2 | IFGNTKETCHTLPQSATRAAMLVRVNTLLQGYSGIRFEIL |
| | NCBI | EAITSLLNHNISPSLPLRGTITASGDLVPL |
| | NP_190894.1 | SYIAGLLTGRPNSKATGPDGESLTAKEAFEKAGISTGFFD |
| | | LQPKEGLALVNGTAVGSGMASMVLFEANVQ |
| | | AVLAEVLSAIFAEVMSGKPEFTDHLTHRLKHHPGQIEAA |
| | | AIMEHILDGSSYMKLAQKVHEMDPLQKPKQD |
| | | RYALRTSPQWLGPQIEVIRQATKSIEREINSVNDNPLIDV |
| | | SRNKAIHGGNFQGTPIGVSMDNTRLAIAAI |
| | | GKLMFAQFSELVNDFYNNGLPSNLTASSNPSLDYGFKG |
| | | AEIAMASYCSELQYLANPVTSHVQSAEQHNQD |
| | | VNSLGLISSRKTSEAVDILKLMSTTFLVGICQAVDLRHLE |
| | | ENLRQTVKNTVSQVAKKVLTTGINGELHPS |
| | | RFCEKDLLKVVDREQVFTYVDDPCSATYPLMQRLRQVI |
| | | VDHALSNGETEKNAVTSIFQKIGAFEEELKAV |
| | | LPKEVEAARAAYGNGTAPIPNRIKECRSYPLYRFVREEL |
| | | GTKLLTGEKVVSPGEEFDKVFTAMCEGKLID |
| | | PLMDCLKEWNGAPIPIC |
| 93 | Pseudomonas | MRPIERLLAVVDGEVSARLDEGMRGRIDAGHALLLELIA |
| | putida | AGAPIYGVTTGLGAAVDHAQGDAGFQQRIAA |
| | Phenylalanine | GRAVGVGRLASRREVRAIMAARLAGLALGRSGISLASA |
| | ammonia | MALGDFLDHGIHPEVPLLGSLGASDLAPLAHV |
| | lyase | TLALQGQGWVEYHGERLPAAEALQRAGLAPLVPRDKD |
| | NCBI | GLALVSANSASIGLGALLVSETQRLLDRQRGVL |
| | WP_064302405.1 | ALSCEGYRAGVAPFQAAHLRPAPGLVEESTALLALLEG |
| | | GDRQARRLQDPLSFRCSTVVLGAVRDALARAR |
| | | DIVVIELQSGADNPALVVKSREVLVTANFDSTHLALAFE |
| | | GLGLALSRLAVASAERMAKLLSPGSSELPHS |
| | | LSPRPGSVGLAALQRTAAALVAEIVHLANPLPALSVPVA |
| | | DRVEDYAGQGLAVVEKTARLVQRVEWLVRIE |
| | | AVVAAQAVDLRAGITLGSEASAIYRQIRQVVAFVEDDR |
| | | AIDVTGEFWGR |
| 116 | Homo sapiens | MAKQLQARRLDGIDYNPWVEFVKLASEHDVVNLGQGF |
| | KYAT1 | PDFPPPDFAVEAFQHAVSGDFMLNQYTKTFGYP |
| | Glutamine- | PLTKILASFFGELLGQEIDPLRNVLVTVGGYGALFTAFQ |
| | pyruvate | ALVDEGDEVIIIEPFFDCYEPMTMMAGGRPV |
| | transaminase | FVSLKPGPIQNGELGSSSNWQLDPMELAGKFTSRTKALV |
| | NCBI | LNTPNNPLGKVFSREELELVASLCQQHDVVC |
| | NP_001116143.1 | ITDEVYQWMVYDGHQHISIASLPGMWERTLTIGSAGKT |
| | | FSATGWKVGWVLGPDHIMKHLRTVHQNSVFHC |
| | | PTQSQAAVAESFEREQLLFRQPSSYFVQFPQAMQRCRD |
| | | HMIRSLQSVGLKPIIPQGSYFLITDISDFKRK |
| | | MPDLPGAVDEPYDRRFVKWMIKNKGLVAIPVSIFYSVP |
| | | HQKHFDHYIRFCFVKDEATLQAMDEKLRKWKV |
| | | EL |
| 117 | Mus musculus | MSKQLQARRLEGIDHNPWVEFTRLSKEYDVVNLGQGFP |
| | KYAT1 | DFSPPDFAVQAFQQATTGNFMLNQYTSAFGYP |
| | Glutamine- | PLTKILASFFGKLLGQEMDPLKNVLVTVGAYGALFTAF |
| | pyruvate | QALVDEGDEVIIIEPAFNCYEPMTMMAGGRPV |
| | transaminase | FVSLRLSPAPKGQLGSSNDWQLDPTELASKFTPRTKILV |
| | NCBI | LNTPNNPLGKVFSKKELELVAALCQQHDVLC |
| | NP_001343403.1 | FSDEVYQWLVYDGHQHISIASLPGMWERTLTIGSAGKSF |
| | | SATGWKVGWVMGPDNIMKHLRTVHQNSIFHC |
| | | PTQAQAAVAQCFEREQQHFGQPSSYFLQLPQAMGLNRD |
| | | HMIQSLQSVGLKPLIPQGSYFLIADISDFKSS |
| | | MPDLPGAMDEPYDTRFAKWMIKNKGLSAIPVSTFYSQP |
| | | HHKDFDHYIRFCFVKDKATLQAMDKRLCSWKG |
| | | EPQA |
| 118 | Homo sapiens | MKDCSNGCSAECTGEGGSKEVVGTFKAKDLIVTPATIL |
| | Branched- | KEKPDPNNLVFGTVFTDHMLTVEWSSEFGWEK |
| | chain-amino- | PHIKPLQNLSLHPGSSALHYAVEVFDKEELLECIQQLVK |
| | acid | LDQEWVPYSTSASLYIRPTFIGTEPSLGVKK |
| | transaminase 1 | PTKALLFVLLSPVGPYFSSGTFNPVSLWANPKYVRAWK |
| | NCBI | GGTGDCKMGGNYGSSLFAQCEAVDNGCQQVLW |
| | NP_001171562.1 | LYGEDHQITEVGTMNLFLYWINEDGEEELATPPLDGIILP |
| | | GVTRRCILDLAHQWGEFKVSERYLTMDDLT |
| | | TALEGNRVREMFGSGTACVVCPVSDILYKGETIHIPTME |
| | | NGPKLASRILSKLTDIQYGREESDWTIVLS |
| 119 | Saccharomyces | MTLAPLDASKVKITTTQHASKPKPNSELVFGKSFTDHML |
| | cerevisiae | TAEWTAEKGWGTPEIKPYQNLSLDPSAVVFH |
| | Branched- | YAFELFEGMKAYRTVDNKITMFRPDMNMKRMNKSAQ |
| | chain-amino- | RICLPTFDPEELITLIGKLIQQDKCLVPEGKGYS |
| | acid | LYIRPTLIGTTAGLGVSTPDRALLYVICCPVGPYYKTGFK |
| | transaminase | AVRLEATDYATRAWPGGCGDKKLGANYAPC |
| | NCBI | VLPQLQAASRGYQQNLWLFGPNNNITEVGTMNAFFVFK |
| | NP_012682.1 | DSKTGKKELVTAPLDGTILEGVTRDSILNLAK |
| | | ERLEPSEWTISERYFTIGEVTERSKNGELLEAFGSGTAAI |
| | | VSPIKEIGWKGEQINIPLLPGEQTGPLAKE |
| | | VAQWINGIQYGETEHGNWSRVVTDLN |
| 120 | Escherichia | MTVTRPRAERGAFPPGTEHYGRSLLGAPLIWFPAPAASR |
| | coli murein | ESGLILAGTHGDENSSVVTLSCALRTLTPSL |
| | peptide | RRHHVVLCVNPDGCQLGLRANANGVDLNRNFPAANW |
| | amidase A | KEGETVYRWNSAAEERDVVLLTGDKPGSEPETQA |
| | (Amidase) | LCQLIHRIQPAWVVSFHDPLACIEDPRHSELGEWLAQAF |
| | NCBI | ELPLVTSVGYETPGSFGSWCADLNLHCITAE |
| | NP_309932.2 | FPPISSDEASEKYLFAMANLLRWHPKDAIRPS |
| 121 | Pseudomonas | MGWGLRLRTLLTGVMILLACQVGEVLAAAQIKSVRIWR |
| | aeruginosa | APDNTRLVFDLSGPVQHSLFTLAAPNRIVIDV |
| | PA01 | SGAQLATQLNGLKLGNTPITAVRSAQRTPNDLRMVLDL |
| | (Amidase) | SAQVTPKSFVLPPNQQYGNRLVVDLYDQGADL |
| | NCBI | TPDVPATPTPSVPVTPVTPTQPVAKLPLPTKGGTRDIVIAI |
| | NP_253634.1 | DAGHGGEDPGALGPGGLHEKNITLSIARE |
| | | LQRQINQVRGYRAELTRTGDYFIPLRKRTEIARKKGADL |
| | | FVSIHADAAPSRSAFGASVFALSDRGATSET |
| | | ARWLADSENRSDLIGGDGSVSLGDKDQMLAGVLLDLS |
| | | MTATLSSSLDVGHKVLTNVGRITSLHKRRVEQA |
| | | GFMVLKSPDIPSILVETGFISNVNESRKLASASHQQALAR |
| | | SITSGIRQYFQQSPPPGTYIASLRAQGKLS |
| | | MGPREHVVRPGETLAMIAQRYEVSMAALRSSNSLSSDN |
| | | LKVGQALSIPSTALAAQ |
| 122 | Escherichia | MKVLIVESEFLHQDTWVGNAVERLADALSQQNVTVIKS |
| | coli | TSFDDGFAILSSNEAIDCLMFSYQMEHPDEHQ |
| | Arginine | NVRQLIGKLHERQQNVPVFLLGDREKALAAMDRDLLEL |
| | decarboxylase | VDEFAWILEDTADFIAGRAVAAMTRYRQQLLP |
| | NCBI | PLFSALMKYSDIHEYSWAAPGHQGGVGFTKTPAGRFYH |
| | NP_418541.2 | DYYGENLFRTDMGIERTSLGSLLDHTGAFGES |
| | | EKYAARVFGADRSWSVVVGTSGSNRTIMQACMTDNDV |
| | | VVVDRNCHKSIEQGLMLTGAKPVYMVPSRNRYG |
| | | IIGPIYPQEMQPETLQKKISESPLTKDKAGQKPSYCVVTN |
| | | CTYDGVCYNAKEAQDLLEKTSDRLHFDEAW |
| | | YGYARFNPIYADHYAMRGEPGDHNGPTVFATHSTHKLL |
| | | NALSQASYIHVREGRGAINFSRFNQAYMMHAT |
| | | TSPLYAICASNDVAVSMMDGNSGLSLTQEVIDEAVDFR |
| | | QAMARLYKEFTADGSWFFKPWNKEVVTDPQTG |
| | | KTYDFADAPTKLLTTVQDCWVMHPGESWHGFKDIPDN |
| | | WSMLDPIKVSILAPGMGEDGELEETGVPAALVT |
| | | AWLGRHGIVPTRTTDFQIMFLFSMGVTRGKWGTLVNTL |
| | | CSFKRHYDANTPLAQVMPELVEQYPDTYANMG |
| | | IHDLGDTMFAWLKENNPGARLNEAYSGLPVAEVTPREA |
| | | YNAIVDNNVELVSIENLPGRIAANSVIPYPPG |
| | | IPMLLSGENFGDKNSPQVSYLRSLQSWDHHFPGFEHETE |
| | | GTEIIDGIYHVMCVKA |
| 123 | Pseudomonas | MAARRTRKDDGSNWTVADSRSIYGIRHWGAGYYAIND |
| | Arginine | AGNVEVRPQGADAQPIDLHGLVEQLREAGLSLP |
| | decarboxylase | LLVRFPDILQDRVRKLTGAFDANIARLEYGSRYTALYPI |
| | NCBI | KVNQQEAVVESIIATQNVSIGLEAGSKPELM |
| | WP_015479045.1 | AVLALAPKGGTIVCNGYKDREFIKLALMGQKLGHNVFI |
| | | VIEKESEVQLVIEEAANVGVLPQVGLRVRLSS |
| | | LASSKWADTGGEKAKFGLSAAQLLSVVERFRAAGLDQ |
| | | GVRLLHFHMGSQIANLADYQHGFKEAIRYYGEL |
| | | RALGLPVDHVDVGGGLGVDYDGTHSRNASSINYDIDDY |
| | | AGVVVGMLKEFCDAQGLPHPHIFSESGRALTA |
| | | HHAVLITQVTDVERHNDEVPKITDLAEQPEIVQWLADLL |
| | | GPTDAEMVTETYWRATHYMSDAAAQYAEGKI |
| | | SLAQKALAEQCYFAICRRLHNQLKAHQRSHRQVLDELN |
| | | DKLADKYICNFSVFQSLPDTWAIGQVLPILPI |
| | | HRLGEEPTRRAVLQDLTCDSDGKITQYVDEQSIETSLPV |
| | | HEVKEGEEYMIGVFLVGAYQEILGDMHNLFG |
| | | DTDSVNVYQNADGSFYHAGIETHDTIEDMLRYVHLSPE |
| | | ELMTHYRDKVAGAKLTARERTQFLDALRLGLT |
| | | RSAYLSS |
| 124 | Homo sapiens | MNASEFRRRGKEMVDYVANYMEGIEGRQVYPDVEPGY |
| | Aromatic-L- | LRPLIPAAAPQEPDTFEDIINDVEKIIMPGVTH |
| | amino-acid | WHSPYFFAYFPTASSYPAMLADMLCGAIGCIGFSWAAS |
| | decarboxylase | PACTELETVMMDWLGKMLELPKAFLNEKAGEG |
| | NCBI | GGVIQGSASEATLVALLAARTKVIHRLQAASPELTQAAI |
| | NP_000781.1 | MEKLVAYSSDQAHSSVERAGLIGGVKLKAIP |
| | | SDGNFAMRASALQEALERDKAAGLIPFFMVATLGTTTC |
| | | CSFDNLLEVGPICNKEDIWLHVDAAYAGSAFI |
| | | CPEFRHLLNGVEFADSFNFNPHKWLLVNFDCSAMWVK |
| | | KRTDLTGAFRLDPTYLKHSHQDSGLITDYRHWQ |
| | | IPLGRRFRSLKMWFVFRMYGVKGLQAYIRKHVQLSHEF |
| | | ESLVRQDPRFEICVEVILGLVCFRLKGSNKVN |
| | | EALLQRINSAKKIHLVPCHLRDKFVLRFAICSRTVESAHV |
| | | QRAWEHIKELAADVLRAERE |
| 125 | Drosophila | MEAPEFKDFAKTMVDFIAEYLENIRERRVLPEVKPGYLK |
| | melanogaster | PLIPDAAPEKPEKWQDVMQDIERVIMPGVTH |
| | Aromatic-L- | WHSPKFHAYFPTANSYPAIVADMLSGAIACIGFTWIASP |
| | amino-acid | ACTELEVVMMDWLGKMLELPAEFLACSGGKG |
| | decarboxylase | GGVIQGTASESTLVALLGAKAKKLKEVKELHPEWDEHT |
| | NCBI | ILGKLVGYCSDQAHSSVERAGLLGGVKLRSVQ |
| | NP_724164.1 | SENHRMRGAALEKAIEQDVAEGLIPFYAVVTLGTTNSC |
| | | AFDYLDECGPVGNKHNLWIHVDAAYAGSAFIC |
| | | PEYRHLMKGIESADSFNFNPHKWMLVNFDCSAMWLKD |
| | | PSWVVNAFNVDPLYLKHDMQGSAPDYRHWQIPL |
| | | GRRFRALKLWFVLRLYGVENLQAHIRRHCNFAKQFGDL |
| | | CVADSRFELAAEINMGLVCFRLKGSNERNEAL |
| | | LKRINGRGHIHLVPAKIKDVYFLRMAICSRFTQSEDMEY |
| | | SWKEVSAAADEMEQEQ |
| 126 | Microcystis | MQNLDENSLDYAVSKPDLAEQRQEFRGLSNKVYFNFG |
| | aeruginosa | GQGTLPKAGLEAIIDAHNFLQQKGPFSGRVNDW |
| | Cysteine lyase | ITGKTELLRQEMAQELGISPSTLSITEDVTVGCNIALWGV |
| | NCBI | DWQAGEHILLTDCEHPGIIATVQEIARRYH |
| | WP_012267325.1 | LEISTCPIRETLNGGNPIEVISAHLRPKTRVLVVSHVLWN |
| | | TGQVLPLKEISQLCHDNSVTEKPVLVVVDA |
| | | AQSVGCLPLDLSATAADCYAFTGHKWWCGPAGVGGLY |
| | | IRPEIFPSLQPTFIGWRGIETDNRGQPIGWKPD |
| | | ARRFEVATSAYPQFEGLRATIAVHNAWGDGGQRYEKIC |
| | | QLAAYLWEELKTIKGVKCLKNSPPESGLVSFQ |
| | | IDSAITPQNLVQQLEKQGFLLRTLLDPLCVRACVHYFTL |
| | | PSEIEQLVAAVKKLV |
| 127 | Planktothrix | MTISSLKTHRQQFPALTNKAYFNYGGQGPLAQISMDAIF |
| | agardhii | EGYKYMQSHGPFSGKVNQWQNQETQLTRHLL |
| | Cysteine lyase | ATELGISPETLTFTENVTVGCNIALWGIDWQPGDHLLISD |
| | NCBI | CEHPGILAIIQEIQRRFYLEVSFFPLRETL |
| | WP_042153795.1 | NQNDPVGMISEYLKPHTRLLVISHILWNTGQVLPLTEIV |
| | | NLCHNNYNTKVLVDAAQSVGVLPINLTETGV |
| | | DFYAFTGHKWFCGPDGLGGLYVSTQSRSELSPTFIGWRS |
| | | IIGDEQGKPISWTPDGKRYEVATSAYPLYAA |
| | | LRHAIALHHQWGTAIERYQQICQNSQYLWQKLSEIPQIQ |
| | | CLKTSPPEAGLVSFQLTNGKSHKSLVNTLEN |
| | | QGIFLRTLLDPNCVRACVHYFTLSSEIDQLIDAINQFIAVS |
| 128 | Homo sapiens | MASESGKLWGGRFVGAVDPIMEKFNASIAYDRHLWEV |
| | Arginino succinate | DVQGSKAYSRGLEKAGLLTKAEMDQILHGLDKV |
| | lyase | AEEWAQGTFKLNSNDEDIHTANERRLKELIGATAGKLH |
| | NCBI | TGRSRNDQVVTDLRLWMRQTCSTLSGLLWELI |
| | NP_000039.2 | RTMVDRAEAERDVLFPGYTHLQRAQPIRWSHWILSHAV |
| | | ALTRDSERLLEVRKRINVLPLGSGAIAGNPLG |
| | | VDRELLRAELNFGAITLNSMDATSERDFVAEFLFWASLC |
| | | MTHLSRMAEDLILYCTKEFSFVQLSDAYSTG |
| | | SSLMPQKKNPDSLELIRSKAGRVFGRCAGLLMTLKGLPS |
| | | TYNKDLQEDKEAVFEVSDTMSAVLQVATGVI |
| | | STLQIHQENMGQALSPDMLATDLAYYLVRKGMPFRQA |
| | | HEASGKAVFMAETKGVALNQLSLQELQTISPLF |
| | | SGDVICVWDYGHSVEQYGALGGTARSSVDWQIRQVRA |
| | | LLQAQQA |
| 129 | Mus musculus | MASESGKLWGGRFVGAVDPIMEKFNSSISYDRHLWNV |
| | Argininosuccinate | DVQGSKAYSRGLEKAGLLTKAEMQQILQGLDKV |
| | lyase | AEEWAQGTFKLHPNDEDIHTANERRLKELIGEAAGKLH |
| | NCBI | TGRSRNDQVVTDLRLWMRQTCSKLSALLRVLI |
| | NP_598529.1 | GTMVDRAEAERDVLFPGYTHLQRAQPIRWSHWILSHAV |
| | | ALTRDSERLLEVQKRINVLPLGSGAIAGNPLG |
| | | VDRELLRAELNFGAITLNSMDATSERDFVAEFLFWASLC |
| | | MTHLSRMAEDLILYGTKEFSFVQLSDAYSTG |
| | | SSLMPQKKNPDSLELIRSKAGRVFGRCAGLLMTLKGLPS |
| | | TYNKDLQEDKEAVFEVSDTMIAVLQVATGVI |
| | | STLQIHRENMKQALSPDMLATDLAYYLVRKGMPFRQA |
| | | HEASGKAVFMAETKGVALNLLSLQELQTISPLF |
| | | SGDVSHVWDYSHSVEQYSALGGTAKSSVEWQIRQVRA |
| | | LLQAQEP |

TABLE 4
Exemplary amino acid sequences of transmembrane
domains, tags, and signal peptides.

| SEQ | | | |
|---|---|---|---|
| ID | Sequence | Sequence | |
| NO: | name | description | Amino acid sequence |
| 20 | GPA | Fragment of GPA | LSTTEVAMHTSTSSSVTKSYISSQTNDTHKR |
| | | comprising a | DTYAATPRAHEVSEISVRTVYPPEEETGERV |
| | | transmembrane | QLAHHFSEPEITLIIFGVMAGVIGTILLISYGIR |
| | | domain | RLIKKSPSDVKPLPSPDTDVPLSSVEIENPETS |
| | | | DQ |
| 21 | HA | HA epitope tag | YPYDVPDYA |
| 22 | Signal | Signal peptide | MYGKIIFVLLLSEIVSISA |
| | peptide | | |
| 24 | CD71 | Fragment of CD71 | ICYGTIAVIVFFLIGFMIGYLGYC |
| | (66-89) | comprising a | |
| | | transmembrane | |
| | | domain | |
| 26 | Kell | Amino acids 1-79 of | MEGGDQSEEEPRERSQAGGMGTLWSQESTP |
| | | Kell, comprising a | EERLPVEGSRPWAVARRVLTAILILGLLLCFS |
| | | transmembrane | VLLFYNFQNCGPRPCET |
| | | domain | |
| 27 | SMIM1 | SMIM1 | MQPQESHVHYSRWEDGSRDGVSLGAVSSTE |
| | | | EASRCRRISQRLCTGKLGIAMKVLGGVALF |
| | | | WIIFILGYLTGYYVHKCK |

TABLE 5
Exemplary amino acid sequences of linker regions.

| SEQ ID | Anchor | | |
|---|---|---|---|
| NO: | name | Anchor description | Amino acid sequence |
| 28 | Linker | Linker region | [symbol] YPYDVPDYA [symbol] |
| | | including | |
| | | hemagglutinin A | |
| | | (HA) epitope tag | |
| | | (linker shown in | |
| | | italics and | |
| | | underlined) | |
| 29 | Linker | Linker region | GGGGSGRGGSGRGGSGRGGSGRGGRR |
| 30 | Linker | Linker region | [symbol] YPYDVPDYA [symbol] |
| | | including HA | |
| | | epitope tag (linker | |
| | | shown in italics and | |
| | | underlined) | |
| 31 | Linker | Linker region | [symbol] YPYDVPDYA [symbol] |
| | | including HA | |
| | | epitope tag (linker | |
| | | shown in italics and | |
| | | underlined) | |

TABLE 6
Exemplary amino acid sequences of cell
surface markers bound by cell targeting moieties

| SEQ | | |
|---|---|---|
| ID | Sequence name | |
| NO: | and source | Amino acid sequence |
| 32 | Myeloid cell | MPLLLLLPLL WAGALAMDPN FWLQVQESVT |
| | surface antigen | VQEGLCVLVP CTFFHPIPYYDKNSPVHGYW FREGAIISRD |
| | CD33 | SPVATNKLDQ EVQEETQGRF RLLGDPSRNNCSLSIVDARR |
| | UniProt P20138 | RDNGSYFFRM ERGSTKYSYK SPQLSVHVTD LTHRPKILIP |
| | | GTLEPGHSKN LTCSVSWACE QGTPPIFSWL SAAPTSLGPR |
| | | TTHSSVLIIT PRPQDHGTNL TCQVKFAGAG VTTERTIQLN |
| | | VTYVPQNPTT GIFPGDGSGK QETRAGVVHG AIGGAGVTAL |
| | | LALCLCLIFF IVKTHRRKAA RTAVGRNDTH PTTGSASPKH |
| | | QKKSKLHGPT ETSSCSGAAP TVEMDEELHY |
| | | ASLNFHGMNP |
| | | SKDTSTEYSE VRTQ |
| 33 | B-lymphocyte | MTTPRNSVNG TFPAEPMKGP IAMQSGPKPL FRRMSSLVGP |
| | antigen CD20 | TQSFFMRESK TLGAVQIMNG LFHIALGGLL MIPAGIYAPI |
| | UniProt P11836 | CVTVWYPLWG GIMYIISGSL LAATEKNSRK |
| | | CLVKGKMIMN SLSLFAAISG MILSIMDILN IKISHFLKME |
| | | SLNFIRAHTP YINIYNCEPA NPSEKNSPST QYCYSIQSLF |
| | | LGILSVMLIF |
| | | AFFQELVIAG IVENEWKRTC SRPKSNIVLL SAEEKKEQTI |
| | | EIKEEVVGLT ETSSQPKNEE DIEIIPIQEE EEEETETNFP |
| | | EPPQDQESSP IENDSSP |
| 34 | T-cell surface | MNRGVPFRHL LLVLQLALLP AATQGKKVVL |
| | glycoprotein | GKKGDTVELT CTASQKKSIQ FHWKNSNQIK ILGNQGSFLT |
| | CD4 | KGPSKLNDRA DSRRSLWDQG NFPLIIKNLK IEDSDTYICE |
| | UniProt P01730 | VEDQKEEVQL LVFGLTANSD THLLQGQSLT LTLESPPGSS |
| | | PSVQCRSPRG KNIQGGKTLS VSQLELQDSG TWTCTVLQNQ |
| | | KKVEFKIDIV VLAFQKASSI VYKKEGEQVE FSFPLAFTVE |
| | | KLTGSGELWW QAERASSSKS WITFDLKNKE |
| | | VSVKRVTQDP KLQMGKKLPL HLTLPQALPQ |
| | | YAGSGNLTLA LEAKTGKLHQ EVNLVVMRAT |
| | | QLQKNLTCEV WGPTSPKLML LKLENKEAK |
| | | VSKREKAVWV LNPEAGMWQC LLSDSGQVLL |
| | | SNIKVLPTW STPVQPMALI VLGGVAGLLL FIGLGIFFCV |
| | | RCRHRRRQAE RMSQIKRLLS EKKTCQCPHR FQKTCSPI |
| 35 | B cell | QNEYFDSLLH ACXFCQLRCS SNTPPLTCQR YCNASVTNSV |
| | maturation | KGTNAILWTC LGLSLIISLA VFVLMFLLRK ISSEPLKDEF |
| | antigen | KNTEMESHSV AQAGVQXRXL NSLQPXPXGX |
| | (BCMA) | KQXSHLSLLS XPDYRIRSPG HG |
| | UniProt | |
| | A7KBT6 | |
| 36 | Prostate- | MWVPVVFLTL SVTWIGAAPL ILSRIVGGWE |
| | specific antigen | CEKHSQPWQV LVASRGRAVC GGVLVHPQWV |
| | (PSA) | LTAAHCIRNK SVILLGRHSL FHPEDTGQVF QVSHSFPHPL |
| | UniProt | YDMSLLKNRF LRPGDDSSIE PEEFLTPKKL QCVDLHVISN |
| | Q8NCW4 | DVCAQVHPQKVTKFMLCAGR WTGGKSTCSG |
| | | DSGGPLVCNG VLQGITSWGS EPCALPERPS |
| | | LYTKVVHYRK WIKDTIVANP |
| 37 | Tumor necrosis | MLQMAGQCSQ NEYFDSLLHA CIPCQLRCSS NTPPLTCQRY |
| | factor receptor | CNASVTNSVK GTNAILWTCL GLSLIISLAV FVLMFLLRKI |
| | superfamily | NSEPLKDEFK NTGSGLLGMA NIDLEKSRTG DEIILPRGLE |
| | member 17 | YTVEECTCED CIKSKPKVDS DHCFPLPAME EGATILVTTK |
| | (CD269) | TNDYCKSLPA ALSATEIEKS ISAR |
| | UniProt | |
| | Q02223 | |
| 38 | Interleukin-3 | MVLLWLTLLL IALPCLLQTK EDPNPPITNL RMKAKAQQLT |
| | receptor subunit | WDLNRNVTDI ECVKDADYSM PAVNNSYCQF |
| | alpha (CD123) | GAISLCEVTN YTVRVANPPF STWILFPENS GKPWAGAENL |
| | UniProt P26951 | TCWIHDVDFL SCSWAVGPGA PADVQYDLYL |
| | | NVANRRQQYE CLHYKTDAQG TRIGCRFDDI SRLSSGSQSS |
| | | HILVRGRSAA FGIPCTDKFV VFSQIEILTP PNMTAKCNKT |
| | | HSFMHWKMRS HFNRKFRYEL QIQKRMQPVI |
| | | TEQVRDRTSF QLLNPGTYTV QIRARERVYE FLSAWSTPQR |
| | | FECDQEEGAN TRAWRTSLLI ALGTLLALVC VFVICRRYLV |
| | | MQRLFPRIPH MKDPIGDSFQ |
| | | NDKLVVWEAG KAGLEECLVT EVQVVQKT |
| 39 | T-cell-specific | MLRLLLALNL FPSIQVTGNK ILVKQSPMLV AYDNAVNLSC |
| | surface | KYSYNLFSRE FRASLHKGLD SAVEVCVVYG |
| | glycoprotein | NYSQQLQVYS KTGFNCDGKL GNESVTFYLQ |
| | CD28 | NLYVNQTDIY FCKIEVMYPP PYLDNEKSNG TIIHVKGKHL |
| | UniProt P10747 | CPSPLFPGPS KPFWVLVVVG GVLACYSLLV TVAFIIFWVR |
| | | SKRSRLLHSD YMNMTPRRPG |
| | | PTRKHYQPYA PPRDFAAYRS |
| 94 | CD47 | MWPLVAALLL GSACCGSAQL LFNKTKSVEF TFCNDTVVIP |
| | UniProt | CFVTNMEAQN TTEVYVKWKF KGRDIYTFDG |
| | Q08722 | ALNKSTVPTD FSSAKIEVSQ LLKGDASLKM |
| | | DKSDAVSHTG NYTCEVTELT REGETIIELK YRVVSWFSPN |
| | | ENILIVIFPI FAILLFWGQF GIKTLKYRSG GMDEKTIALL |
| | | VAGLVITVIV IVGAILFVPG EYSLKNATGL GLIVTSTGIL |
| | | ILLHYYVFST AIGLTSFVIA ILVIQVIAYI LAVVGLSLCI |
| | | AACIPMHGPL LISGLSILAL AQLLGLVYMK FVASNQKTIQ |
| | | PPRKAVEEPL NAFKESKGMM NDE |

TABLE 7
Exemplary amino acid sequences for targeting single-chain variable fragments
(scFvs) for cell targeting moieties

| SEQ | Sequence | | |
|---|---|---|---|
| ID | name | Source | Amino acid sequence |
| 95 | Humanized | USPN | QVQLVQSGAEVKKPGASVKVSCKASGYTFTNYD |
| | Anti- | 9587019 | INWVRQAPGQGLEWIGWIYPGDGSTKY |
| | human | | NEKFKAKATLTADTSTSTAYMELRSLRSDDTAV |
| | CD33 #1 | | YYCASGYEDAMDYWGQGTTVTVSS |
| | VH | | |
| 96 | Humanized | USPN | DIQMTQSPSSLSASVGDRVTINCKASQDINSYLS |
| | Anti- | 9587019 | WFQQKPGKAPKTLIYRANRLVDGVPS |
| | human | | RFSGSGSGQDYTLTISSLQPEDFATYYCLQYDEFP |
| | CD33 #1 | | LTFGGGTKVEIK |
| | VL | | |
| 97 | Humanized | USPN | EVKLQESGPELVKPGASVKMSCKASGYKFTDYV |
| | Anti- | 8759494 | VHWLKQKPGQGLEWIGYINPYNDGTKY |
| | human | | NEKFKGKATLTSDKSSSTAYMEVSSLTSEDSAVY |
| | CD33 #2 | | YCARDYRYEVYGMDYWGQGTSVTVSS |
| | VH | | |
| 98 | Humanized | USPN | DIVLTQSPTIMSASPGERVTMTCTASSSVNYIHW |
| | Anti- | 8759494 | YQQKSGDSPKRWIFDTSKVASGVPAR |
| | human | | FSGSGSGTSYSLTISTMEAEDAATYYCQQWRSYP |
| | CD33 #2 | | LTFGDGTRLELKRADAAPTVS |
| | VL | | |
| 99 | Humanized | USPN | EVQLQQSGAELVRPGASVKLSCKASGYTFTNYW |
| | Anti- | 9951133 | MNWVKQRPGQGLEWIGMIDPSDNETHY |
| | human | | SQMFKDKATLTVDKSSSTAYMQLISLTSEDSAVY |
| | CD33 #3 | | YCAGYYGNFGWFVYWGQGTLVTVSA |
| | VH | | |
| 100 | Humanized | USPN | DIFMTQTPLSLPVSLGDPASISCRSSQTIVHSNGNT |
| | Anti- | 9951133 | YLEWYLQKPGQSPKLLIYKVSNRF |
| | human | | SGVPDRFSGSGSGTDFTLKISRVEAEDLGVYYCF |
| | CD33 #3 | | QGSHVPPTFGGGTKVEIK |
| | VL | | |
| 101 | Humanized | USPN | EVQLQQSGAELVKPGASVKMSCKAFGYTFTTFPI |
| | Anti- | 9951133 | EWMKQSHGKSLEWIGNFHPYNDQTKY |
| | human | | NEEFKGRAKLTIDRSSSTVYLELGRLTSDDSAVY |
| | CD33 #4 | | YCARGYYYAFDFWGQGTTLTVSS |
| | VH | | |
| 102 | Humanized | USPN | DIQMTQSPASLTVSLGQRATISCRASESVDSYGNS |
| | Anti- | 9951133 | YLHWYQQKPGQPPQLLIYLASNLES |
| | human | | GVPARFSGSGSRTDFTLTIDPVEADDAATYYCQQ |
| | CD33 #4 | | NNEDPWTFGGGTKVEIK |
| | VL | | |
| 40 | anti- | IMGT/ | EVQLVQSGAEVKKPGSSVKVSCKASGYTITDSNI |
| | Myeloid | 2Dstructure- | HWVRQAPGQSLEWIGYIYPYNGGTDYNQKFKN |
| | cell | DB card | RATLTVDNPT |
| | surface | for INN 10315 | NTAYMELSSLRSEDTAFYYCVNGNPWLAYWGQ |
| | antigen | VH region | GTLVTVSS [symbol] [symbol] DIQLTQSPSTL |
| | CD33 | linked to the | SASVGDRVTITCRASESLDNYGIRFLTWFQQKPG |
| | scFv | VL region | KAPKLLMYAASNQGSGVPSRFSGSGSGTEFTLTI |
| | | (linker region | SSLQPDDFATYYCQQTKEVPWSFGQGTKVEVK |
| | | show in | |
| | | italics and | |
| | | underlined) | |
| 103 | anti- | | EVQLVQSGAEVKKPGSSVKVSCKASGYTITDSNIHWVR |
| | Myeloid | | QAPGQSLEWIGYIYPYNGGTDYNQKFKNRATLTVDNPT |
| | cell | | NTAYMELSSLRSEDTAFYYCVNGNPWLAYWGQGTLVTV |
| | surface | | SS |
| | antigen | | GGGGSGGGGSGGGGS |
| | CD33 | | DIQLTQSPSTLSASVGDRVTITCRASESLDNYGIRFLT |
| | scFv | | WFQQKPGKAPKLLMYAASNQGSGVPSRFSGSGSGTEFT |
| | | | LTISSLQPDDFATYYCQQTKEVPWSFGQGTKVEVK |
| 41 | anti-B- | IMGT/2D | QVQLQQPGAELVKPGASVKMSCKASGYTFTSYN |
| | lymphocyte | structure-DB | MHWVKQTPGRGLEWIGAIYPGNGDTSYNQKFK |
| | antigen | card for INN | GKATLTADKSS |
| | CD20 | 7609 Shown is | STAYMQLSSLTSEDSAVYYCARSTYYGGDWYFN |
| | scFv | the VH region | VWGAGTTVTVSA [symbol] [symbol] QIVLSQ |
| | | linked to the | SPAILSASPGEKVTMTCRASSSVSYIHWFQQKPGS |
| | | VL region | SPKPWIYATSNLASGVPVRFSGSGSGTSYSLTISR |
| | | (linker region | VEAEDAATYYCQQWTSNPPTFGGGTKLEIK |
| | | show in | |
| | | italics and | |
| | | underlined) | |
| 56 & | anti- | | Heavy chain/Chaine lourde/Cadena pesada |

| | | | |
|---|---|---|---|
| 57 | CD123 | EVQLVQSGAE VKKPGESLKI SCKGSGYSFT DYYMKWARQM PGKGLEWMGD | 50 |
| | (talacotuzumab) | IIPSNGATFY NQKPKGQVTI SADKSISTTY LQWSSLKASD TAMYYCARSH | 100 |
| | | LLRASWFAYW GQGTMVTVSS ASTKGPSVFP LAPSSKSTSG GTAALGCLVK | 180 |
| | | DYFPEPVTVS WNSGALTSGV HTPDAVLQSS GLYSLSSVVT VPSSSLGTQT | 200 |
| | | YICNVNHKPS NTKVDKKVEP KSCDKTHTCP PCPAPELLGG PDVFLFPPEP | 250 |
| | | KDTLMISRTP EVTCVVVDVS SEDPEVQFSW YVDGVEVHNA KTKPRESQFN | 300 |
| | | STFRVVSVLT VVHQDWLNGH EYKCKVSNKA LPAPEEKTIS KTKGQPREPQ | 350 |
| | | VYTLPPSVLT MTKNQVSLTC LVKGFYPSDI AVEWESNGQP ENNYKTTFPM | 400 |
| | | LDSDGSFFLY SKLTVDKSRW QQGNVPSCSV MHEALHNBYT QKSLSLSPGE | 450 |
| | | Light chain/Chaine legere/Cadena ligen | |
| | | DIVMTQSPDS LAVSLGERAT INCESEQSLL NSGNQKNYLT WYQQKPGQPP | 50 |
| | | KPLIYWASTE ESGVPDRFSG SGSGTDFTLT ISSLQASDVA VYYCQNDYSY | 100 |
| | | PYTPGQGTKL EIKRTVAAPS VPISPPSDEQ LKSGTASVVC LLNNSYPREA | 150 |
| | | KVQWKVDNAL QSGNSQESVT EQDSKDSTYS LSSTLTLSKA DYEKHKVYAC | 200 |
| | | EVTRQGLSSP VTKSFNPGEC | 220 |

| | | |
|---|---|---|
| 58 & | Anti- | VH: |
| 59 | CD47 | QVQLVQSGAEVKKPGASVKVSCKASGYTFT |
| | (Hu5F9- | CDR1 CDR2 |
| | G4) | N̅Y̅N̅M̅H̅WVRQAPGQRLEWMGT̅I̅Y̅P̅G̅N̅D̅D̅T̅S̅Y̅ |
| | | N̅Q̅K̅F̅K̅D̅RVTITADTSASTAYMELSSLRSED |
| | | CDR3 |
| | | TAVYYCARG̅G̅Y̅R̅A̅M̅D̅Y̅WGQGTLVTVSS |
| | | VL: |
| | | CDR1 |
| | | DIVMTQSPLSLPVTPGEPASISCR̅S̅S̅Q̅S̅I̅V̅ |
| | | CDR2 |
| | | Y̅S̅N̅G̅N̅T̅Y̅L̅G̅WYLQKPGQSPQLLIYK̅V̅S̅N̅R̅F̅ |
| | | S̅GVPDRFSGSGSGTDFTLKISRVEAEDVGV |
| | | CDR3 |
| | | YYCF̅Q̅G̅S̅H̅V̅P̅Y̅T̅FGQGTKLEIK |
| 60 & | Anti- | VH: |
| 61 | BCMA | QVQLVQSGAEVKKPGSSVKVSCKASGGTFSNYWMHWVRQAPGQGLEWMGATYRGHSDTYYNQR |
| | | FKGRVHTADKSTSTAYMELSSLRSEDTAVYYCARGAIYDGYDVLDNWGQGTLVTVSS |
| | | VL(31) |
| | | DIQMTQSPSSLSASVGDRVTITCSASQDISNYLNWYQQKPGKAPKLWYYTSNLHSGVPSRFSGSGS |
| | | GTDFTLTISSLQPEDFATYYCQQYRKLPWTFGQGTKLEIKH |
| 104 | Anti- | EVQLLESGGGLVQPGGSLRLSCAVSGFTFNSFAM |
| | CD38 | SWVRQAPGKGLEWVSAISGSGGGTYYADSVKG |
| | (linker is | RFTISRDNSKNTLYLQMNSLRAEDTAVYFCAKD |
| | underlined) | KILWFGEPVFDYWGQGTLVTVSSGGGGSGGGGS |
| | | GGGGSEIVLTQSPATLSLSPGERATLSCRASQSVS |
| | | SYLAWYQQKPGQAPRLLIYDASNRATGIPARFSG |
| | | SGSGTDFTLTISSLEPEDFAVYYCQQRSNWPPTFG |
| | | QGTKVEIK |

TABLE 7A
Exemplary amino acid sequences and activities of
glutaminase molecules from various species.

| SEQ | | |
|---|---|---|
| ID | Sequence name and | |
| NO: | source | Amino acid sequence |
| 105 | Homo sapiens | MMRLRGSGML RDLLLRSPAG VSATLRRAQP |
| | Glutaminase kidney | LVTLCRRPRG GGRPAAGPAA |
| | isoform, | AARLHPWWGG GGWPAEPLAR GLSSSPSEIL |
| | mitochondrial | QELGKGSTHP QPGVSPPAAP |
| | Uniprot: O94925 | AAPGPKDGPG ETDAFGNSEG KELVASGENK |
| | | IKQGLLPSLE DLLFYTIAEG |
| | | QEKIPVHKFI TALKSTGLRT SDPRLKECMD |
| | | MLRLTLQTTS DGVMLDKDLF |
| | | KKCVQSNIVL LTQAFRRKFV IPDFMSFTSH |
| | | IDELYESAKK QSGGKVADYI |
| | | PQLAKFSPDL WGVSVCTVDG QRHSTGDTKV |
| | | PFCLQSCVKP LKYAIAVNDL |
| | | GTEYVHRYVG KEPSGLRFNK LFLNEDDKPH |
| | | NPMVNAGAIV VTSLIKQGVN |
| | | NAEKFDYVMQ FLNKMAGNEY VGFSNATFQS |
| | | ERESGDRNFA IGYYLKEKKC |
| | | FPEGTDMVGI LDFYFQLCSI EVTCESASVM |
| | | AATLANGGFC PITGERVLSP |
| | | EAVRNTLSLM HSCGMYDFSG QFAFHVGLPA |
| | | KSGVAGGILL VVPNVMGMMC |
| | | WSPPLDKMGN SVKGIHFCHD LVSLCNFHNY |
| | | DNLRHFAKKL DPRREGGDQR |
| | | VKSVINLLFA AYTGDVSALR RFALSAMDME |
| | | QRDYDSRTAL HVAAAEGHVE |
| | | VVKFLLEACK VNPFPKDRWN NTPMDEALHF |
| | | GHHDVFKILQ EYQVQYTPQG |
| | | DSDNGKENQT VHKNLDGLL |
| 106 | Bacillus subtilis | MLTIGVLGLQ GAVREHIHAI EACGAAGLVV |
| | Pyridoxal 5′- | KRPEQLNEVD GLILPGGEST |
| | phosphate synthase | TMRRLIDTYQ FMEPLREFAA QGKPMFGTCA |
| | subunit PdxT | GLIILAKEIA GSDNPHLGLL |
| | Uniprot: P37528 | NVVVERNSFG RQVDSFEADL TIKGLDEPFT |
| | | GVFIRAPHIL EAGENVEVLS |
| | | EHNGRIVAAK QGQFLGCSFH PELTEDHRVT |
| | | QLFVEMVEEY KQKALV |
| 107 | Escherichia coli | MLDANKLQQA VDQAYTQFHS LNGGQNADYI |
| | Glutaminase 1 | PFLANVPGQL AAVAIVTCDG |
| | Uniprot: P77454 | NVYSAGDSDY RFALESISKV CTLALALEDV |
| | | GPQAVQDKIG ADPTGLPFNS |
| | | VIALELHGGK PLSPLVNAGA IATTSLINAE |
| | | NVEQRWQRIL HIQQQLAGEQ |
| | | VALSDEVNQS EQTTNFHNRA IAWLLYSAGY |
| | | LYCDAMEACD VYTRQCSTLL |
| | | NTIELATLGA TLAAGGVNPL THKRVLQADN |
| | | VPYILAEMMM EGLYGRSGDW |
| | | AYRVGLPGKS GVGGGILAVV PGVMGIAAFS |
| | | PPLDEDGNSV RGQKMVASVA |
| | | KQLGYNVFKG |
| 108 | Mus musculus | MRSMRALQNA LSRAGSHGRR GGWGHPSRGP |
| | Glutaminase liver | LLGRGVRYYL GEAAAQGRGT |
| | isoform, | PHSHQPQHSD HDASHSGMLP RLGDLLFYTI |
| | mitochondrial | AEGQERIPIH KFTTALKATG |
| | Uniprot: Q571F8 | LQTSDPRLQD CMSKMQRMVQ ESSSGGLLDR |
| | | ELFQKCVSSN IVLLTQAFRK |
| | | KFVIPDFEEF TGHVDRIFED AKEPTGGKVA |
| | | AYIPHLAKSN PDLWGVSLCT |
| | | VDGQRHSVGH TKIPFCLQSC VKPLTYAISV |
| | | STLGTDYVHK FVGKEPSGLR |
| | | YNKLSLNEEG IPHNPMVNAG AIVVSSLIKM |
| | | DCNKAEKFDF VLQYLNKMAG |
| | | NEFMGFSNAT FQSEKETGDR NYAIGYYLKE |
| | | KKCFPKGVDM MAALDLYFQL |
| | | CSVEVTCESG SVMAATLANG GICPITGESV |
| | | LSAEAVRNTL SLMHSCGMYD |
| | | FSGQFAFHVG LPAKSAVSGA ILLVVPNVMG |
| | | MMCLSPPLDK LGNSQRGINF |
| | | CQKLVSLFNF HNYDNLRHCA RKLDPRREGG |
| | | EVRNKTVVNL LFAAYSGDVS |
| | | ALRRFALSAM DMEQKDYDSR TALHVAAAEG |
| | | HIEVVKFLIE ACKVNPFVKD |
| | | RWGNIPLDDA VQFNHLEVVK LLQDYHDSYL |
| | | LSETQAEAAA ETLSKENLES |
| | | MV |
| 109 | Bacillus subtilis | MKFAVIVLPG SNCDIDMYHA VKDELGHEVE |
| | Phosphoribosylformylglycinamidine | YVWHEETSLD GFDGVLIPGG |
| | synthase subunit | FSYGDYLRCG AIARFANIMP AVKQAAAEGK |
| | PurQ | PVLGVCNGFQ ILQELGLLPG |
| | Uniprot: P12041 | AMRRNKDLKF ICRPVELIVQ NDETLFTASY |
| | | EKGESITIPV AHGEGNFYCD |
| | | DETLATLKEN NQIAFTYGSN INGSVSDIAG |
| | | VVNEKGNVLG MMPHPERAVD |
| | | ELLGSADGLK LFQSIVKNWR ETHVTTA |
| 110 | Aspergillus oryzae | MMHFLSFCLS VASLVSYAGA ASTFSPARPP |
| | Glutaminase A | ALPLAVKSPY LSTWLSAGTD |
| | Uniprot: Q2U4L7 | GGNGGYLAGQ WPTFWFGQVT GWAGQIRVDN |
| | | STYTWMGAIP NTPTVNQTSF |
| | | EYTSTSSVFT MRVGDMVEMK VKFLSPITPD |
| | | DLRRQSLVFS YLDVDVESID |
| | | GKAHDIQVYA DISAEWASGD RNAIAQWDYG |
| | | VTDDGVAYHK VYRQTQLLFS |
| | | ENTEQAEWGE WYWATDDQDG LSYQSGPDVD |
| | | VRGAFAKNGK LANSDDKNYR |
| | | AISTNWPVFA FSRDLGSVKT SAGTLFSIGL |
| | | AQDSAIQYSG KPEGTTVMPS |
| | | LWKSYFSTAT AALEFFHHDY AAAAALSKDL |
| | | DDRISKDSID AAGQDYLTIT |
| | | SLTVRQVFAA VQLTGTPEDP YIFMKEISSN |
| | | GNMNTVDVIF PAHPIFLYTN |
| | | PELLKLILKP IYEIQENGKY PNTYAMHDIG |
| | | THYPNATGHP KGDDEKMPLE |
| | | ECGNMVIMAL AYAQKAKDND YLSQHYPILN |
| | | KWTTYLVEDS IYPANQISTD |
| | | DFAGSLANQT NLALKGIIGI QAMAVISNTT |
| | | GHPDDASNHS SIAKDYIARW |
| | | QTLGVAHDAN PPHTTLSYGA NETHGLLYNL |
| | | YADRELGLNL VPQSVYDMQN |
| | | TFYPTVKEKY GVPLDTRHVY TKADWELFTA |
| | | AVASESVRDM FHQALATWIN |
| | | ETPTNRAFTD LYDTQTGNYP AGITFIARPV |
| | | MGGAFALLIL |
| 111 | Thermotoga | MKPRACVVVY PGSNCDRDAY HALEINGFEP |
| | maritima | SYVGLDDKLD DYELIILPGG |
| | Phosphoribosylformylglycinamidine | FSYGDYLRPG AVAAREKIAF EIAKAAERGK |
| | synthase subunit | LIMGICNGFQ ILIEMGLLKG |
| | PurQ | ALLQNSSGKF ICKWVDLIVE NNDTPFTNAF |
| | Uniprot: Q9X0X2 | EKGEKIRIPI AHGFGRYVKI |
| | | DDVNVVLRYV KDVNGSDERI AGVLNESGNV |
| | | FGLMPHPERA VEELIGGEDG |
| | | KKVFQSILNY LKR |
| 112 | Acinetobacter | KNNVVIVATG GTIAGAGASS TNSATYSAAK |
| | glutaminasificans | VPVDALIKAV PQVNDLANIT |
| | Glutaminase- | GIQALQVASE SITDKELLSL ARQVNDLVKK |
| | asparaginase | PSVNGVVITH GTDTMEETAF |
| | Uniprot: P10172 | FLNLVVHTDK PIVLVGSMRP STALSADGPL |
| | | NLYSAVALAS SNEAKNKGVM |
| | | VLMNDSIFAA RDVTKGINIH THAFVSQWGA |
| | | LGTLVEGKPY WFRSSVKKHT |
| | | NNSEFNIEKI QGDALPGVQI VYGSDNMMPD |
| | | AYQAFAKAGV KAIIHAGTGN |
| | | GSMANYLVPE VRKLHDEQGL QIVRSSRVAQ |
| | | GFVLRNAEQP DDKYGWIAAH |
| | | DLNPQKARLL MALALTKTND AKEIQNMFWN Y |
| 113 | Pseudomonas putida | MNAALKTFAP SALALLLILP SSASAKEAET |
| | Glutaminase- | QQKLANVVIL ATGGTIAGAG |
| | asparaginase | ASAANSATYQ AAKLGVDKLI AGVPELADIA |
| | Uniprot: Q88K39 | NVRGEQVMQI ASESISNDDL |
| | | LKLGKRVAEL AESKDVDGIV ITHGTDTLEE |
| | | TAFFLNLVEK TDKPIVVVGS |
| | | MRPGTAMSAD GMLNLYNAVA VASDKQSRGK |
| | | GVLVTMNDEI QSGRDVSKAV |
| | | NIKTEAFKSA WGPMGMVVEG KSYWFRLPAK |
| | | RHTVNSEFDI KQISSLPQVD |
| | | IAYGYGNVTD TAYKALAQNG AKALIHAGTG |
| | | NGSVSSRVVP ALQELRKNGV |
| | | QIIRSSHVNQ GGFVLRNAEQ PDDKNDWVVA |
| | | HDLNPQKARI LAMVAMTKTQ |
| | | DSKELQRIFW EY |

### EXAMPLES

### Example 1: Erythroid Cells are Genetically Engineered to Express an Amino Acid Degradative Enzyme

### Results

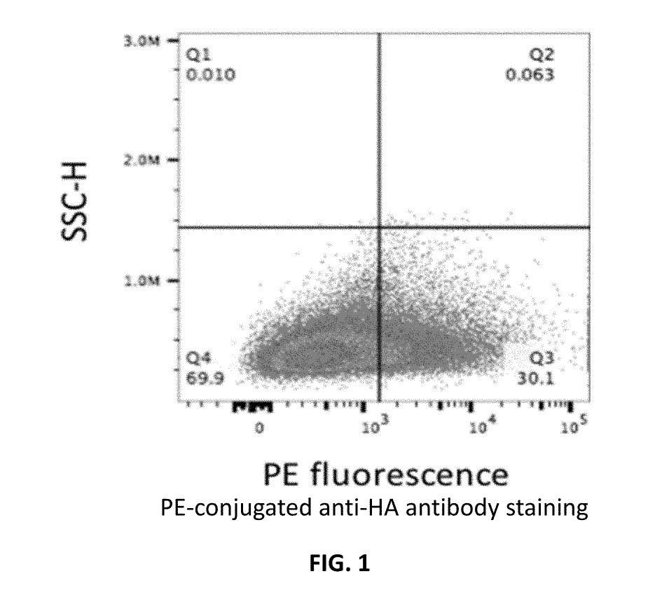

Erythroid cells were electroporated to express a fusion protein comprising the Kell transmembrane domain fused to an amino acid degradative enzyme, Erwinia asparaginase (Kell-ErwASNase) (SEQ ID NO: 1) on the surface, and having an HA tag, as described in the “Methods” section below. The electroporated cells were incubated with PE-conjugated anti-HA tag antibody and analyzed via flow cytometry. A gate was set based on stained non-electroporated cells. As shown in FIG. 1, over 30% of cells in the population had asparaginase on their surface at a level above this cutoff.

### Methods

### Expansion and Differentiation of Erythroid Cells

Human CD34+ cells derived from mobilized peripheral blood cells from normal human donors were purchased frozen from AllCells Inc. The expansion/differentiation procedure comprised 3 stages. In the first stage, thawed CD34+ erythroid precursors were cultured in Iscove's MDM medium comprising recombinant human insulin, human transferrin, recombinant human recombinant human stem cell factor, and recombinant human interleukin 3. In the second stage, erythroid cells were cultured in Iscove's MDM medium supplemented with bovine serum albumin, recombinant human insulin, human transferrin, human recombinant stem cell factor, human recombinant erythropoietin, and L-glutamine. In the third stage, erythroid cells were cultured in Iscove's MDM medium supplemented with human transferrin, recombinant human insulin, human recombinant erythropoietin, and heparin. The cultures were maintained at 37° C. in 5% CO₂ incubator.

### Electroporation of Erythroid Precursor Cells

Erythroid cells were electroporated together with mRNA encoding Kell-ErwASNase via Nucleofector electroporator. Erythroid cells were stained with PE-conjugated anti-HA antibody and analyzed via flow cytometry 24 hours after electroporation.

### Example 2: Erythroid Cells are Genetically Engineered to Express a Cell Targeting Moiety, which Binds its Ligand

Erythroid cells were transduced to express a fusion protein comprising a GPA transmembrane domain fused to a cell targeting moiety, an anti-CD33 scFv (HA-αCD33scFv-GPA) (SEQ ID NO: 2). Cell culture and transduction was performed as described in “Methods” section below to yield erythroid cells expressing an anti-CD33 antibody molecule on the surface, anchored with a GPA transmembrane domain.

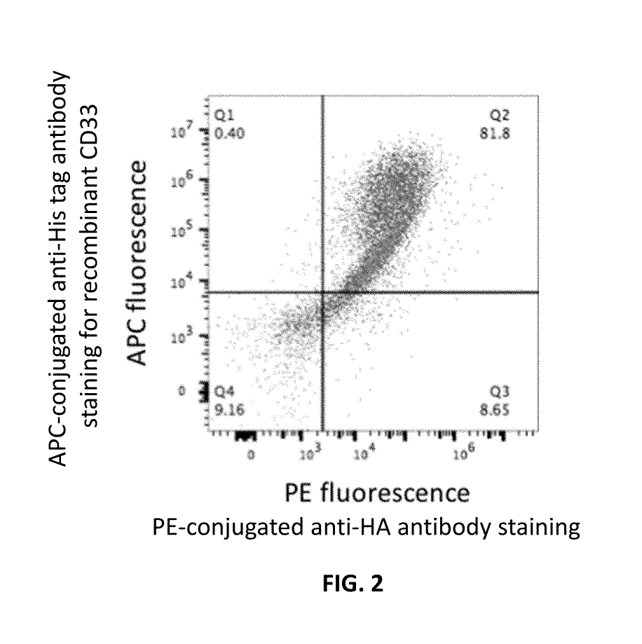

Because the construct contains an HA tag, the exogenous protein can be detected with a labeled anti-HA antibody. Transduced cells were incubated with recombinant CD33-HisTag to detect binding of the exogenous HA-αCD33scFv-GPA to its ligand. The cells were subsequently co-incubated with an APC-conjugated anti-HisTag antibody to detect the bound CD33-HisTag, and PE-conjugated anti-HA tag antibody to detect the HA tag in the exogenous HA-αCD33scFv-GPA. The cells were analyzed by flow cytometry for APC fluorescence and PE fluorescence. A gate was set based on stained untransduced cells. As shown in FIG. 2, over 80% of cells expressed αCD33scFv-GPA that bound to recombinant CD33.

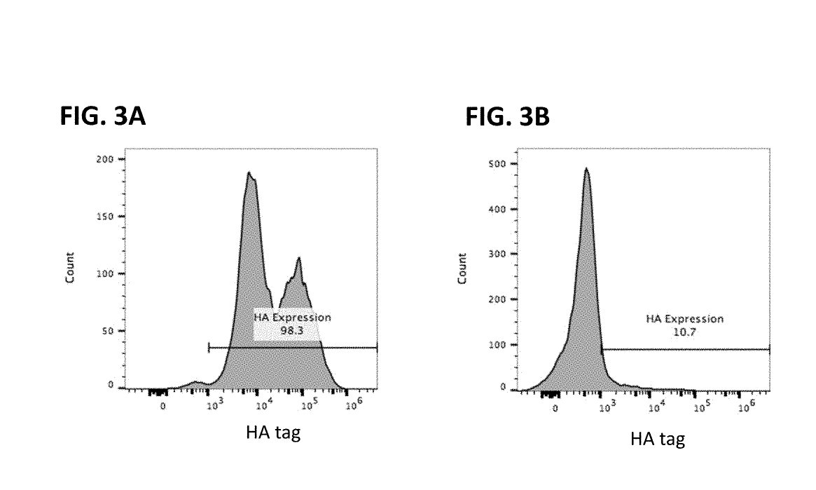

Erythroid cells having αCD33scFv on their surface were also tested for the ability to bind AML cells expressing CD33. The CD33 expressing cells used in this experiment were MV-4-11 human AML cells. MV4-11 cells were labeled with carboxyfluorescein succinimidyl ester (CFSE). Subsequently the labeled MV4-11 cells were incubated with αCD33scFv expressing erythroid cells, stained with fluorescently labeled antibody against HA tag on exogenously expressed αCD33scFv construct and analyzed via flow cytometry. Binding of the engineered erythroid cells to the CD33 expressing cells was detected by analyzing HA tag positive erythroid cells within CFSE-positive cell population (MV4-11 cells) (FIG. 3A). Negative control HA-GPA expressing erythroid cells showed minimal binding to CD33-expressing AML cells (FIG. 3B). 10.7% of HA-GPA erythroid cells were positive for binding to MV-4-11, whereas 98.3% of αCD33scFv erythroid cells were positive for binding to MV4-11.

### Methods

Production of Lentiviral Vector

The gene encoding the HA-αCD33scFv-GPA fusion protein was cloned into the multiple cloning site of lentivirus vector pCDH with the MSCV promoter sequence from System Biosciences. Lentivirus was produced in 293T cells by transfecting the cells with pPACKH1 (System Biosciences) and pCDH lentivirus vector containing HA-αCD33scFv-GPA gene. Cells were then placed in fresh culturing medium. The virus supernatant was collected 48 hours post-medium change by centrifugation at 1,500 rpm for 5 minutes. The supernatant was collected and frozen in aliquots at −80° C.

### Expansion and Differentiation of Erythroid Cells

Human CD34+ cells derived from mobilized peripheral blood cells from normal human donors were purchased frozen from AllCells Inc. The expansion/differentiation procedure comprised 3 stages. In the first stage, thawed CD34+ erythroid precursors were cultured in Iscove's MDM medium comprising recombinant human insulin, human transferrin, recombinant human recombinant human stem cell factor, and recombinant human interleukin 3. In the second stage, erythroid cells were cultured in Iscove's MDM medium supplemented with bovine serum albumin, recombinant human insulin, human transferrin, human recombinant stem cell factor, human recombinant erythropoietin, and L-glutamine. In the third stage, erythroid cells were cultured in Iscove's MDM medium supplemented with human transferrin, recombinant human insulin, human recombinant erythropoietin, and heparin. The cultures were maintained at 37° C. in 5% CO₂ incubator.

### Transduction of Erythroid Precursor Cells

Erythroid precursor cells were transduced during step 1 of the culture process described above. Erythroid cells in culturing medium were combined with lentiviral supernatant and polybrene. Infection was achieved by spinoculation, spinning the plate at 2000 rpm for 90 minutes at room temperature. After spinoculation, the cells were incubated at 37° C. overnight.

### Example 3: Erythroid Cells Expressing an Amino Acid Degradative Enzyme can Degrade its Amino Acid Substrate

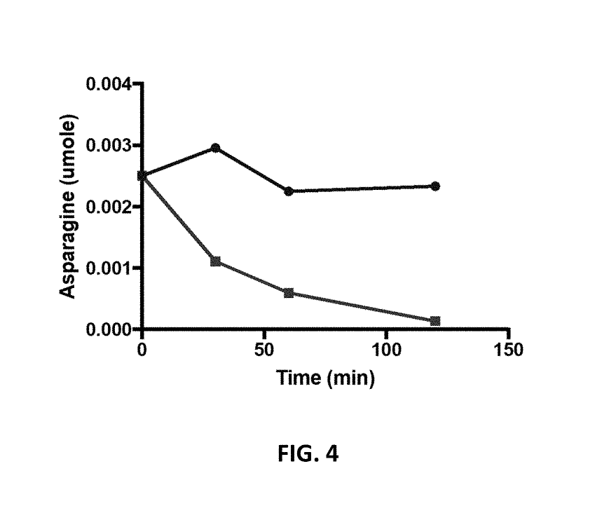

Erythroid cells were genetically engineered to express a fusion protein comprising the Kell transmembrane domain fused to an amino acid degradative enzyme, Erwinia asparaginase, as described in Example 1. 1e6 erythroid cells expressing Kell-ErwASNase anchored with a Kell transmembrane domain on the surface were incubated with 100 uM asparagine in 100 uL of PBS. 20 uL aliquots were taken at the indicated time points, cells were eliminated via centrifugation, and the supernatant was analyzed for asparagine concentration via mass spectrometry. FIG. 4 shows that non-electroporated control cells (circles) do not significantly reduce asparagine levels in this assay, whereas cells expressing the asparaginase molecule (squares) reduce asparagine levels by 94.6% over the course of the assay.

### Example 4. Generation and In Vitro Validation of Enucleated Erythroid Cells Expressing an Amino Acid Degradative Enzyme and a Cell Targeting Moiety

### Production of Lentiviral Vector

The genes for SMIM-ErwASNase and αCD33scFv-GPA are constructed. Genes encoding the fusion proteins are cloned into the multiple cloning site of lentivirus vector pCDH with the MSCV promoter sequence from System Biosciences, such that one vector comprises the genes for both exogenous proteins. Lentivirus is produced in 293T cells by transfecting the cells with pPACKH1 (System Biosciences) and pCDH lentivirus vector containing genes for SMIM-ErwASNase and αCD33scFv-GPA. Cells are placed in fresh culturing medium. The virus supernatant is collected 48 hours post-medium change by centrifugation at 1,500 rpm for 5 minutes. The supernatant is collected and frozen in aliquots at −80° C.

### Transduction of Erythroid Precursor Cells

Expansion and differentiation of erythroid cells is performed according to Example 1. Erythroid precursor cells are transduced during step 1 of the culture process. Erythroid cells in culturing medium are combined with lentiviral supernatant and polybrene. Infection is achieved by spinoculation, spinning the plate at 2000 rpm for 90 minutes at room temperature. After spinoculation, the cells are incubated at 37° C. overnight.

### Functional Validation Assay

The enucleated erythroid cells expressing the membrane-anchored amino acid degradative enzyme SMIM-ErwASNase and the membrane-anchored cell targeting moiety anti-CD33scFv-GPA are monitored for asparaginase activity according to an assay of Example 3, and binding of CD33 to the anti-CD33 antibody is measured according to an assay of Example 2. CD33 activity can also be monitored by binding of the anti-CD33 antibody expressed by the enucleated erythroid cells to a CD33 expressing leukemia cell line, such as MV4-11, as described in Example 2.

### Example 5. Activity of Enucleated Erythroid Cells Expressing an Amino Acid Degradative Enzyme and a Cell Targeting Moiety in a Mouse AML Model

The enucleated erythroid cells comprising an asparaginase molecule and an anti-CD33 antibody molecule are made as described in the previous example.

To assay the cells in a mouse AML model, the enucleated erythroid cells are injected intravenously into a xenograft AML model mice bearing leukemia tumors. In some embodiments, administration of the enucleated erythroid cells expressing an asparaginase molecule and an anti-CD33 antibody molecule will lead to one or both of inhibition of tumor cell engraftment or reduction of the tumor size, compared to unmodified control cells.

### Example 6. In Vivo Activity and Tolerance of Enucleated Erythroid Cells Comprising an Amino Acid Degradative Enzyme

Study #1

Recombinant E. coli asparaginase was conjugated to mouse red blood cells (mRBCs) to make mRBC-ASNase cells. The mRBC-ASNase cells were tested for their ability to deplete asparagine levels in plasma in vivo in mice. The tolerogenic properties of the cells were also tested in mice.

Mice were injected with control mRBC and mRBCs labeled to 3 different degrees with E. coli asparaginase as depicted in Table 8 below. All mRBCs were additionally labeled with the fluorescent molecule Cy5, to determine the pharmacokinetic properties of the mRBCs following injection. Blood samples were then taken at various times after injection to determine the levels of asparagine in mouse plasma as well as the levels of labeled mRBCs.

TABLE 8
Set-up for mouse study with asparaginase labeled mRBCs

| | | | Number | | Asparaginase | Total units of |
|---|---|---|---|---|---|---|
| | | Number | of | | activity of | asparaginase |
| | Recipient | of | mRBCs | | labeled mRBCs | injected per |
| | mice | animals | injected | Description | (units/cell) | mouse |
| Group 1 | C57BL/6J | 4 | 1e9 | Control mRBCs | — | — |
| | | | | labeled with Cy5 | | |
| | | | | only | | |
| Group 2 | C57BL/6J | 4 | 1e9 | 1e9 cells | ~5.2e−11 | ~0.052 |
| | | | | conjugated to high | | |
| | | | | level of | | |
| | | | | asparaginase and | | |
| | | | | labeled with Cy5 | | |
| Group 3 | C57BL/6J | 4 | 1e9 | 1e9 cells | ~2.2e−11 | ~0.022 |
| | | | | conjugated to an | | |
| | | | | intermediate level | | |
| | | | | of asparaginase and | | |
| | | | | labeled with Cy5 | | |
| Group 4 | C57BL/6J | 4 | 1e9 | 1e9 cells | ~8.2e−12 | ~0.0082 |
| | | | | conjugated to a low | | |
| | | | | level of | | |
| | | | | asparaginase and | | |
| | | | | labeled with Cy5 | | |

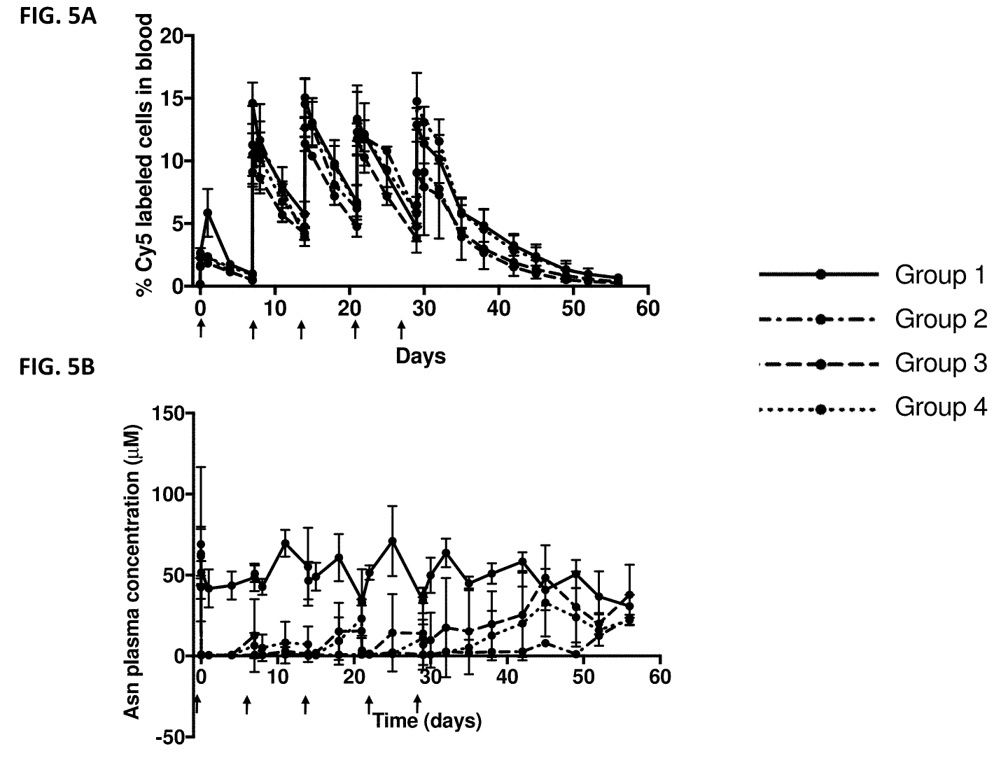

Upon injection of asparaginase-conjugated mRBCs (all 3 asparaginase treatment groups) the plasma asparagine levels were depleted to close to 0 within 30 minutes. Mice were injected with control and asparaginase-labeled mRBCs once a week for 5 weeks (FIG. 5B). Plasma asparagine levels remained significantly lower in asparaginase treatment groups compared to the control group for almost the entire study period until about 12 days post final fifth dose. The highest asparaginase dose (group 2) resulted in the most consistent and strongest depletion of plasma asparagine levels. The pharmacokinetic profile of the asparaginase-labeled mRBCs was essentially the same as that of control mRBCs (FIG. 5A). The pharmacokinetic profile for the asparaginase-labeled mRBCs also did not change with repeated dosing, indicating a lack of immunogenic response to the asparaginase-labeled mRBCs for all three levels of asparaginase, as detectable by this assay.

Study #2

Recombinant Erwinia chrysanthemi asparaginase was conjugated to mouse red blood cells (mRBCs) (referred to in this example as mRBC-ASNase). The mRBC-ASNase cells were tested for their tolerogenic or immunogenic properties in vivo in immune-competent C57BL/6J mice. The ability of the cells to induce an anti-ASNase antibody response in mice was also tested.

Mice were injected with control mRBCs (mRBCs solely labeled with Cy5) or mRBC-ASNase labeled with either ˜500, ˜2500, ˜10,000, or ˜40,000 Erwinia chrysanthemi asparaginase molecules per cell. All mRBCs were additionally labeled with the fluorescent molecule Cy5, to determine the pharmacokinetic properties of the mRBCs following injection. As an additional control, a group of mice was dosed with 15 μg of recombinant Erwinia chrysanthemi protein. 3 doses were given at days 0, 28 and 57 as described in Table 9. Blood samples were taken at various days to determine the levels of anti-asparaginase antibodies in the mouse plasma as well as the pharmacokinetic profile of the cells.

TABLE 9
Set-up for mouse study with Erwinia
chrysanthemi asparaginase labeled mRBCs

| | | | Total | Total | Total |
|---|---|---|---|---|---|
| | | | units of | units of | units of |
| | | | aspara- | aspara- | aspara- |
| | Number | | ginase | ginase | ginase |
| | of | | injected | injected | injected |
| | mRBCs | | per mouse: | per mouse: | per mouse: |
| | injected | Description | dose #1 | dose #2 | dose #3 |
| Group | 1 × 10⁹ | Control | — | — | — |
| 1 | | mRBCs | | | |
| | | labeled | | | |
| | | with Cy5 | | | |
| | | only | | | |
| Group | 1 × 10⁹ | mRBC- | 1.95 | 1.85 | 4.55 |
| 2 | | ASNase | | | |
| | | labeled | | | |
| | | with Cy5 | | | |
| | | and ~40,000 | | | |
| | | ASNase | | | |
| | | copies/cell | | | |
| Group | 1 × 10⁹ | mRBC- | 0.0893 | 0.124 | 0.304 |
| 3 | | ASNase | | | |
| | | labeled | | | |
| | | with Cy5 | | | |
| | | and ~10,000 | | | |
| | | ASNase | | | |
| | | copies/cell | | | |
| Group | 1 × 10⁹ | mRBC- | 0.0135 | 0.00265 | 0.0407 |
| 4 | | ASNase | | | |
| | | labeled | | | |
| | | with Cy5 | | | |
| | | and ~500 | | | |
| | | ASNase | | | |
| | | copies/cell | | | |

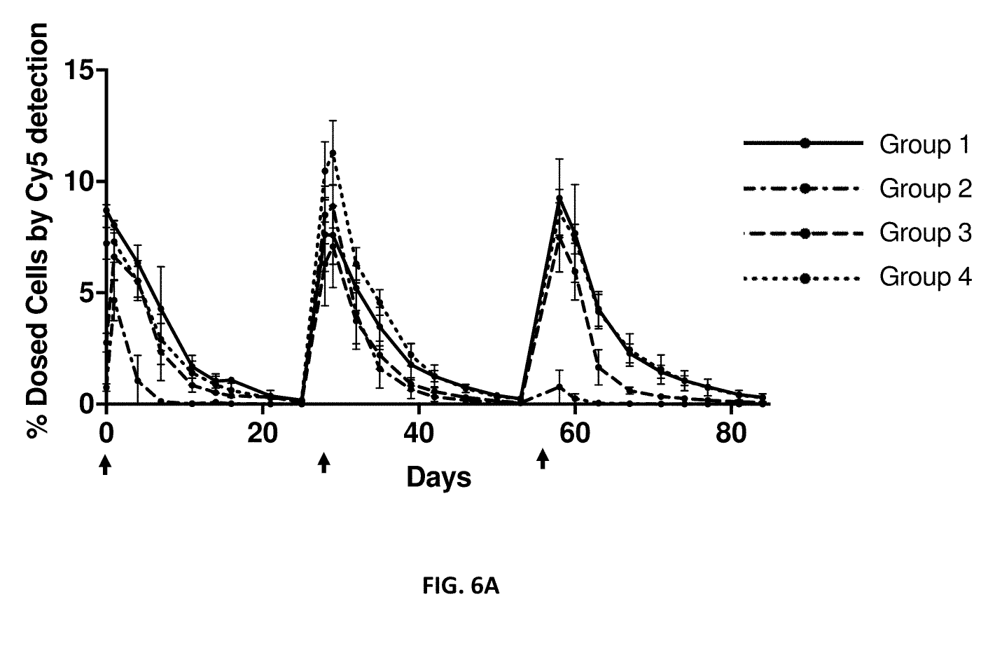

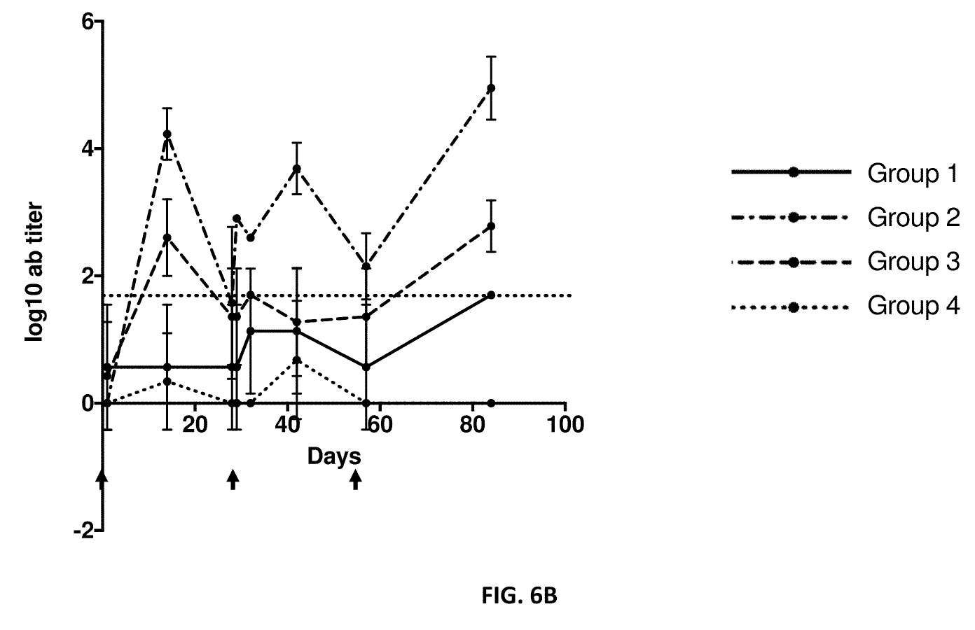

As shown in FIG. 6A, the pharmacokinetic profile of mRBC-ASNase labeled with either 500 (Group 4) or 10,000 (Group 3) asparaginase copies/cell was essentially identical to that of control mRBCs (Group 1) over all 3 doses; whereas the pharmacokinetic profile of mRBCs labeled with 40,000 asparaginase copies/cell (Group 2) showed rapid clearance of the cells upon administration of the 3^{rd} dose.

As shown in FIG. 6B, mice dosed with mRBC-ASNase labeled with either 500 (Group 4) or 10,000 (Group 3) asparaginase copies/cell did not exhibit significantly higher serum anti-asparaginase titers as compared to mice treated with control mRBCs (Group 1). In contrast, mice dosed with mRBC-ASNase labeled with 40,000 asparaginase copies/cell (Group 2) exhibited significantly higher serum anti-asparaginase titers as compared to mice treated with control mRBCs (Group 1).

Both data sets demonstrate the tolerogenic properties of mRBCs labeled with lower densities/degrees of recombinant Erwinia chrysanthemi asparaginase.

### Example 7. In Vivo Activity of Erythroid Cells Comprising the Asparagine and Glutamine Degradative Enzyme (Asparaginase/Glutaminase) from Pseudomonas 7A

To assess the in vivo activity of enucleated erythroid cells comprising an amino acid degradative enzyme on its surface, the following experiment was performed. Briefly, recombinant asparaginase/glutaminase from Pseudomonas 7A including a His tag (PseuGLNase) was conjugated to mouse red blood cells (mRBCs) (referred to in this example as mRBC-PseuGLNase). Suitable click conjugation methods are described, e.g., in International Application WO2018/151829, which is herein incorporated by reference in its entirety. The mRBC-PseuGLNase were tested for their ability to deplete plasma asparagine and glutamine levels in vivo in NOD SCID mice (Jackson Laboratory, Stock No. 001303). Mice were injected with control mRBCs (mRBCs solely labeled with Cy5) or mRBCs labeled with various doses of PseuGLNase and Cy5 according to the study design depicted in Table 10. All mRBCs were labeled with the fluorescent molecule Cy5 to determine the pharmacokinetic properties of the mRBCs following injection. Mice were administered one dose of cells. Blood samples were collected on various days to determine the pharmacokinetic profile of Cy5-labeled mRBCs (+/−PseuGLNase), as well as plasma asparagine and glutamine levels.

TABLE 10
Experimental set-up for in vivo mouse
study using mRBCs conjugated with
asparaginase/glutaminase from Pseudomonas 7A

| | | | | Total units of |
|---|---|---|---|---|
| | | | | asparaginase |
| | Number/ | Number | | activity |
| | type | of | Description | injected per |
| | of mice | mRBCs | of cell | mouse in |
| | injected | injected | injected | 1 dose |
| Group | 4 NOD | 5 × 10⁸ | Control mRBCs: | None |
| 1 | SCID | | mRBCs labeled | |
| | mice | | with Cy5 | |
| Group | 4 NOD | 5 × 10⁸ | mRBC- | 9.8 × 10⁻² |
| 2 | SCID | | PseuGLNase | |
| | mice | | | |
| Group | 4 NOD | 5 × 10⁸ | mRBC- | 1.8 × 10⁻² |
| 3 | SCID | | PseuGLNase | |
| | mice | | | |
| Group | 4 NOD | 5 × 10⁸ | mRBC- | 3 × 10⁻³ |
| 4 | SCID | | PseuGLNase | |
| | mice | | | |
| Group | 4 NOD | 5 × 10⁷ | mRBC- | 3 × 10⁻⁴ |
| 5 | SCID | | PseuGLNase | |
| | mice | | | |

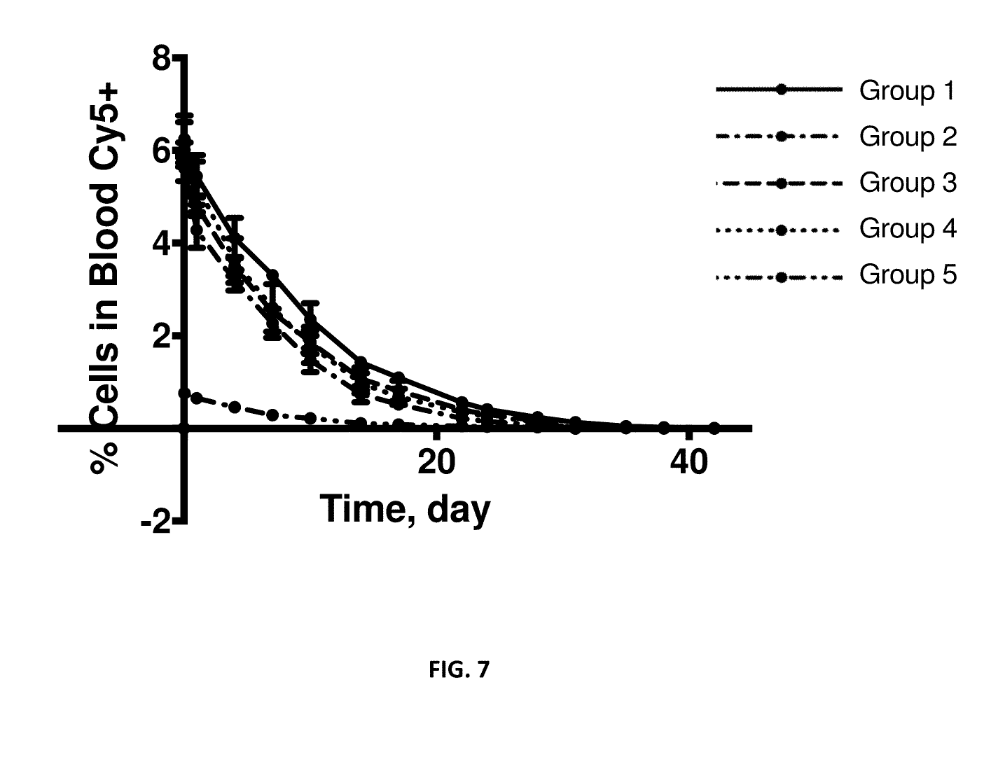

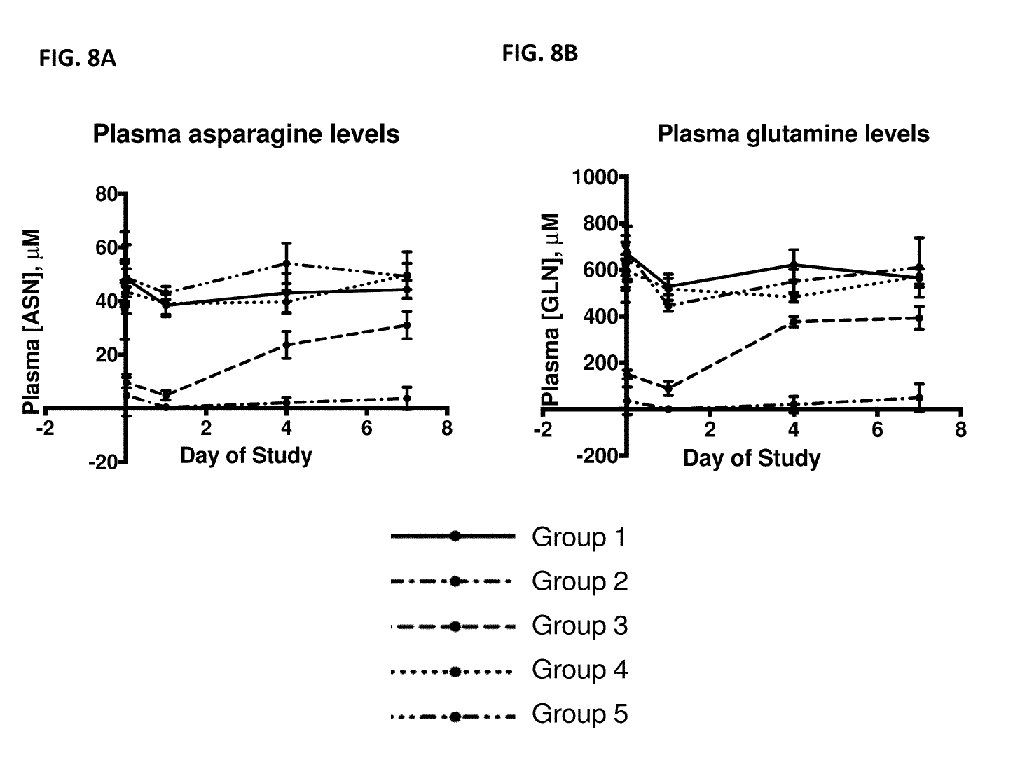

As shown in FIG. 7, no difference was observed in the pharmacokinetic profiles between the 5 groups of mice. Complete or almost complete depletion of both plasma asparagine (FIG. 8A) and glutamine (FIG. 8B) levels was observed for at least 7 days after mRBC-PseuGLNase were administered to Group 2 mice. Administration of mRBCs-PseuGLNase having lower asparaginase activity (Group 3) resulted in a reduction of plasma glutamine and asparagine levels for up to about 7 days.

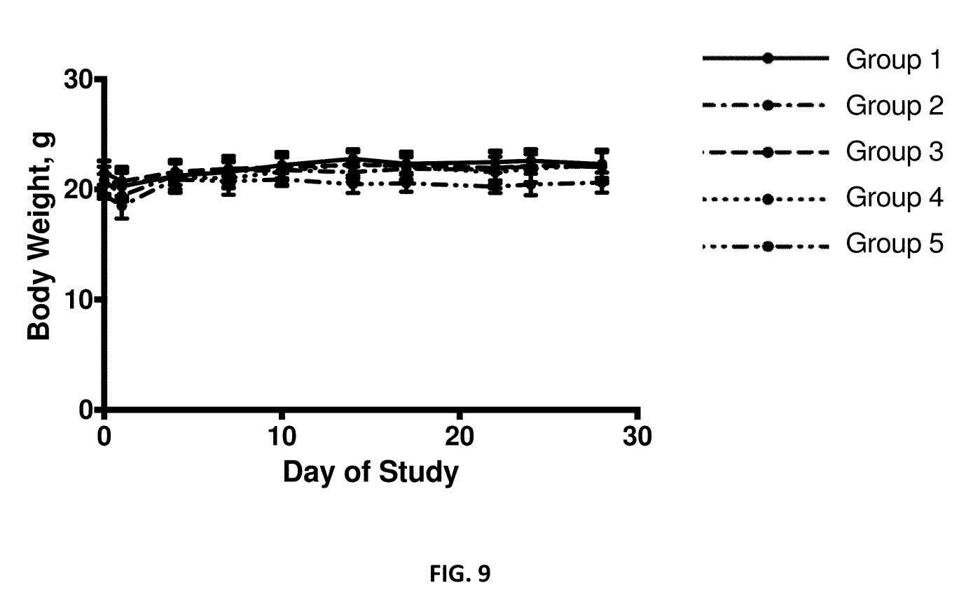

Surprisingly, although Group 2 mice exhibited depleted plasma asparagine and glutamine levels for about 7 days, no change in body weights was observed as compared to initial body weight or to control (Group 1) average body weight (FIG. 9). Further, survival in the treatment groups (including Group 2 mice), was 100%. Additionally, no adverse reactions to mRBC-PseuGLNase (e.g., decreased activity, rough coat, or hunched posture) were observed. This lack of toxicity was particularly surprising since many published mouse and human studies have documented that treatment with enzymes having glutaminase activity is highly toxic (see, e.g., Nguyen et al. (2018) Cancer Res. 78(6): 1549-1560; Warrell et al. (1982) Cancer Treat Rep. 66(7): 1479-1485; and Warrell et al. (1980) Cancer Res. 40(12): 4546-4551). Without wishing to be bound by any particular theory, administration of amino acid degradative enzymes, such as asparaginase and/or glutaminase, using enucleated cells as a vehicle appears to advantageously limit and/or reduce the toxicity of the enzyme.

### Example 8. In Vivo Activity of Enucleated Erythroid Cells Comprising PseuGLNase and Anti-CD33 scFv in a Disseminated MV4-11 AML Mouse Model

To assess the in vivo therapeutic activity of enucleated erythroid cells comprising a first polypeptide comprising the amino acid degradative enzyme PseuGLNase, and a second polypeptide comprising the cell targeting moiety anti-CD33 scFv, the following studies were performed using a human AML mouse model. Briefly, recombinant PseuGLNase was conjugated to mRBCs (referred to in this example as mRBC-PseuGLNase). A portion of the conjugated mRBC-PseuGLNase were also conjugated to varying copy number of recombinant anti-human CD33 scFv (amino acids 1-285 of SEQ ID NO: 55) (referred to in this example as mRBC-PseuGLNase/αCD33scFv). Additionally, recombinant asparaginase from E. coli (EcoliASNase; ABCAM, Cat. No. ab73439) was conjugated to mRBCs (referred to in this example as mRBC-EcoliASNase). Control mRBCs were solely labeled with Cy5, while mRBC-PseuGLNase, mRBC-PseuGLNase/αCD33scFv, and mRBC-EcoliASNase were also labeled with Cy5 for pharmacokinetic tracking.

Mice of a disseminated MV4-11 AML mouse model were developed by injecting NSG mice (NOD.Cg-Prkdc^{scid} IL2rg^{tm1Wjl}lSzJ; 005557; Jackson Laboratory) with 2×10⁶ human AML MV4-11 cells. Tumor load was tracked in peripheral blood by staining RBC-lysed whole blood from the mice with anti-human CD33 antibody and anti-human CD45 antibody to detect MV4-11 cells. Three weeks after MV4-11 cell implantation, MV4-11 cell tumor load in peripheral blood was about 0.5%-2% (percentage of cells staining for both CD33 and CD45 in the RBC-lysed whole blood). MV4-11 cell-engrafted mice were dosed once 24 days post-MV4-11 cell implantation with the conjugated mRBCs described above according to the experimental set-up provided in Table 11.

TABLE 11
Experimental set-up for in vivo disseminated
MV4-11 AML mouse model efficacy study

| | Number/ | Number | | Average total units of |
|---|---|---|---|---|
| | type | of | | asparaginase activity |
| | of mice | mRBCs | Description of | injected per |
| | injected | injected | cell injected | mouse in 1 dose |
| Group 1 | 10 NSG mice | 9 × 10⁸ | Control mRBCs: | None |
| | engrafted with | | mRBCs labeled with Cy5 | |
| | MV4-11 cells | | | |
| Group 2 | 10 NSG mice | 9 × 10⁸ | PseuGLNase and Cy5-labeled | 0.092 |
| | engrafted with | | mRBCs (mRBC- | |
| | MV4-11 cells | | PseuGLNase) | |
| Group 3 | 10 NSG mice | 9 × 10⁸ | EcoliASNase and Cy5- | 0.25 |
| | engrafted with | | labeled mRBCs (mRBC- | |
| | MV4-11 cells | | EcoliASNase) | |
| Group 4 | 10 NSG mice | 9 × 10⁸ | PseuGLNase, αCD33scFv | 0.054 |
| | engrafted with | | and Cy5-labeled mRBCs | |
| | MV4-11 cells | | (mRBC-PseuGLNase/ | |
| | | | αCD33scFv) | |
| Group 5 | 10 NSG mice | 9 × 10⁸ | PseuGLNase, αCD33scFv | 0.026 |
| | engrafted with | | and Cy5-labeled mRBCs | |
| | MV4-11 cells | | (mRBC-PseuGLNase/ | |
| | | | αCD33scFv) | |

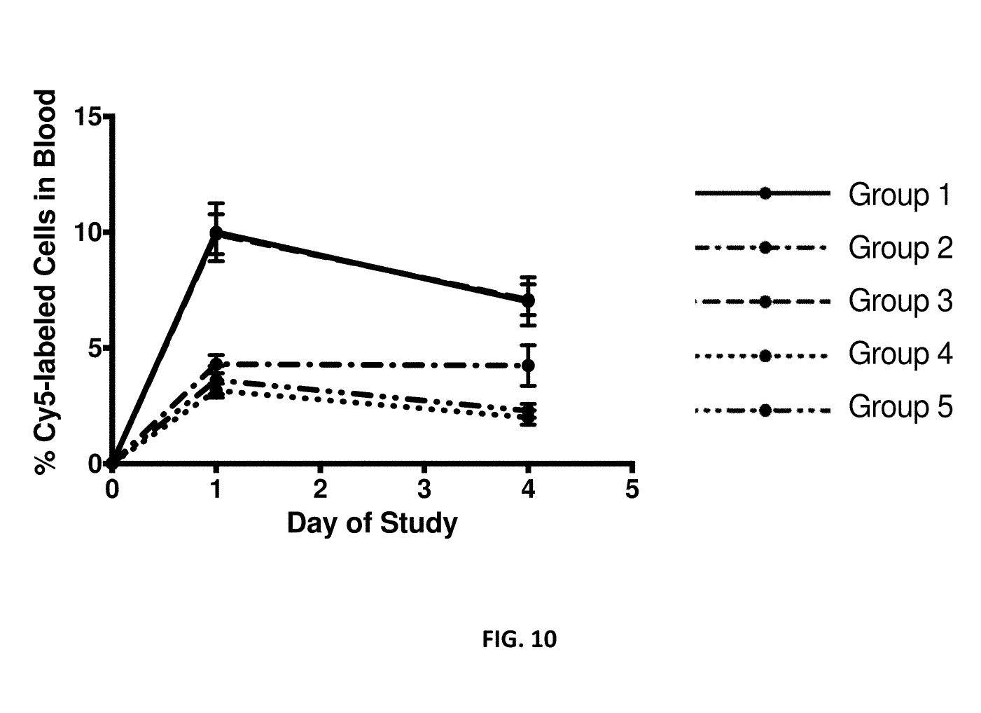

The pharmacokinetic profiles of the mRBCs in various groups was tracked by monitoring Cy5 fluorescence. As shown in FIG. 10, a sizable percentage of cells in the blood of mice in all groups were Cy5-labeled mRBCs.

The ability of mRBC-PseuGLNase (Group 2), mRBC-EcoliASNase (Group 3), mRBC-PseuGLNase/αCD33scFv (Groups 4 and 5) to induce MV4-11 cell death in vivo was evaluated by quantitating the total number of MV4-11 cells present in the whole blood of mice from each group. MV4-11 cells present in RBC-lysed whole blood from the mice was stained using anti-human CD33 and anti-human CD45 antibodies.

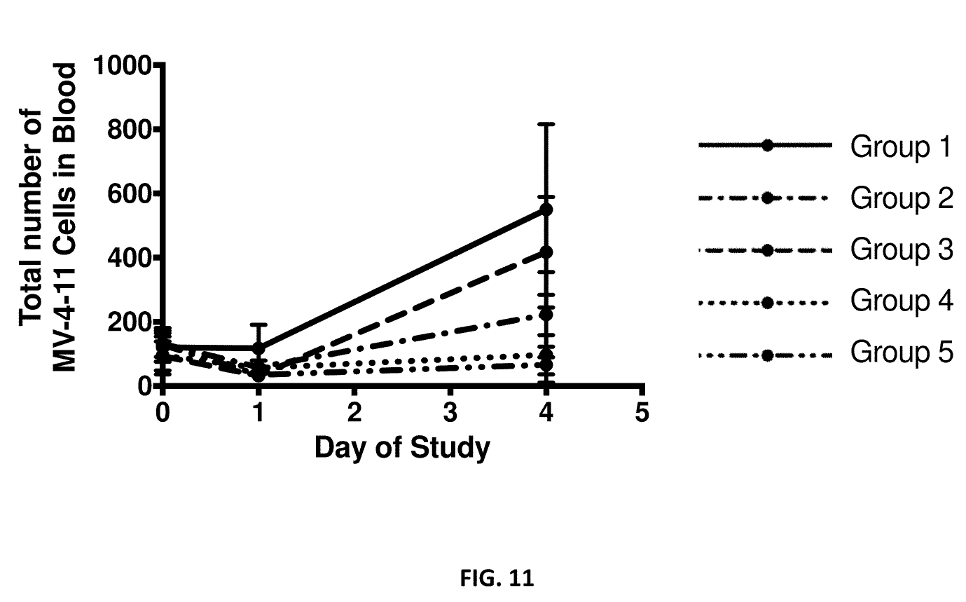

As shown in FIG. 11, treatment with either mRBC-PseuGLNase (Group 2) or mRBC-PseuGLNase/αCD33scFv (Groups 4 and 5) resulted in reduced tumor growth in the peripheral blood as compared to the control Group 1. Treatment with mRBC-PseuGLNase/αCD33scFv (Groups 4 and 5) resulted in greater suppression of tumor growth in the peripheral blood than treatment with mRBC-PseuGLNase (Group 2). This was particularly surprising since mRBC-PseuGLNase/αCD33scFv (Groups 4 and 5) have less enzymatic activity than mRBC-PseuGLNase (Group 2) (i.e., 0.054 (Group 4) and 0.026 (Group 5) vs. 0.092 (Group 2) as shown in Table 11).

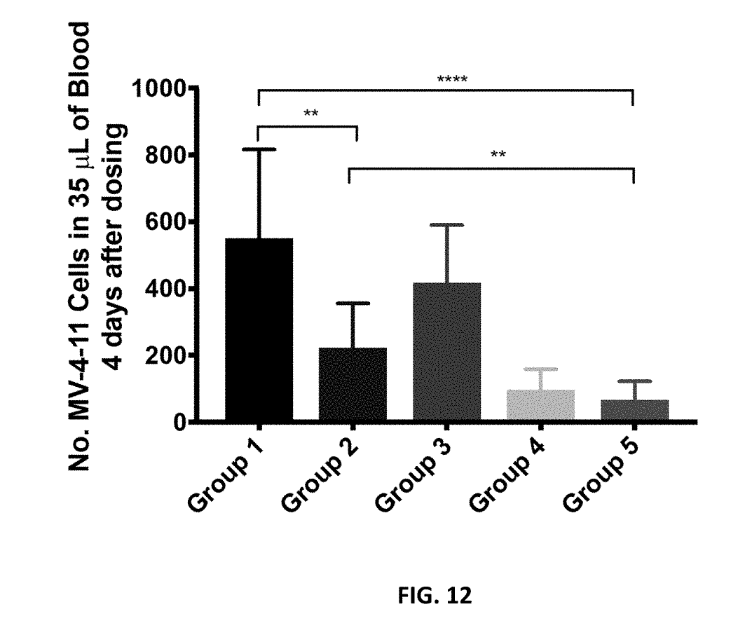

Moreover, FIG. 12 shows that treatment with mRBC-PseuGLNase (Group 2) and mRBC-PseuGLNase/αCD33scFv (Groups 4 and 5) resulted in a statistically significant suppression of tumor growth at day 4 post-administration as compared to the control Group 1. Further, treatment with mRBC-PseuGLNase/αCD33scFv (Groups 4 and 5) resulted in a statistically significant greater reduction in tumor load as compared to treatment with mRBC-PseuGLNase (Group 2).

Of interest, treatment with mRBC-EcoliASNase (Group 3) did not reduce MV4-11 tumor burden as well as mRBC-PseuGLNase (Group 2) (FIG. 11). Without wishing to be bound by any particular theory, the difference in outcome may be due to differences in the enzymatic activities of EcoliASNase and PseuGLNase as both glutamine and asparagine depletion may be needed for inducing death of MV4-11 cells, and EcoliASNase has limited glutaminase activity (see, e.g., Nguyen et al. (2018) Cancer Res. 78(6): 1549-60).

In sum, targeting of enucleated erythroid cells comprising an amino acid degradative enzyme to target cells (such as cancer cells) using a cell targeting moiety (e.g., an exogenous polypeptide comprising an anti-CD33 scFv) resulted in greater in vivo efficacy and tumor burden reduction despite lesser enzymatic activity.

### Example 9. Recombinant PseuGLNase Induces Cell Death in Multiple Cancer Cell Lines

To determine whether several known cancer cell lines were sensitive to treatment with the amino acid degradative enzyme PseuGLNase, which has dual asparaginase- and glutaminase-depleting activity, the following experiments was performed. The cancer cell lines MV4-11, MOLM-13, THP1, HL60, B16-F10, RPMI 8226, and CD34+ hematopoietic stem cells (control) were contacted with either 0.1 μg, 0.5 μg, or 1 μg of recombinant PseuGLNase in 1 mL of culture media containing each of the cell lines. Cells were harvested after a 68-87 hour incubation period with the recombinant PseuGLNase, and the number of remaining live cells post-treatment was determined. Culture media asparagine and glutamine concentration was analyzed using high performance liquid chromatography (HPLC) after the PseuGLNase incubation period to determine whether both amino acids were depleted. A summary of the experimental set-up, including the length of incubation with recombinant PseuGLNase, is provided in Table 12.

TABLE 12
Cells tested for sensitivity to recombinant PseuGLNase treatment.

| | | | | Hours |
|---|---|---|---|---|
| | | | Initial | contacted |
| | | ATCC | seeding | with |
| | | Deposit | density | recombinant |
| Cell Name | Cancer Type | Number | (cells/well) | PseuGLNase |
| MV4-11 | Biphenotypic B | CRL-9591 | ~4.9 × 10⁴ | 87 |
| | myelomonocytic | | | |
| | leukemia | | | |
| MOLM-13 | Acute myeloid | N/A | ~3.7 × 10⁴ | 87 |
| | leukemia | | | |
| THP1 | Actue monocytic | TIB-202 | ~3.9 × 10⁴ | 71 |
| | leukemia | | | |
| HL60 | Acute | CCL-240 | ~3.7 × 10⁴ | 71 |
| | promyelocytic | | | |
| | leukemia | | | |
| B16-F10 | Melanoma | CRL-6475 | ~1.7 × 10⁴ | 67 |
| RPMI 8226 | Plasmacytoma; | CCL-155 | ~2.6 × 10⁴ | 68 |
| | myeloma | | | |
| CD34 + | N/A | N/A | ~4.8 × 10⁴ | 71 |
| hematopoietic | | | | |
| stem cells | | | | |
| (control) | | | | |

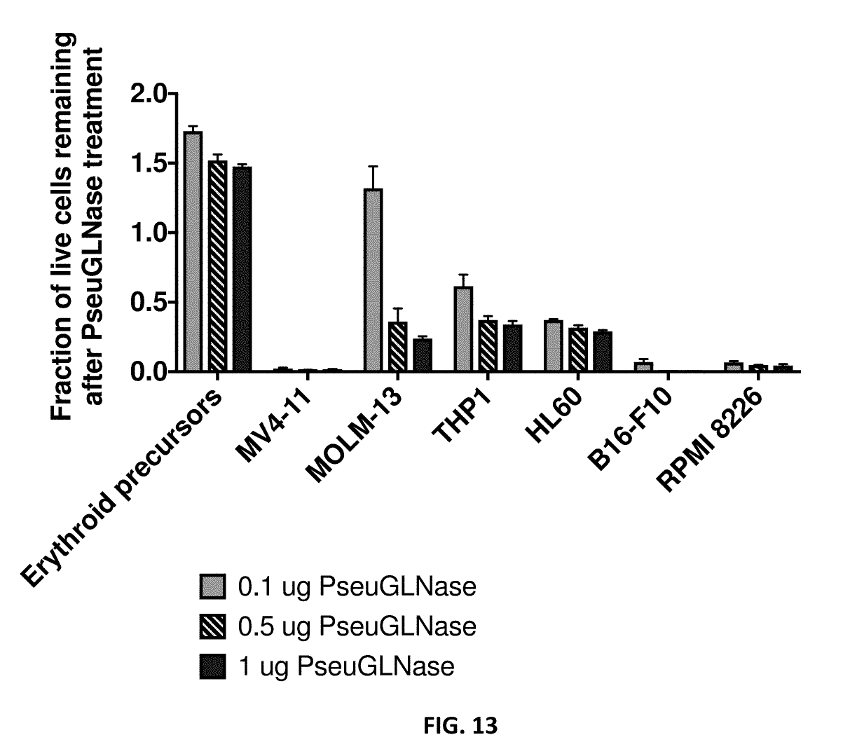

Treatment with either 0.1 μg, 0.5 μg, or 1 μg of recombinant PseuGLNase completely depleted the asparagine and glutamine present in the culture media after the incubation period (data not shown). As shown in FIG. 13, treatment with PseuGLNase does not induce cell death in CD34+ hematopoietic stem cells (“erythroid precursors”). In contrast, treatment with PseuGLNase induced cell death in all of the tested cancer cell lines (i.e., MV4-11, MOLM-13, THP1, HL60, B16-F10, and RPMI 8226). (Note: in FIG. 13, fraction of live cells remaining represents the number of live cells post-treatment divided by the number of live cells pre-treatment; the fraction of live cells below <1 indicates cell death, whereas fraction of live cells remaining >1 indicates cell growth). Thus, treatment with recombinant PseuGLNase induces cell death in a variety of cancer cell lines.

### Example 10. Generation of Genetically Engineered Enucleated Erythroid Cells Comprising PseuGLNase

Enucleated erythroid cells comprising a fusion protein comprising SMIM1 (transmembrane protein) fused to PseuGLNase via a linker containing HA epitope tag (referred to in this example as SMIM1-HA-PseuGLNase; see SEQ ID NO: 115) were generated. The HA epitope tag was included to allow for detection of the SMIM1-HA-PseuGLNase protein present on the surface of the cells using a PE-conjugated anti-HA tag antibody.

Production of Lentiviral Vector

The gene encoding SMIM1-HA-PseuGLNase was cloned into the multiple cloning site of the pLVX-TetOne lentivirus vector (Takara Bio), under the control of the P-TRE3GS promoter and the Tet-On 3G transactivator. Lentivirus was produced in Hyper-Flask seeded with HEK293T cells by transfecting the cells with viral packaging vectors and the pLVX-TetOne vector containing the gene encoding SMIM1-HA-PseuGLNase in OptiMEM. Lentiviral supernatant was collected 72 hours post-transfection and clarified by centrifugation at 1,500 rpm for 5 minutes. The clarified supernatant was then concentrated by TFF and the lentivirus particles pelleted by ultracentrifugation. The lentivirus pellets were then resuspended and pooled in a final volume of 3 mL IMDM and then frozen in aliquots at −80° C.

Concentrated viral titers were determined by transducing HEK-293T cells with serial dilutions of the concentrated lentivirus by spinoculation, spinning the plate at 2000 rpm for 90 minutes at room temperature. After spinoculation, doxycycline was added to each well at a final concentration of 3 μg/mL. Cells were maintained in an incubator at 37° C., 5% CO₂ for 72 hours before lentivirus titers were determined by staining for HA tag and assaying by flow cytometry.

Transduction, Expansion, and Differentiation of Erythroid Precursor Cells

Erythroid precursor cells were transduced during step 1 of the culture process described above. Erythroid cells in culturing medium were combined with concentrated lentivirus at a MOI of 40, and poloxamer 338 (1 mg/ml). The cells were incubated at 37° C. overnight. Human CD34+ cells were expanded and differentiated as described in Example 2.

Induction of SMIM1-HA-PseuGLNase Expression

Expression of SMIM1-HA-PseuGLNase was induced by adding doxycycline (3 μg/mL) on fifth day of the third stage of erythroid cell maturation. This concentration of doxycycline was maintained steady until cells were harvested on the ninth day of the third stage.

Detection of SMIM1-HA-PseuGLNase in Enucleated Erythroid Cells

To detect SMIM1-HA-PseuGLNase expression, the resulting enucleated erythroid cells were incubated with fluorophore-conjugated anti-HA tag antibody which binds to the HA tag present in the linker of the protein. The cells were analyzed by flow cytometry. A gate was set based on stained untransduced cells.

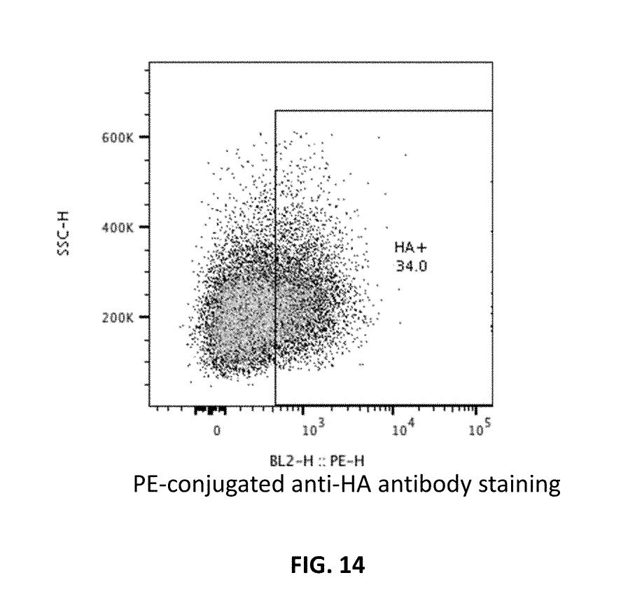

As shown in FIG. 14, approximately 34% of the enucleated erythroid cells had detectable SMIM1-HA-PseuGLNase protein on the cell surface.

1. A genetically engineered, enucleated erythroid cell comprising:
a first exogenous polypeptide comprising an amino acid degradative enzyme; and
a second exogenous polypeptide comprising a cell targeting moiety.

2. The genetically engineered, enucleated erythroid cell of claim 1, wherein the amino acid degradative enzyme is an asparaginase molecule.

3-6. (canceled)

7. The genetically engineered, enucleated erythroid cell of claim 1, wherein the amino acid degradative enzyme comprises an asparaginase molecule, wherein the asparaginase molecule comprises an amino acid sequence having at least 80% identity to an amino acid sequence of any of SEQ ID NOs: 3-8 or 68-70.

8-16. (canceled)

17. The genetically engineered, enucleated erythroid cell of claim 1, wherein the amino acid degradative enzyme:
(i) comprises a serine dehydratase molecule, a serine hydroxymethyltransferase molecule, an arginase-1 molecule, an arginine deiminase molecule, an L-methionine gamma-lyase molecule, an L-amino-acid oxidase molecule, an S-adenosylmethionine synthase molecule, a cystathionine gamma-lyase molecule, a NAD-dependent L-serine dehydrogenase molecule, an indoleamine 2,3-dioxygenase molecule, or a phenylalanine ammonia lyase molecule; and/or
(ii) comprises an amino acid sequence set forth in Table 3, or an amino acid sequence having at least 70% identity thereto.

18-20. (canceled)

21. The genetically engineered, enucleated erythroid cell of claim 1, wherein the first exogenous polypeptide further comprises a transmembrane domain, and wherein the transmembrane domain comprises:
(a) a transmembrane region of a type 1 membrane protein, a type 2 membrane protein, or a type 3 membrane protein;
(b) a transmembrane domain of a protein or a transmembrane polypeptide having at least 70%, 80%, 85%, 90%, 95%, 96%, 97%, 98%, or 99% identity to the amino acid sequence of any one of SEQ ID NOs: 20, 24, 26, or 27; or
(c) a transmembrane region of Kell.

22-31. (canceled)

32. The genetically engineered, enucleated erythroid cell of claim 1, wherein the amino acid degradative enzyme is present on the surface of the erythroid cell.

33. The genetically engineered, enucleated erythroid cell of claim 1, wherein the amino acid degradative enzyme is inside the erythroid cell.

34. (canceled)

35. The genetically engineered, enucleated erythroid cell of claim 1, wherein the cell targeting moiety comprises an antibody molecule, a ligand or a receptor, wherein the antibody molecule, ligand or receptor specifically bind to a cell surface marker.

36. The genetically engineered, enucleated erythroid cell of claim 35, wherein the cell targeting moiety is an antibody molecule, and wherein the antibody molecule comprises a single chain antibody, an (scFv)₂, a nanobody, or a camelid antibody.

37. The genetically engineered, enucleated erythroid cell of claim 1, wherein the cell targeting moiety specifically binds to a cell surface marker of a target cell.

38. The genetically engineered, enucleated erythroid cell of claim 37, wherein the target cell is a cancer cell.

39. (canceled)

40. The genetically engineered, enucleated erythroid cell of claim 38, wherein the cancer cell is selected from a leukemia cell, an acute myeloid leukaemia (AML) cell, an acute lymphoblastic leukaemia (ALL) cell, an anal cancer cell, a bile duct cancer cell, a bladder cancer cell, a bone cancer cell, a bowel cancer cell, a brain tumor cell, a breast cancer cell, a carcinoid cell, a cervical cancer cell, a choriocarcinoma cell, a chronic lymphocytic leukaemia (CLL) cell, a chronic myeloid leukaemia (CML) cell, a colon cancer cell, a colorectal cancer cell, an endometrial cancer cell, an eye cancer cell, a gallbladder cancer cell, a gastric cancer cell, a gestational trophoblastic tumor (GTT) cell, a hairy cell leukaemia cell, a head and neck cancer cell, a Hodgkin lymphoma cell, a kidney cancer cell, a laryngeal cancer cell, a liver cancer cell, a lung cancer cell, a lymphoma cell, a melanoma cell, a skin cancer cell, a mesothelioma cell, a mouth or oropharyngeal cancer cell, a myeloma cell, a nasal or sinus cancer cell, a nasopharyngeal cancer cell, a non-Hodgkin lymphoma (NHL) cell, an oesophageal cancer cell, an ovarian cancer cell, a pancreatic cancer cell, a penile cancer cell, a prostate cancer cell, a rectal cancer cell, a salivary gland cancer cell, a skin cancer cell, a soft tissue sarcoma cell, a stomach cancer cell, a testicular cancer cell, a thyroid cancer cell, a uterine cancer cell, a vaginal cancer cell, and a vulval cancer cell.

41. (canceled)

42. The genetically engineered, enucleated erythroid cell of claim 1, wherein the cell targeting moiety comprises:
(i) an antibody molecule comprising six CDRs from an antibody molecule of Table 7, wherein CDRs are determined according to Kabat, Chothia, or a combination thereof; or
(ii) an antibody molecule having a VH domain and a VL domain from Table 7, or an antigen-binding polypeptide having at least 70%, 80%, 85%, 90%, 95%, 96%, 97%, 98%, or 99% identity thereto.

43-45. (canceled)

46. The genetically engineered, enucleated erythroid cell of claim 1, wherein the cell targeting moiety specifically binds to CD33, CD20, CD4, BCMA, PSA, CD269, CD123, CD47, or CD28.

47-53. (canceled)

54. A pharmaceutical composition comprising a population of the genetically engineered, enucleated erythroid cells of claim 1 and a pharmaceutically acceptable excipient.

55. A device comprising:
a container, and
a plurality of genetically engineered, enucleated erythroid cells of claim 1 disposed in the container.

56. (canceled)

57. A method of treating a cancer, comprising administering to a subject in need thereof a therapeutically effective amount of the genetically engineered, enucleated erythroid cell of claim 1.

58. (canceled)

59. The method of claim 57, wherein the cancer is leukemia, acute lymphoblastic leukaemia (ALL), anal cancer, bile duct cancer, bladder cancer, bone cancer, bowel cancer, brain tumors, breast cancer, cancer of unknown primary, cancer spread to bone, cancer spread to brain, cancer spread to liver, cancer spread to lung, carcinoid, cervical cancer, choriocarcinoma, chronic lymphocytic leukaemia (CLL), chronic myeloid leukaemia (CML), colon cancer, colorectal cancer, endometrial cancer, eye cancer, gallbladder cancer, gastric cancer, gestational trophoblastic tumors (GTT), hairy cell leukaemia, head and neck cancer, Hodgkin lymphoma, kidney cancer, laryngeal cancer, leukaemia, liver cancer, lung cancer, lymphoma, melanoma skin cancer, mesothelioma, men's cancer, molar pregnancy, mouth and oropharyngeal cancer, myeloma, nasal and sinus cancers, nasopharyngeal cancer, non-Hodgkin lymphoma (NHL), oesophageal cancer, ovarian cancer, pancreatic cancer, penile cancer, prostate cancer, rare cancers, rectal cancer, salivary gland cancer, secondary cancers, skin cancer, soft tissue sarcoma, stomach cancer, testicular cancer, thyroid cancer, unknown primary cancer, uterine cancer, vaginal cancer, or vulval cancer.

60-65. (canceled)

66. A method of targeting an amino acid degradative enzyme to a target cell in a subject or of reducing the concentration of an amino acid in a subject, comprising administering a composition of genetically engineered, enucleated erythroid cells of claim 1 to the subject.

67. (canceled)

68. A method of selecting a genetically engineered, enucleated erythroid cell for administration to a subject having a cancer, comprising:
(i)
(a) acquiring information about the sensitivity of a cancer to an amino acid degradative enzyme;
(b) responsive to (a), selecting a genetically engineered, enucleated erythroid cell comprising an amino acid degradative enzyme to which the cancer is sensitive; and
(c) administering the genetically engineered, enucleated erythroid cell comprising the amino acid degradative enzyme to the subject;
(ii)
(a) acquiring information about a cell surface marker of the cancer;
(b) responsive to (a), selecting a genetically engineered, enucleated erythroid cell comprising a cell targeting moiety that binds the cell surface marker; and
(c) administering the genetically engineered, enucleated erythroid cell comprising the cell targeting moiety to the subject; or
(iii)
(a) acquiring information about the sensitivity of a cancer to an amino acid degradative enzyme;
(b) acquiring information about a cell surface marker of the cancer;
(c) responsive to (a), selecting a genetically engineered, enucleated erythroid cell comprising an amino acid degradative enzyme to which the cancer is sensitive and further comprising a cell targeting moiety that binds the cell surface marker; and
(d) administering the genetically engineered, enucleated erythroid cell comprising the amino acid degradative enzyme and the cell targeting moiety to the subject.

69-70. (canceled)
